(19) United States
(12) Patent Application Publication
(10) Pub. No.: US 2026/0137736 A1
(43) Pub. Date: May 21, 2026

(54) PROBIOTIC LACTOBACILLUS STRAIN COMPOSITIONS AND APPLICATIONS THEREFOR INCLUDING FOR LEAKY GUT AND INFLAMMATION

(71) Applicant: BiomEdit, LLC, Fishers, IN (US)

(72) Inventors: Arvind Kumar, Fishers, IN (US); Dharanesh Mahimapura Gangaiah, Fishers, IN (US)

(21) Appl. No.: 18/704,175

(22) PCT Filed: Oct. 26, 2022
(86) PCT No.: PCT/US2022/047847

§ 371 Date: Apr. 24, 2024

Related U.S. Application Data
(60) Provisional application No. 63/272,100, filed on Oct. 26, 2021.

(51) Int. Cl. A61K 35/747; A61K 31/56; A61K 31/702; A61K 38/19; A61P 1/00; C12N 15/52

(52) U.S. Cl. CPC A61K 35/747; A61K 31/56; A61K 31/702; A61K 38/19; A61P 1/00; C12N 15/52; C12N 2800/101; C12N 2840/002

(57) ABSTRACT

The present invention provides probiotic compositions and methods for improving animal health. The probiotic compositions include one or more isolated strains of Lactobacillus reuteri which colonizes the gastrointestinal tract to increase the health of an animal, including to alleviate the effects of chronic alcohol consumption, leaky gut, increased intestinal permeability and inflammation and treat or prevent alcohol-associated intestinal dysbiosis, leaky gut, increased intestinal permeability and inflammation, including intestinal inflammation associated with inflammatory bowel disease.

### CROSS REFERENCE TO RELATED APPLICATIONS

The present application claims priority to U.S. Application Ser. No. 63/272,100, filed Oct. 26, 2021, the entire contents of which is incorporated by reference herein.

### SEQUENCE LISTING

This application contains a Sequence Listing, which was submitted in XML format via EFS-Web, and is hereby incorporated by reference in its entirety. The XML copy, created on Oct. 24, 2022, is named “2950-30PCT_ST26.xml” and is 5,069,323 bytes in size.

### FIELD OF THE INVENTION

The present invention relates to probiotic compositions and methods for improving animal health. The probiotic compositions include one or more isolated strains of Lactobacillus reuteri which colonizes the gastrointestinal tract to increase the health of an animal, including to alleviate the effects of chronic alcohol consumption, leaky gut, increased intestinal permeability and inflammation and treat or prevent alcohol-associated intestinal dysbiosis, leaky gut, increased intestinal permeability and inflammation.

### BACKGROUND

Direct fed microbials (DFMs), often also called probiotics, are microorganisms which colonize, at least temporarily, the gastrointestinal tract of an animal and provide some beneficial effect to that animal. The microorganisms can be bacterial species, for example those from the genera Bacillus, Lactobacillus, Lactococcus, and Enterococcus. The microorganisms can also be yeast or even molds. The microorganisms can be provided to an animal orally or mucosally or, in the case of birds, provided to a fertilized egg, i.e. in ovo.

The beneficial activity provided by a DFM or probiotics can be the synthesis of vitamins or other nutritional molecules needed for a healthy metabolism of the host animal. A DFM or probiotic can also protect the host animal from disease, disorders, or clinical symptoms caused by other, pathogenic microorganisms. For example, the DFM or probiotic may produce factors having inhibitory or cytotoxic activity against certain species of pathogens, such as deleterious or disease-causing bacteria, or immunomodulatory activity, such as modulating immune response or improving immune response to foreign agent(s) or foreign antigen. The DFM or probiotic may produce factors or biomolecules having therapeutic activity, including blocking or reducing inflammation or inflammatory response(s).

Alcohol use disorder (AUD) affects 15.1 million US adults and is associated with more severe medical illness (Fernandezsola J et al. (1995) Archives of Internal Medicine 155: 1649-1654; de Roux, A et al. (2006) Chest, 129:1219-1225). A high prevalence of pneumonia and other infections, with subsequent poor clinical outcomes, is found in alcohol drinkers, as both innate and adaptive immune cells are compromised by alcohol use (Gonzalez-Quintela A et al. (2008) Clinical and Experimental Immunology, 151:42-50; Greenberg, S S et al. (1999) Alcoholism, Clinical and Experimental Research, 23: 735-744; Mandrekar, P et al. (2004) The Journal of Immunology, 173: 3398-3407; Mason C M et al. (2004)) Infection and Immunity, 72:2556-2563; Samuelson, D R et al. (2017) PLoS Path, 13(6), e1006426). Alcohol consumption perturbs the normal intestinal microbial communities and induces gut dysbiosis. Binge-on-chronic alcohol feeding in animal models shifts gut microbiota diversity and functional capacity. Human and rodent studies have reported that excessive alcohol consumption changes gut microbiota composition, structure, and metabolic function (Leclercq S et al. (2014) Proceedings of the National Academy of Sciences of the United States of America, 111, E4485-E4493, 2014a; Mutlu, E A et al. (2012) American Journal of Physiology-Gastrointestinal and Liver Physiology, 302, G966-G978).

Recent studies have shown that alterations in the intestinal immune response as a consequence of alcohol-induced dysbiosis contribute to increased host susceptibility to Klebsiella pneumonia (Samuelson D. R. et al (2017) PLOS Pathogens 13(6): e1006426; doi.org/10.1371/journal.ppat.1006426). Alcohol-associated susceptibility to K. pneumoniae is, in part, mediated by gut dysbiosis, as alcohol-naïve animals recolonized with a microbiota isolated from alcohol-fed mice had an increased respiratory burden of K. pneumoniae compared to mice recolonized with a control microbiota. The increased susceptibility in alcohol dysbiosis recolonized animals was associated with an increase in pulmonary inflammatory

cytokines, and a decrease in the number of CD4+ and CD8+ T-cells in the lung following Klebsiella infection but an increase in T-cell counts in the intestinal tract following Klebsiella infection, suggesting intestinal T-cell sequestration as a factor in impaired lung host defense. Mice recolonized with an alcohol-dysbiotic microbiota also had increased intestinal damage as measured by increased levels of serum intestinal fatty acid binding protein.

Alcohol consumption also increases susceptibility to pneumococcal pneumonia, mediated by intestinal dysbiosis, including as demonstrated in a humanized murine HIV model (Samuelson D. R, et al (2019) Alcohol 80:33-43; doi.org/10.1016/j.alcohol.2018.08.012). The intestinal microbiota from alcohol-fed mice (regardless of HIV status) significantly impaired clearance of S. pneumoniae. The data indicate that alcohol feeding, as well as alcohol-associated intestinal dysbiosis, compromise pulmonary host defenses against infection, including infectious bacteria, such as pneumococcal pneumonia. The microbial community structure is altered and dysfunctional with significant alcohol consumption.

Intestines or guts are semi-permeable. The mucous lining of intestines is designed to absorb water and nutrients from food into the bloodstream. However, some animals or individuals have increased intestinal permeability or hyperpermeability, wherein their guts or intestines release more than water and nutrients through and can even permit other molecules or components to ‘leak’ through the intestines or gut. This can often be termed ‘leaky gut’. Leaky gut mechanism and clinical implications are reviewed in Camilleri, M (Camilleri M (2019) Gut 68(8):1516-1527; doi:10.1136/gutjnl-2019-318427).

As noted above, alcohol use can lead to increased intestinal permeability, intestinal dysbiosis, and leaky gut, in addition, studies have shown that people who have certain chronic gastrointestinal diseases have leaky guts that let larger molecules through, including potentially harmful or toxic ones. In as much as the intestinal lining importantly acts as a barrier to bacteria and other infectious agents inside the gut, this barrier is an important agent and component in the immune system. If the intestinal barrier is impaired, it can permit toxins and other harmful or inflammatory molecules to enter the bloodstream. These toxins may trigger an inflammatory response that may manifest as various diseases.

The most direct causes of altered or increased intestinal permeability include: chronic inflammatory states, such as IBD and celiac disease; other diseases that cause intestinal injury, such as HIV/AIDS; chemotherapy and radiation therapies that degrade the intestinal mucosa; chronic overuse of alcohol or NSAIDs, such as aspirin and ibuprofen; food allergies that cause an immune response to certain foods. Intestinal permeability is a recognized feature of several inflammatory and autoimmune diseases affecting the digestive system, including inflammatory bowel disease, Crohn's disease and celiac disease (Bjarnason I et al (1983) Lancet 1:323-5; Hollander D et al (1986) Ann Intern Med 1986; 105:883-5; Vazquez-Roque M I et al (2013) Gastroenterology 144:903-11, e3; D'Incà R et al (1999) Am J Gastroenterol 94:2956-60). These diseases cause chronic inflammation in the intestines, which leads to erosion of the intestinal barrier gradually over time. Higher levels of gut bacteria products have been measured in the blood in individuals with gastrointestinal (01) diseases that are known to cause intestinal permeability

There is a need in the art for approaches and therapies to mediate and alleviate the effects of chronic alcohol consumption, including intestinal dysbiosis, leaky gut, increased intestinal permeability and inflammation. There is a need for probiotic compositions to correct or address the altered and compromised intestinal microbial community with chronic alcohol consumption and intestinal diseases and inflammation and for methods that provide beneficial bacteria and/or molecules to the gastrointestinal tract of an animal and improve animal health, particularly in animals consuming alcohol on a chronic or binge basis, animals with leaky gut, increased intestinal permeability and intestinal-derived or intestinal-associated inflammation.

### SUMMARY OF THE INVENTION

The present invention provides compositions and methods for improving animal health. The probiotic compositions and methods improve the health of an animal, including in alleviating the effects of chronic alcohol consumption, leaky gut, increased intestinal permeability and inflammation and treat or prevent alcohol-associated intestinal dysbiosis, leaky gut, increased intestinal permeability and inflammation.

In one embodiment, the invention provides a composition having at least one isolated Lactobacillus reuteri strain, wherein said composition increases animal health when an effective amount is administered to an animal, as compared to an animal not administered the composition. In one embodiment, the invention provides a composition having at least one of an isolated first Lactobacillus reuteri strain and an isolated second Lactobacillus reuteri strain, wherein said composition increases animal health when an effective amount is administered to an animal, as compared to an animal not administered the composition.

In one embodiment, the invention provides a composition having at least one isolated Lactobacillus reuteri strain, wherein said composition increases animal health, including in alleviating the effects of chronic alcohol consumption, leaky gut, increased intestinal permeability and inflammation, when an effective amount is administered to an animal, as compared to an animal not administered the composition. In one embodiment, the invention provides a composition having at least one of an isolated first Lactobacillus reuteri strain and an isolated second Lactobacillus reuteri strain, wherein said composition increases animal health, including in alleviating the effects of chronic alcohol consumption, leaky gut, increased intestinal permeability and inflammation, when an effective amount is administered to an animal, as compared to an animal not administered the composition.

The invention provides methods for reducing intestinal permeability, alleviating alcohol induced or disease related leaky gut syndrome, alleviating the intestinal and systemic effects of chronic alcohol consumption, including intestinal dysbiosis, and reducing inflammation, including intestinal-derived or intestinal-associated inflammation, all and any of which include administration of an effective amount of an immunogenic probiotic composition comprising at least one Lactobacillus reuteri strain. In embodiments of the methods, a combination of two or at least two Lactobacillus reuteri strains are administered.

In one embodiment, the present disclosure provides a method of alleviating or modulating increased intestinal permeability or leaky gut in a subject. In one embodiment, the present disclosure provides a method of reducing intestinal permeability in a subject. The method includes administering an effective amount of a probiotic composition comprising at least one Lactobacillus reuteri strain. In an embodiment, the method includes administering an effective amount of an immunogenic probiotic composition comprising at least one Lactobacillus reuteri strain wherein the at least one Lactobacillus reuteri strain comprises at least one sequence selected from SEQ ID NO:1-55 and sequences having at least 98%, at least 98.5%, at least 99%, or at least 99.5% sequence identity with at least one of SEQ ID NO:1-55. In an embodiment, the method comprises administering an effective amount of a probiotic composition comprising at least one Lactobacillus reuteri strain, and optionally, an anti-inflammatory agent, molecule or cytokine, to a subject.

In one embodiment, the present disclosure provides a method of reducing inflammation, including intestinal-derived or intestinal-associated inflammation, including inflammation associated with intestinal disease or with increased intestinal permeability, in a subject. The method includes administering an effective amount of an immunogenic probiotic composition comprising at least one Lactobacillus reuteri strain and wherein the at least one Lactobacillus reuteri strain comprises at least one sequence selected from SEQ ID NO: 1-55 and sequences having at least 98%, at least 98.5%, at least 99%, or at least 99.5% sequence identity with at least one of SEQ ID NO:1-55. In an embodiment, the method comprises administering an effective amount of a probiotic composition comprising at least one Lactobacillus reuteri strain, and optionally, an anti-inflammatory agent, molecule or cytokine, to a subject. In an embodiment, the method comprises administering an effective amount of a probiotic composition comprising at least one Lactobacillus reuteri strain, and optionally, an immunomodulatory agent or molecule, to a subject.

In an embodiment of the method(s), a combination of two isolated Lactobacillus reuteri strains are administered. In embodiments, an anti-inflammatory agent, molecule or cytokine or an immunomodulatory agent is administered in combination with the at least one Lactobacillus reuteri strain or with the two isolated Lactobacillus reuteri strains. The at least one or the two Lactobacillus reuteri strains may be administered prior to one or more anti-inflammatory agent, molecule or cytokine or immunomodulator; may be administered prior to and in conjunction one or more anti-inflammatory agent, molecule or cytokine or immunomodulator; may be administered prior to, in conjunction with, and following one or more anti-inflammatory agent, molecule or cytokine or immunomodulator; or may be administered in combination with or shortly following one or more anti-inflammatory agent, molecule or cytokine or immunomodulator. The one or more anti-inflammatory agent, molecule or cytokine or immunomodulator may be administered as a single dose or multiple doses. The at least one or the two Lactobacillus reuteri strains may be administered prior to and/or between and/or in combination with a dose of one or more anti-inflammatory agent, molecule or cytokine or immunomodulator or multiple doses of one or more anti-inflammatory agent, molecule or cytokine or immunomodulator.

In an embodiment of the method(s) of the invention, a combination of two isolated Lactobacillus reuteri strains includes or comprises or is a combination of a first isolated Lactobacillus reuteri strain and a second isolated Lactobacillus reuteri strain. In one embodiment, the isolated first Lactobacillus reuteri strain includes at least one of: a nucleic acid sequence having at least 95%, at least 96%, at least 97%, at least 98%, or at least 99% sequence identity with SEQ ID NO:26, a nucleic acid sequence having at least 95%, at least 96%, at least 97%, at least 98%, or at least 99% sequence identity with SEQ ID NO: 1, a nucleic acid sequence having at least 95%, at least 96%, at least 97%, at least 98%, or at least 99% sequence identity with SEQ ID NO: 3, and a nucleic acid that encodes for an amino acid sequence having at least 95%, at least 96%, at least 97%, at least 98%, or at least 99% sequence identity with SEQ ID NO: 8.

In one embodiment, the second isolated second Lactobacillus reuteri strain includes at least one of: a nucleic acid sequence having at least 95%, at least 96%, at least 97%, at least 98%, or at least 99% sequence identity with SEQ ID NO:25, a nucleic acid sequence having at least 95%, at least 96%, at least 97%, at least 98%, or at least 99% sequence identity with SEQ ID NO: 27, a nucleic acid sequence having at least 95%, at least 96%, at least 97%, at least 98%, or at least 99% sequence identity with SEQ ID NO: 28, and a nucleic acid sequence having at least 95%, at least 96%, at least 97%, at least 98%, or at least 99% sequence identity with SEQ ID NO: 29.

In one embodiment, the isolated first Lactobacillus reuteri strain has a nucleic acid or amino acid sequence including at least one of SEQ ID NOs: 1-24, 26, and 49-55, sequences having at least one nucleic acid or amino acid sequence difference from the sequence of at least one of SEQ ID NOs: 1-24, 26, and 49-55, sequences having one or more nucleic acid or amino acid sequence difference from the sequence of at least one of SEQ ID NOs: 1-24, 26, and 49-55, sequences having at least one nucleic acid or amino acid sequence difference from the sequence of at least one of SEQ ID NOs: 1-24, 26, and 49-55 and further having at least 99% sequence identity with at least one of SEQ ID NOs: 1-24, 26, and 49-55, sequences having at least one nucleic acid or amino acid sequence difference from the sequence of at least one of SEQ ID NOs: 1-24, 26, and 49-55 and further having at least 99% sequence identity with one or more of SEQ ID NOs: 1-24, 26, and 49-55, sequences having at least 98%, at least 98.5%, at least 99%, or at least 99.5% sequence identity with at least one of SEQ ID NOs: 1-24, 26, and 49-55. In one embodiment, the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55.

In an embodiment, the isolated first Lactobacillus reuteri strain is Lactobacillus reuteri strain 3632, which corresponds to ATCC Patent Deposit Number PTA-126788.

In one embodiment, the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788.

In some embodiments, the isolated second Lactobacillus reuteri strain has a nucleic acid sequence or amino acid sequence including at least one of SEQ ID NOs: 25, 27-43, and 44-48, sequences having at least 98%, at least 98.5%, at least 99%, or at least 99.5% sequence identity with at least one of SEQ ID NOs: 25, 27-43, and 44-48.

In one embodiment, the isolated second Lactobacillus reuteri has a nucleic acid or amino acid sequence including at least one of SEQ ID NOs: 25, 27-43, and 44-48, sequences having at least one nucleic acid or amino acid sequence difference from the sequence of at least one of SEQ ID NOs: 25, 27-43, and 44-48, sequences having one or more nucleic acid or amino acid sequence difference from the sequence of at least one of SEQ ID NOs: 25, 27-43, and 44-48, sequences having at least one nucleic acid or amino acid sequence difference from the sequence of at least one of SEQ ID NOs: 25, 27-43, and 44-48 and further having at least 99% sequence identity with at least one of SEQ ID NOs: 25, 27-43, and 44-48, sequences having at least one nucleic acid or amino acid sequence difference from the sequence of at least one of SEQ ID NOs: 25, 27-43, and 44-48 and further having at least 99% sequence identity with one or more of SEQ ID NOs: 25, 27-43, and 44-48, sequences having at least 98%, at least 98.5%, at least 99%, or at least 99.5% sequence identity with at least one of SEQ ID NOs: 25, 27-43, and 44-48. In one embodiment, the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

In an embodiment, the second Lactobacillus reuteri is Lactobacillus reuteri strain 3630, which corresponds to ATCC Patent Deposit Number PTA-126787.

In one embodiment, the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

In one embodiment, the present disclosure provides a method of reducing intestinal permeability or alleviating leaky gut in a subject. The method includes administering an effective amount of a probiotic composition comprising at least one Lactobacillus reuteri strain and wherein the at least one Lactobacillus reuteri strain comprises at least one sequence selected from SEQ ID NO: 1-55 and sequences having at least 98%, at least 98.5%, at least 99%, or at least 99.5% sequence identity with at least one of SEQ ID NO:1-55 to a subject. The method includes administering an effective amount of a probiotic composition comprising two isolated Lactobacillus reuteri strains, a first Lactobacillus reuteri strain and a second Lactobacillus reuteri strain, wherein the first Lactobacillus reuteri strain comprises at least one sequence selected from SEQ ID NO: 1-24, 26, and 49-55 and sequences having at least 98%, at least 98.5%, at least 99%, or at least 99.5% sequence identity with at least one of SEQ ID NO: 1-24, 26, and 49-55, wherein the second Lactobacillus reuteri strain comprises at least one sequence selected from SEQ ID NO: 25, 27-43, and 44-48 and sequences having at least 98%, at least 98.5%, at least 99%, or at least 99.5% sequence identity with at least one of SEQ ID NO: 25, 27-43, and 44-48 to a subject.

In one embodiment, the present disclosure provides a method of reducing inflammation, including intestinal-derived or intestinal-associated inflammation, including inflammation associated with intestinal disease or with increased intestinal permeability, in a subject. The method includes administering an effective amount of a probiotic composition comprising at least one Lactobacillus reuteri strain and wherein the at least one Lactobacillus reuteri strain comprises at least one sequence selected from SEQ ID NO:1-55 and sequences having at least 98%, at least 98.5%, at least 99%, or at least 99.5% sequence identity with at least one of SEQ ID NO: 1-55 to a subject. The method includes administering an effective amount of a probiotic composition comprising two isolated Lactobacillus reuteri strains, a first Lactobacillus reuteri strain and a second Lactobacillus reuteri strain, wherein the first Lactobacillus reuteri strain comprises at least one sequence selected from SEQ ID NO: 1-24, 26, and 49-55 and sequences having at least 98%, at least 98.5%, at least 99%, or at least 99.5% sequence identity with at least one of SEQ ID NO: 1-24, 26, and 49-55, wherein the second Lactobacillus reuteri strain comprises at least one sequence selected from SEQ ID NO: 25, 27-43, and 44-48 and sequences having at least 98%, at least 98.5%, at least 99%, or at least 99.5% sequence identity with at least one of SEQ ID NO: 25, 27-43, and 44-48 to a subject. The method includes further administering one or more anti-inflammatory agent, molecule or cytokine or immunomodulatory to a subject. The method includes further administering, including in combination in the composition, one or more anti-inflammatory agent, molecule or cytokine or immunomodulatory to a subject.

In one embodiment, the present disclosure provides a method of reducing intestinal inflammation associated with gastrointestinal disease including or such as inflammatory bowel disease (IBD), Crohn's disease, or celiac disease in a subject. The method includes administering an effective amount of a probiotic composition comprising at least one Lactobacillus reuteri strain and wherein the at least one Lactobacillus reuteri strain comprises at least one sequence selected from SEQ ID NO:1-55 and sequences having at least 98%, at least 98.5%, at least 99%, or at least 99.5% sequence identity with at least one of SEQ ID NO: 1-55 to a subject. The method includes administering an effective amount of a probiotic composition comprising two isolated Lactobacillus reuteri strains, a first Lactobacillus reuteri strain and a second Lactobacillus reuteri strain, wherein the first Lactobacillus reuteri strain comprises at least one sequence selected from SEQ ID NO: 1-24, 26, and 49-55 and sequences having at least 98%, at least 98.5%, at least 99%, or at least 99.5% sequence identity with at least one of SEQ ID NO: 1-24, 26, and 49-55, wherein the second Lactobacillus reuteri strain comprises at least one sequence selected from SEQ ID NO: 25, 27-43, and 44-48 and sequences having at least 98%, at least 98.5%, at least 99%, or at least 99.5% sequence identity with at least one of SEQ ID NO: 25, 27-43, and 44-48 to a subject. The method includes further administering one or more anti-inflammatory agent, molecule or cytokine or immunomodulatory to a subject. The method includes further administering, including in combination in the composition, one or more anti-inflammatory agent, molecule or cytokine or immunomodulatory to a subject.

In one embodiment, the present disclosure provides a method of reducing inflammation in the gut or intestine in a subject. The method includes administering an effective amount of a probiotic composition comprising at least one Lactobacillus reuteri strain and wherein the at least one Lactobacillus reuteri strain comprises at least one sequence selected from SEQ ID NO: 1-55 and sequences having at least 98%, at least 98.5%, at least 99%, or at least 99.5% sequence identity with at least one of SEQ ID NO: 1-55 to a subject. The method includes administering an effective amount of a probiotic composition comprising two isolated Lactobacillus reuteri strains, a first Lactobacillus reuteri strain and a second Lactobacillus reuteri strain, wherein the first Lactobacillus reuteri strain comprises at least one sequence selected from SEQ ID NO: 1-24, 26, and 49-55 and sequences having at least 98%, at least 98.5%, at least 99%, or at least 99.5% sequence identity with at least one of SEQ ID NO: 1-24, 26, and 49-55, wherein the second Lactobacillus reuteri strain comprises at least one sequence selected from SEQ ID NO: 25, 27-43, and 44-48 and sequences having at least 98%, at least 98.5%, at least 99%, or at least 99.5% sequence identity with at least one of SEQ ID NO: 25, 27-43, and 44-48 to a subject. The method includes further administering one or more anti-inflammatory agent, molecule or cytokine or immunomodulatory to a subject. The method includes further administering, including in combination in the composition, one or more anti-inflammatory agent, molecule or cytokine or immunomodulatory to a subject.

In one embodiment, the present disclosure provides a method of alleviating the intestinal effects of chronic alcohol consumption, including intestinal dysbiosis, in a subject. The method includes administering an effective amount of a probiotic composition comprising at least one Lactobacillus reuteri strain and wherein the at least one Lactobacillus reuteri strain comprises at least one sequence selected from SEQ ID NO: 1-55 and sequences having at least 98%, at least 98.5%, at least 99%, or at least 99.5% sequence identity thereto to a subject. The method includes administering an effective amount of a probiotic composition comprising two isolated Lactobacillus reuteri strains, a first Lactobacillus reuteri strain and a second Lactobacillus reuteri strain, wherein the first Lactobacillus reuteri strain comprises at least one sequence selected from SEQ ID NO: 1-24, 26, and 49-55 and sequences having at least 98%, at least 98.5%, at least 99%, or at least 99.5% sequence identity with at least one of SEQ ID NO: 1-24, 26, and 49-55, wherein the second Lactobacillus reuteri strain comprises at least one sequence selected from SEQ ID NO: 25, 27-43, and 44-48 and sequences having at least 98%, at least 98.5%, at least 99%, or at least 99.5% sequence identity with at least one of SEQ ID NO: 25, 27-43, and 44-48 to a subject.

In one embodiment, the present disclosure provides a probiotic composition. The composition includes at least one Lactobacillus reuteri strain and wherein the at least one Lactobacillus reuteri strain comprises at least one sequence selected from SEQ ID NO: 1-55 and sequences having at least 98%, at least 98.5%, at least 99%, or at least 99.5% sequence identity with at least one of SEQ ID NO: 1-55, and a pharmaceutically acceptable carrier. The composition includes at least one Lactobacillus reuteri strain and wherein the at least one Lactobacillus reuteri strain comprises at least one sequence selected from SEQ ID NO:44-48 or SEQ ID NO:49-55 and sequences having at least 98%, at least 98.5%, at least 99%, or at least 99.5% sequence identity with at least one of SEQ ID NO:44-48 or SEQ ID NO:49-55, and a pharmaceutically acceptable carrier. In an embodiment, the immunogenic probiotic composition comprises two isolated Lactobacillus reuteri strains, a first Lactobacillus reuteri strain and a second Lactobacillus reuteri strain, wherein the first Lactobacillus reuteri strain comprises at least one sequence selected from SEQ ID NO: 1-24, 26, and 49-55 and sequences having at least 98%, at least 98.5%, at least 99%, or at least 99.5% sequence identity with at least one of SEQ ID NO: 1-24, 26, and 49-55, wherein the second Lactobacillus reuteri strain comprises at least one sequence selected from SEQ ID NO: 25, 27-43, and 44-48 and sequences having at least 98%, at least 98.5%, at least 99%, or at least 99.5% sequence identity with at least one of SEQ ID NO: 25, 27-43, and 44-48.

In one embodiment, the first isolated first Lactobacillus reuteri strain includes at least one of: a nucleic acid sequence having at least 97%, at least 98%, or at least 99% sequence identity with SEQ ID NO:26, a nucleic acid sequence having at least 97%, at least 98%, or at least 99% sequence identity with SEQ ID NO: 1, a nucleic acid sequence having at least 95%, at least 96%, at least 97%, at least 98%, or at least 99% sequence identity with SEQ ID NO: 3, and a nucleic acid that encodes for an amino acid sequence having at least 97%, at least 98%, or at least 99% sequence identity with SEQ ID NO: 8.

In one embodiment, the second isolated second Lactobacillus reuteri strain includes at least one of: a nucleic acid sequence having at least 97%, at least 98%, or at least 99% sequence identity with SEQ ID NO:25, a nucleic acid sequence having at least 97%, at least 98%, or at least 99% sequence identity with SEQ ID NO: 27, a nucleic acid sequence having at least 97%, at least 98%, or at least 99% sequence identity with SEQ ID NO: 28, and a nucleic acid sequence having at least 97%, at least 98%, or at least 99% sequence identity with SEQ ID NO: 29.

In an embodiment, biosynthetic gene cluster (BGC), particularly a polyketide synthase (PKS) BGC is provided. In an embodiment, biosynthetic gene cluster (BGC), particularly a polyketide synthase (PKS) BGC of Lactobacillus reuteri strain 3632 is provided. In one embodiment, the PKS BGC is capable of producing an AhR-activating metabolite.

In some embodiments, the PKS BGC comprises the nucleic acid set out in SEQ ID NO: 77 is provided. In an embodiment, the PKS BGC comprises nucleic acid encoding one or more polypeptide selected from SEQ ID NOs: 78-92. In an embodiment, the PKS BGC comprises nucleic acid encoding one or more polypeptide selected from SEQ ID NOs: 78-85 and 87-92. In an embodiment, the PKS BGC comprises nucleic acid encoding the polypeptides SEQ ID NOs: 78-85 and 87-92. In an embodiment, an Ahr-metabolite synthesizing cluster of genes encoding proteins SEQ ID NO: 78-92, or at least SEQ ID NO: 78-85 and SEQ ID NO: 86-92 is provided.

In other embodiments, a plasmid is provided herein comprising nucleic acid encoding one or more polypeptide selected from SEQ ID NOs: 78-92. In an embodiment, the plasmid comprises nucleic acid encoding one or more polypeptide selected from SEQ ID NOs: 78-85 and 87-92. In an embodiment, the plasmid comprises nucleic acid encoding the polypeptides SEQ ID NOs: 78-85 and 87-92. In an embodiment, a plasmid comprising an Ahr-metabolite synthesizing cluster of genes encoding proteins SEQ ID NO: 78-92, or at least SEQ ID NO: 78-85 and SEQ ID NO: 86-92 is provided.

In embodiments, a gene cluster encoding an Ahr-metabolite, or a plasmid comprising a gene cluster encoding an Ahr-metabolite are provided. The Ahr-metabolite is relevant for IL-22 production. The Ahr-metabolite is capable of increasing IL-22 levels in the intestine. The Ahr-metabolite is capable of ameliorating or reducing intestinal inflammation. The Ahr-metabolite is relevant for and capable of maintaining intestinal barrier integrity. The Ahr-metabolite is relevant for and capable of reducing or alleviating increased intestinal permeability. In embodiments, increased expression or production of the PKS gene cluster, or increasing proteins SEQ ID NO:78-92, or SEQ ID NO:78-85 and 87-92 is contemplated herein. In embodiments, constitutive or inducible expression or production of the PKS gene cluster, or constitutive or inducible expression or production of proteins SEQ ID NO:78-92, or SEQ ID NO:78-85 and 87-92 is contemplated herein.

Other objects and advantages will become apparent to those skilled in the art from a review of the ensuing detailed description, which proceeds with reference to the following illustrative drawings, embodiments, and the attendant claims.

### DESCRIPTION OF THE DRAWINGS

FIG. 1 provides identification of L. reuteri strains by 16S rRNA amplicon sequencing. L. reuteri strains were identified by amplifying and sequencing of the 16S rRNA variable region. Phylogenetic analysis of the 16S rRNA sequences along with other L. reuteri sequences is depicted. Streptococcus pyogenes was included as an outgroup.

FIG. 2 depicts growth profiles of L. reuteri strains in MRS broth. Growth profiles were assessed by growing the strains in MRS broth and determining the CFU counts at different time points. The data shown is representative of 3 independent experiments.

FIG. 3 depicts phylogenetic relationship of L. reuteri strains PTA-126788 and PTA-126787 to other known human L. reuteri strains using 92 core genes. The phylogenetic relationship was explored using UBCG v3.0 and a maximum likelihood tree was inferred using GTR+CAT model. Streptococcus thermophilus and Enterococcus faecalis were used as outgroups.

FIG. 4 depicts quantification of production of D- and L-lactic acid by L. reuteri strains. L- and D-lactic acids were quantified using D-/L-lactic acid (D-L-lactate) Rapid Assay Kit (Megazyme). The data represent the mean±SD from 3 independent experiments.

FIG. 5 depicts ability of L. reuteri strains to undergo autoaggregation. A. Ability to undergo autoaggregation was determined by growing the strains overnight in MRS broth and observing for aggregate formation. B. Autoaggregation was quantified by measuring the OD600 in PBS after incubation for 5 hours and calculating the autoaggregation % as described in the methods section. The data represents the mean±SE of 3 independent experiments.

FIG. 6 depicts ability of L. reuteri strains to produce hydrogen peroxide. Hydrogen peroxide production was assessed by growing the strains on MRS agar supplemented with 0.25 mg/ml of tetramethylbenzidine and 0.01 mg/ml of horseradish peroxidase and observing for color change. Dark blue coloration indicates high production of hydrogen peroxide. The data are representative of 3 independent experiments.

FIG. 7 depicts tolerance of L. reuteri strains to 0.3% bile. The ability of L. reuteri strains to tolerate bile salts was assessed by growing the strains in the presence of 0.3% bile salts for 4 hours and determining the CFU counts at 0 hours and 4 hours after incubation with bile salts. The data represent the mean±SD from 3 independent experiments.

FIG. 8 shows tolerance of L. reuteri strains to acidic pH. The ability of L. reuteri strains to tolerate acidic pH was assessed by growing the strains at pH 2.5 for 3 hours and determining the CFU counts at 0 hours and 3 hours after incubation. The data represent the mean±SD from 3 independent experiments.

FIGS. 9A and 9B provides genome mapping of the (A) L. reuteri strain PTA-126787 and (B) L. reuteri strain PTA-126788 showing regions and positions occupied by different types of prophages as predicted by PhiSpy. The inner circle in each shows the locations of the genome contigs. The prophage locations on the genome are indicated and depicted in the outer circle. Six prophage regions in strain PTA-126787 and eight regions in PTA-126788 were identified and are indicated.

FIG. 10 depicts the protocol for the binge on chronic alcohol murine model utilized in these studies.

FIG. 11 provides the effect of various treatments/feed conditions on gut leak in the binge on chronic alcohol murine model. Animals were fed PF: Pair-fed, AF: Alcohol-fed, AFEP: alcohol-fed+ probiotic combination of L. reuteri strains 3630 and 3632, AFBB: Alcohol-fed+ Blueberry and AFBC: Alcohol-fed+ Broccoli. L. reuteri was administered for 10 days. Mice were acclimated in Lieber-DeCarli liquid control diet and randomized into alcohol fed (AF; Lieber-deCarli ethanol (EtOH) liquid diet) and pair-fed (PF) groups (control diet). AF mice were fed with EtOH liquid diet (5.0%, vol/vol) for 10 days and received 4 g/kg body weight EtOH by gavage at day 5 and day 10. AFEP group was fed with 3×10⁹ CFUs/mice/day of L. reuteri 3630 and 3632 (1:1) ratio for 10 days. FITC-dextran was gavaged and fluorescence measured in the serum to determine gut leak. FITC-dextran was gavaged then fluorescence measured in the serum.

FIGS. 12 A and B depicts the effect of L. reuteri administration for 10 days on the expression of the cytokines (A) IL-6 and (B) TNF-α in fold change in the intestine. PF, pair fed; AF, alcohol fed; AFP, alcohol fed+ compound X; AFPE, alcohol fed+ probiotic combination of L. reuteri strains 3630 and 3632; AFBB, Alcohol-fed+ Blueberry; AFBC, Alcohol-fed+ Broccoli.

FIGS. 13 A and B depicts the effect of L. reuteri administration for 10 days on the expression of the cytokines (A) IL-22 and (B) IL-10 in fold change in the intestine. PF, pair fed; AF, alcohol fed; AFP, alcohol fed+ compound X; AFPE, alcohol fed+ probiotic combination of L. reuteri strains 3630 and 3632; AFBB, Alcohol-fed+ Blueberry; AFBC, Alcohol-fed+ Broccoli.

FIGS. 14 A and B depicts the effect of L. reuteri administration for 10 days on the expression of the cytokines (A) IFN-γ and (B) IL-1β in fold change in the intestine. PF, pair fed; AF, alcohol fed; AFP, alcohol fed+ compound X; AFPE, alcohol fed+ probiotic combination of L. reuteri strains 3630 and 3632; AFBB, Alcohol-fed+ Blueberry; AFBC, Alcohol-fed+ Broccoli.

FIG. 15 depicts the AHR signaling pathway. The inactive form of AHR is localized in the cytosol in a complex composed of HSP90, AIP, p23, and c-SRC. AHR agonists induce conformational changes in AHR that result in its translocation to the nucleus. In the nucleus, AHR interacts with ARNT, and the heterodimer is responsible for the transcription of XRE-containing genes. Notes: (AHR) aryl hydrocarbon receptor, (N) N terminal motif, (C) C terminal motif, (NLS) nuclear localization signal, (bHLH) basic-helix loop helix, (PAS) Per-Arnt-Sim, (Q-rich) glutamine rich, (HSP90) heat shock protein 90, (AIP) AHR-interacting protein, (XRE) xenobiotic responsive elements, (AHRR) AHR repressor, (CYP) cytochrome P450, (IDO) indoleamine 2,3-dioxygenase

FIG. 16 depicts AhR metabolite gene clusters. A comparison of maps of the gene clusters relating to AhR metabolite production and synthesis from several L. reuteri strains is provided. The gene cluster from L. reuteri strain 3632 (LR3632) is compared to each of L. reuteri strains 2010 and R21c.

FIG. 17 provides in vitro data from HepG2-Lucia cells demonstrating dose-dependent AHR activation by fractions from L. reuteri strain 3632 (LR3632) extract enriched for pks metabolite and depicts AhR activator activity of control FICZ versus L. reuteri 3632 pks metabolite (2.5 mg/ml).

FIG. 18 shows AHR activation studies. AHR activator activity was assessed in L. reuteri 3632 supernatant, cell pellet resuspended, and medium. Significant AhR activator activity was identified in the L. reuteri 3632 pellet resuspended. The medium and supernatant had limited to no significant activity.

FIG. 19 provides AhR activation studies and assessment of AhR activator activity. The cell pellet was resuspended in the same volume as the culture supernatant and a direct comparison of ligand presence in the supernatant versus the pellet resusupended was conducted. Again, the activity is demonstrated in the pellet resuspended with little activity in the L. reuteri strain 3632 0.2 μM filtered supernatant.

### DETAILED DESCRIPTION

The present disclosure provides probiotic compositions and methods of use in improving animal health. In embodiments, methods and uses are provided for reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome. The probiotic strains and probiotic compositions, particularly Lactobacillus reuteri strains and compositions thereof, are useful for and have application and activity in methods provided for reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome.

In one embodiment, the invention provides a probiotic composition including at least one isolated Lactobacillus reuteri strain. The at least one Lactobacillus reuteri strain includes at least one of a isolated first Lactobacillus reuteri strain and a isolated second Lactobacillus reuteri strain.

The at least one isolated Lactobacillus reuteri strain may include one Lactobacillus reuteri strain or a combination of two or more Lactobacillus reuteri strains. The Lactobacillus reuteri strains may have been selected for gut adaptation in animals, such as poultry. The Lactobacillus reuteri strains may be been isolated from poultry.

In one embodiment, the isolated first Lactobacillus reuteri has a nucleic acid or amino acid sequence including at least one of SEQ ID NOs: 1-24, 26, and 49-55, sequences having at least 98%, at least 98.5%, at least 99%, or at least 99.5% sequence identity with at least one of SEQ ID NOs: 1-24, 26, and 49-55. In one embodiment, the isolated first Lactobacillus reuteri has a nucleic acid genome sequence including at least one of SEQ ID NOs: 49-55, sequences having at least 98%, at least 98.5%, at least 99%, or at least 99.5% sequence identity with at least one of SEQ ID NOs: 49-55. In one embodiment, the isolated first Lactobacillus reuteri strain has a nucleic acid genome sequence comprising SEQ ID NOs: 49-55, or a sequence having at least 98%, at least 98.5%, at least 99%, or at least 99.5% sequence identity with SEQ ID NOs: 49-55.

In one embodiment, the isolated first Lactobacillus reuteri has a nucleic acid or amino acid sequence including at least one of SEQ ID NOs: 1-24, 26, and 49-55, sequences having at least one nucleic acid or amino acid sequence difference from the sequence of at least one of SEQ ID NOs: 1-24, 26, and 49-55, sequences having one or more nucleic acid or amino acid sequence difference from the sequence of at least one of SEQ ID NOs: 1-24, 26, and 49-55, sequences having at least one nucleic acid or amino acid sequence difference from the sequence of at least one of SEQ ID NOs: 1-24, 26, and 49-55 and further having at least 99% sequence identity with at least one of SEQ ID NOs: 1-24, 26, and 49-55, sequences having at least one nucleic acid or amino acid sequence difference from the sequence of at least one of SEQ ID NOs: 1-24, 26, and 49-55 and further having at least 99% sequence identity with one or more of SEQ ID NOs: 1-24, 26, and 49-55, sequences having at least 98%, at least 98.5%, at least 99%, or at least 99.5% sequence identity with at least one of SEQ ID NOs: 1-24, 26, and 49-55.

In a preferred embodiment, the isolated first Lactobacillus reuteri strain is Lactobacillus reuteri strain 3632. As used herein, “Lactobacillus reuteri strain 3632”, “LR 3632”, and “3632” “ATCC Patent Deposit Number PTA-126788”, “strain PTA-126788”, V and “PTA-126788” may be used interchangeably.

In some embodiments, the isolated second Lactobacillus reuteri strain has a nucleic acid sequence or amino acid sequence including at least one of SEQ ID NOs: 25, 27-43, and 44-48, sequences having at least 98%, at least 98.5%, at least 99%, or at least 99.5% sequence identity with at least one of SEQ ID NOs: 25, 27-43, and 44-48. In embodiments, the isolated second Lactobacillus reuteri strain has a nucleic acid genome sequence comprising SEQ ID NOs: 44-48, or a sequence having at least 98%, at least 98.5%, at least 99%, or at least 99.5% sequence identity with SEQ ID NOs: 44-48.

In one embodiment, the isolated second Lactobacillus reuteri has a nucleic acid or amino acid sequence including at least one of SEQ ID NOs: 25, 27-43, and 44-48, sequences having at least one nucleic acid or amino acid sequence difference from the sequence of at least one of SEQ ID NOs: 25, 27-43, and 44-48, sequences having one or more nucleic acid or amino acid sequence difference from the sequence of at least one of SEQ ID NOs: 25, 27-43, and 44-48, sequences having at least one nucleic acid or amino acid sequence difference from the sequence of at least one of SEQ ID NOs: 25, 27-43, and 44-48 and further having at least 99% sequence identity with at least one of SEQ ID NOs: 25, 27-43, and 44-48, sequences having at least one nucleic acid or amino acid sequence difference from the sequence of at least one of SEQ ID NOs: 25, 27-43, and 44-48 and further having at least 99% sequence identity with one or more of SEQ ID NOs: 25, 27-43, and 44-48, sequences having at least 98%, at least 98.5%, at least 99%, or at least 99.5% sequence identity with at least one of SEQ ID NOs: 25, 27-43, and 44-48.

In a preferred embodiment, the second Lactobacillus reuteri is Lactobacillus reuteri strain 3630. As used herein, “Lactobacillus reuteri strain 3630”, “LR 3630”, “3630”, “ATCC Patent Deposit Number PTA-126787”, “strain PTA-126787” and “PTA-126787” may be used interchangeably.

In a preferred embodiment, the at least one isolated Lactobacillus reuteri strain includes strain 3632 and 3630. In another preferred embodiment, the at least one isolated Lactobacillus reuteri strain is L. reuteri strain 3632, or a strain having a nucleic acid genome sequence including at least one of SEQ ID NOs: 49-55, or a strain having a nucleic acid genome sequence having at least 98%, at least 98.5%, at least 99%, or at least 99.5% sequence identity with at least one of SEQ ID NOs: 49-55. In a preferred embodiment, the at least one isolated Lactobacillus reuteri strain is L. reuteri strain 3632. In another preferred embodiment, the at least one isolated Lactobacillus reuteri strain is L. reuteri strain 3630, or a strain having a nucleic acid genome sequence including at least one of SEQ ID NOs: 44-48, or a strain having a nucleic acid genome sequence having at least 98%, at least 98.5%, at least 99%, or at least 99.5% sequence identity with at least one of SEQ ID NOs: 44-48. In a preferred embodiment, the at least one isolated Lactobacillus reuteri strain is L. reuteri strain 3630.

In an embodiment, the at least one isolated Lactobacillus reuteri strain is a combination of Lactobacillus reuteri strain 3632 and strain 3630. In an embodiment, a combination of two or more Lactobacillus reuteri strains is a combination of strain 3632 and strain 3630. In an embodiment, a combination of two or more Lactobacillus reuteri strains is a combination of strain PTA-126788 and PTA-126787.

In an embodiment, the isolated strains of the present disclosure are not genetically modified by recombinant or genetically engineered means. In an embodiment, the strains of use and application in accordance with the disclosure are genetically modified by recombinant or genetically engineered means. In an embodiment, the isolated strains of the present disclosure are genetically modified by recombinant or genetically engineered means, such as to delete or inactivate the tetW gene or prevent tetW protein production. Deletion or inactivation may include modifications wherein the strain(s) are not genetically modified, such as for example wherein one or more of the strains are cultured for selection and isolation of a variant or mutant wherein expression or activity of tetW is altered, reduced or terminated or absent. Such a variant or mutant wherein expression or activity of tetW is altered, reduced or terminated or absent is altered but has not been genetically modified by recombinant or genetically engineered means.

In a preferred embodiment, the at least one isolated Lactobacillus reuteri strain is selected from strain 3632 and 3630. In a preferred embodiment, the composition, particularly a probiotic composition, comprises a combination of isolated Lactobacillus reuteri strains 3632 and 3630. In a preferred embodiment, the composition, particularly an immunogenic probiotic composition, comprises a combination of isolated Lactobacillus strains PTA-126788 and PTA-126787. In a preferred embodiment, the composition, particularly a probiotic composition, comprises a combination of isolated Lactobacillus strains PTA-126788 and PTA-126787 or a Lactobacillus strain having at least 98% or 99% amino acid or nucleic acid identity to strain PTA-126788 and isolated Lactobacillus strain PTA-126788 and PTA-126787 or a Lactobacillus strain having at least 99% amino acid or nucleic acid identity to strain PTA-126787. In a preferred embodiment, the composition, particularly a probiotic composition, comprises a combination of isolated Lactobacillus strains PTA-126788 and PTA-126787 or a Lactobacillus strain having at least 98% or at least 99% amino acid or nucleic acid identity to strain PTA-126788 and isolated Lactobacillus strains PTA-126788 and PTA-126787 or a Lactobacillus strain having at least 98% or at least 99% amino acid or nucleic acid identity to strain PTA-126787, wherein the strains each and/or together have probiotic activity or capability. In a preferred embodiment, the composition, particularly a probiotic composition, comprises a combination of isolated Lactobacillus strains PTA-126788 and PTA-126787 or a Lactobacillus strain having at least 98% or at least 99% amino acid or nucleic acid identity to strain PTA-126788 and isolated Lactobacillus strains PTA-126788 and PTA-126787 or a Lactobacillus strain having at least 98% or at least 99% amino acid or nucleic acid identity to strain PTA-126787, wherein the strains each and/or together have probiotic activity or capability and capability and activity to improve animal health.

In a preferred embodiment, the at least one isolated Lactobacillus reuteri strain includes strain 3632 and 3630. In a preferred embodiment, the at least one isolated Lactobacillus reuteri strain is selected from strain 3632 and 3630. In a preferred embodiment, the composition, particularly a probiotic composition, comprises a combination of isolated Lactobacillus reuteri strains 3632 and 3630. In a preferred embodiment, the composition, particularly a probiotic composition, comprises a combination of isolated Lactobacillus strains PTA-126788 and PTA-126787. In a preferred embodiment, the composition, particularly a probiotic composition, comprises a combination of isolated Lactobacillus strains PTA-126788 and PTA-126787 or a Lactobacillus strain having at least 99% amino acid or nucleic acid identity to strain PTA-126788 and isolated Lactobacillus strain PTA-126788 and PTA-126787 or a Lactobacillus strain having at least 99% amino acid or nucleic acid identity to strain PTA-126787. In a preferred embodiment, the composition, particularly a probiotic composition, comprises a combination of isolated Lactobacillus strains PTA-126788 and PTA-126787 or a Lactobacillus strain having at least 99% amino acid or nucleic acid identity to strain PTA-126788 and isolated Lactobacillus strains PTA-126788 and PTA-126787 or a Lactobacillus strain having at least 99% amino acid or nucleic acid identity to strain PTA-126787, wherein the strains each and/or together have probiotic activity or capability. In a preferred embodiment, the composition, particularly a probiotic composition, comprises a combination of isolated Lactobacillus strains PTA-126788 and PTA-126787 or a Lactobacillus strain having at least 99% amino acid or nucleic acid identity to strain PTA-126788 and isolated Lactobacillus strains PTA-126788 and PTA-126787 or a Lactobacillus strain having at least 99% amino acid or nucleic acid identity to strain PTA-126787, wherein the strains each and/or together have probiotic activity or capability and capability and activity to improve animal health.

In an embodiment, the strains each and/or together have activity or capability for reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome in an animal.

In some embodiments, compositions disclosed herein include an isolated first Lactobacillus reuteri strain and an isolated second Lactobacillus reuteri strain at a ratio of approximately 0.75-1.5:1. In a preferred embodiment, the composition includes about equal amounts of the isolated first Lactobacillus reuteri strain and the isolated second Lactobacillus reuteri strain, or approximately 1:1. In an embodiment, the composition includes about equal amounts, such as equal amounts measured as CFU/kg or CFU/ml of the composition, of the isolated first Lactobacillus reuteri strain and the isolated second Lactobacillus reuteri strain, or approximately 1:1.

The compositions disclosed herein can be formulated as animal feed, feed additive, food ingredient, food additive, medicament additive or ingredient, water additive, water-mixed additive, consumable solution, consumable spray additive, consumable solid, consumable gel, injection, or combinations thereof. In one embodiment, the composition includes water.

In some embodiments, the compositions disclosed include the isolated first Lactobacillus reuteri strain in an amount of about 10²-10⁸ CFU/kg of the composition, about 10⁶-10⁸ CFU/kg of the composition, about 10⁴-10⁷ CFU/kg of the composition, about 10³-10⁵ CFU/kg of the composition, about 10² CFU/kg of the composition, about 10³ CFU/kg of the composition, about 10⁶ CFU/kg of the composition, about 10 CFU/kg of the composition, or about 10⁸ CFU/kg of the composition. In some embodiments, the compositions disclosed herein includes the isolated first Lactobacillus reuteri strain in an amount of about 10²-10⁸ CFU/ml of the composition, about 10⁶-10⁸ CFU/ml of the composition, about 10⁴-10⁷ CFU/ml of the composition, about 10³-10⁵ CFU/ml of the composition, about 10³ CFU/ml of the composition, about 10⁴ CFU/ml of the composition, about 10⁵ CFU/ml of the composition, about 10⁶ CFU/ml of the composition, about 10 CFU/ml of the composition, or about 10⁸ CFU/ml of the composition.

In some embodiments, the compositions disclosed herein includes the isolated second Lactobacillus reuteri strain in an amount of about 10²-10⁸ CFU/kg of the composition, about 10⁶-10⁸ CFU/kg of the composition, about 10⁴-10 CFU/kg of the composition, about 10³-10⁵ CFU/kg of the composition, about 10² CFU/kg of the composition, about 10³ CFU/kg of the composition, about 10⁶ CFU/kg of the composition, about 10 CFU/kg of the composition, or about 10⁸ CFU/kg of the composition. In some embodiments, the compositions disclosed herein includes the isolated second Lactobacillus reuteri strain in an amount of about 10²-10⁸ CFU/ml of the composition, about 10⁶-10⁸ CFU/ml of the composition, about 10⁴-10 CFU/ml of the composition, about 10³-10⁵ CFU/ml of the composition, about 10³ CFU/ml of the composition, about 10⁴ CFU/ml of the composition, about 10⁵ CFU/ml of the composition, about 10⁶ CFU/ml of the composition, about 10 CFU/ml of the composition, or about 10⁸ CFU/ml of the composition.

The present disclosure also provides methods of increasing animal health, wherein the method includes administering an effective amount of the composition to an animal. In accordance with the invention, methods are provided for reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome.

In an embodiment, the Lactobacillus strains, particularly strain PTA-126787 and strain PTA-126788, provide an enzymatic profile as follows:

| | | PTA- | PTA- |
|---|---|---|---|
| Enzyme assayed | Substrate | 126787 | 126788 |
| Alkaline phosphatase | 2-naphthyl phosphate | − | − |
| Esterase (C 4) | 2-naphthyl butyrate | + | + |
| Esterase Lipase (C 8) | 2-naphthyl capylate | +/− | +/− |
| Lipase (C 14) | 2-naphthyl myristate | − | − |
| Leucine arylamidase | L-leucyl-2-naphthylamide | +++ | +++ |
| Valine arylamidase | L-valyl-2-naphthylamide | ++ | ++ |
| Cystine arylamidase | L-cystyl-2-naphthylamide | + | + |
| Trypsin | N-benzoyl-DL-argine-2- | − | − |
| | naphthylamide | | |
| α-chymotrypsin | N-glutaryl-phenylalanine-2- | − | − |
| | naphthylamide | | |
| Acid phosphatase | 2-naphthyl phosphate | +++ | +++ |
| Naphthol-AS BI- | Naphthol-AS-BI-phosphate | + | + |
| phosphohydrolase α- | 6-Br-2-naphthyl-αD- | +++ | +++ |
| galactosidase | galactopyranoside | | |
| β-galactosidase | 2-naphthyl-βD- | +++ | +++ |
| | galactopyranoside | | |
| β-glucuronidase | Naphthol-AS-BI-βD- | + | + |
| | glucuronide | | |
| α-glucosidase | 2-naphthyl-αD- | + | + |
| | glucopyranoside | | |
| β-glucosidase | 6-Br-2-naphthyl-βD- | − | − |
| | glucopyranoside | | |
| N-acetyl-β- | 1-naphthyl-N-acetyl-βD- | +/− | +/− |
| | glucosaminide | | |
| glucosaminidase α- | 6-Br-2-naphthyl-αD- | − | − |
| mannosidase | mannopyranoside | | |
| α-fucosidase | 2-naphthyl-αL- | − | − |
| | fucopyranoside | | |

+, positive enzymatic reaction;
−, negative enzymatic reaction

In an embodiment, the Lactobacillus strains, particularly strain PTA-126787 and strain PTA 126788, provide the following enzymes, particularly providing the enzymes in a significant amount:

| Enzyme assayed | Substrate |
|---|---|
| Esterase (C 4) | 2-naphthyl butyrate |
| Leucine arylamidase | L-leucyl-2-naphthylamide |
| Valine arylamidase | L-valyl-2-naphthylamide |
| Cystine arylamidase | L-cystyl-2-naphthylamide |
| Acid phosphatase | 2-naphthyl phosphate |
| Naphthol-AS BI- | Naphthol-AS-BI-phosphate |
| phosphohydrolase | |
| α-galactosidase | 6-Br-2-naphthyl-αD-galactopyranoside |
| β-galactosidase | 2-naphthyl-βD-galactopyranoside |
| β-glucuronidase | Naphthol-AS-BI-βD-glucuronide |
| α-glucosidase | 2-naphthyl-αD-glucopyranoside |

The present disclosure also provides methods of increasing animal health, wherein the method includes administering an effective amount of the composition to an animal. Methods are provided for reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome

The composition disclosed herein and above increases animal health by providing positive health benefits when administered to an animal, as compared to an animal that has not been administered the composition. As used herein, “animal” includes bird, a human, or a non-human mammal. Specific examples of birds include poultry such as chickens or turkey. Specific examples of animal include chickens, turkey, dogs, cats, cattle and swine. The chicken may be a broiler chicken or egg-laying or egg-producing chicken. The animal may be a human. The animal may be a non-human mammal.

Positive health benefits include decreasing feed conversion ratio, increasing weight, increasing lean body mass, decreasing pathogen-associated lesion formation in the gastrointestinal tract, decreasing colonization of pathogens, reducing inflammation, and decreasing mortality rate. Positive health benefits described, demonstrated and provided herein include reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome.

Altered or compromised intestinal permeability and intestinal dysbiosis are important and relevant to various clinical conditions and symptoms and problems. The most direct causes of altered or increased intestinal permeability include: chronic inflammatory states, such as IBD and celiac disease; other diseases that cause intestinal injury, such as HIV/AIDS; chemotherapy and radiation therapies that degrade the intestinal mucosa; chronic overuse of alcohol or NSAIDs, such as aspirin and ibuprofen; food allergies that cause an immune response to certain foods. Intestinal permeability is a recognized feature of several inflammatory and autoimmune diseases affecting the digestive system, including inflammatory bowel disease, Crohn's disease and celiac disease.

There are both pro-inflammatory and anti-inflammatory molecules or cytokines. The pro-inflammatory cytokines are secreted from Th1 cells, CD4⁺ cells, macrophages, and dendritic cells. They are characterized by production of several Interleukins (IL), IL-1, IL-2, IL-12, IL-7, IL-18, IFN-γ, and TNF-α. The key pro-inflammatory cytokines are IL-1, IL-6, and TNF-α. Pro-inflammatory chemokines are produced by cells primarily to recruit leukocytes to the sites of infection or injury. They are crucial for coordinating cell mediated immune response and play a critical role in modulating the immune system. Pro-inflammatory cytokines generally regulate growth, cell activation, differentiation, and homing of the immune cells to the sites of infection with the aim to control and eradicate intracellular pathogens. IL-1 is subdivided in IL-1α and IL-1β. IL-1β is potent pro-inflammatory cytokine, induced mainly by lymphocytes, macrophages, and monocytes in response to microbial molecules. The anti-inflammatory cytokines are a series of immunoregulatory molecules that control the proinflammatory cytokine response. Anti-inflammatory cytokines include IL-10, which inhibits cytokine production and mononuclear cell function, IL-12, which activates NK cells, IL-22, which stimulates cell survival and proliferation, and TGF-β, which Inhibits T and B cell proliferation, Anti-inflammatory interleukins include interleukin (IL)-1 receptor antagonist, 11-4, IL-6, IL-10, IL-13, IL-19 and IL-35.

The studies set out and provided herein demonstrate that administration of the probiotic compositions described results in reduced intestinal permeability, particularly in an established leaky gut animal model. The studies set out and provided herein further demonstrate that administration of the probiotic compositions described results in reduced levels of pro-inflammatory cytokines, including IL-6, TNF-α, IFN-γ, and IL-1β. The studies further show that administration of the probiotic compositions provided herein results in increased levels of the anti-inflammatory molecules or cytokines IL-22 and IL-10.

In some embodiments, the compositions disclosed herein reduce pro-inflammatory molecules or cytokines by at least 10%, at least 20%, at least 25%, at least 50%, at least 60%, at least 80%. In some embodiments, the compositions disclosed herein reduce pro-inflammatory molecules or cytokines by at least 1 fold, 2 fold, 3 fold, 4 fold. In some embodiments, the compositions disclosed herein reduce pro-inflammatory molecules or cytokines IL-6, TNF-α, IFN-γ, and/or IL-1β by at least 10%, at least 20%, at least 25%, at least 50%, at least 60%, at least 80%. In some embodiments, the compositions disclosed herein reduce pro-inflammatory molecules or cytokines IL-6, TNF-α, IFN-γ, and/or IL-1β by at least 1 fold, 2 fold, 3 fold, 4 fold, 6 fold, S fold, 10 fold. In some embodiments, the compositions disclosed herein increase anti-inflammatory molecules or cytokines by at least 10%, at least 20%, at least 25%, at least 50%, at least 60%, at least 80%. In some embodiments, the compositions disclosed herein increase anti-inflammatory molecules or cytokines by at least 1 fold, 2 fold, 3 fold, 4 fold, 6 fold, S fold, 10 fold. In some embodiments, the compositions disclosed herein increase anti-inflammatory molecules or cytokines IL-22 and/or IL-10 by at least 10%, at least 20%, at least 25%, at least 50%, at least 60%, at least 80%. In some embodiments, the compositions disclosed herein increase anti-inflammatory molecules or cytokines IL-22 and/or IL-10 by at least 1 fold, 2 fold, 3 fold, 4 fold, 6 fold, 8 fold, 10 fold.

In some embodiments, the compositions disclosed herein reduce intestinal permeability or leaky gut by at least 5%, at least 10%, at least 20%, at least 25%, at least 30%, at least 40%, at least 50%, at least 60%, at least 70%, or at least 80%. In some embodiments, the compositions disclosed herein reduce intestinal permeability or leaky gut by at least 1 fold, 2 fold, 3 fold, 4 fold, 6 fold, 8 fold, 10 fold.

The composition of one or more L reuteri strain may be combined with one or more other or anti-inflammatory agent, molecule or cytokine or immune modulator. Immune modulators may include cytokines, hormones, antibodies which modulate, including to particularly reduce or alleviate the immune response or inflammatory response. The composition of one or more L reuteri strain may be combined with one or more anti-inflammatory drug or immune suppressants/immune modulator, including the one or more drug or modifier described herein. The composition of one or more L reuteri strain may be combined with one or more anti-inflammatory, nonsteroidal anti-inflammatory drug (NSAID), steroid, biologic, antibiotic, or anti-diarrheal agent, including as described above. The composition of one or more L reuteri strain may be combined with an anti-inflammatory cytokine such as IL-10, IL-12 or IL-22. The composition of one or more L reuteri strain may be combined with an IL-1 inhibitor, such as an IL-1 receptor antagonist.

As used herein, pathogen includes Salmonella, Clostridium, Campylobacter, Staphylococcus, Streptococcus, and E. coli bacterium. Further examples of pathogens include Salmonella typhimurium, Salmonella infantis, Salmonella Hadar, Salmonella enteritidis, Salmonella Newport, Salmonella Kentucky, Clostridium perfringens, Staphylococcus aureus, Streptococcus uberis, Streptococcus suis, Escherichia coli, Campylobacter jejuni, and Fusobacterium necrophorum.

The compositions may be administered orally, parentally, nasally, or mucosally. Parental administration includes subcutaneous, intramuscular and intravenous administration.

In some aspects, administration includes feeding the poultry, or spraying onto the poultry. In other aspects, administration includes on ovo administration or in ovo administration. In an embodiment, administered comprises in ovo administration. In an embodiment, administered comprises spray administration. In an embodiment, administered comprises immersion, intranasal, intramammary, topical, or inhalation.

In some aspects the animal is vaccinated in conjunction with administration. The animal may be vaccinated prior to administration of the compositions disclosed herein. The animal may be vaccinated with an coccidiosis vaccine. Coccidiosis vaccines are known in the art, for example, COCCIVAC.

In some embodiments, administration is by way of injection or infusion. In one embodiment, the composition is administered to a cow by way of intra-mammary infusion.

In an embodiment of the method(s), the method does not comprise administration of an antibiotic.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation. When the animal is a bird the treatment may be administered in ovo or by spray inhalation.

Compositions may include a carrier in which the bacterium or any such other components is suspended or dissolved. Such carrier(s) may be any solvent or solid or encapsulated in a material that is non-toxic to the inoculated animal and compatible with the organism. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is preferably presented in the form of an aerosol. A dye may be added to the compositions hereof, including to facilitate checking or confirming whether an animal has ingested or breathed in the composition.

When administering to animals, including humans or farm animals, administration may include orally or by injection. Oral administration can include by bolus, tablet or paste, or as a powder or solution in feed, food, or drinking water. Administration may be by ingestion. The method of administration will often depend on the species being feed or administered, the numbers of animals being fed or administered, and other factors such as the handling facilities available and the risk of stress for the animal.

The dosages required will vary and need be an amount sufficient to induce a response or to effect a biological or phenotypic change or response expected or desired. Routine experimentation will establish the required amount. Increasing amounts or multiple dosages may be implemented and used as needed.

The strains disclosed herein demonstrate certain phenotypic properties. Without wishing to be bound by theory, it is believed that these phenotypic properties at least contribute to increasing animal health.

In some embodiments, the isolated strains secrete at least one of cyclic dipeptides (cyclo(his-phe) and cyclo (phe-pro), short chain fatty acids (2-hydroxy-3-methylvalerate and alpha-hydroxyisocaproate), betaine, dimethylglycine, essential amino acids (e.g., allo-threonine, phosphothreonine, histidine, lysine, phenylalanine, tryptophan, leucine, isoleucine, and cysteine s-sulfate), nucleotides (e.g., adenosine 5′-monophosphate (AMP), uridine 5′-monophosphate (UMP), cytidine 5′-monophosphate (5′-CMP), and cytidine 2′3′-cyclicmonophosphate), myo-inositol, and indolin-2-one. Some of the aforementioned molecules provide beneficial characteristics to the host, including increased weight, pro-inflammatory effects, and antibiotic effects.

In some embodiments, the composition including the isolated first Lactobacillus reuteri strain (strain 3632) and the isolated second Lactobacillus reuteri strain (strain 3630) in combination, will secrete certain beneficial molecules in larger quantities than when individually cultured. For example, with combinations and cocultures of strains 3630 and 3632, increased levels of each of the following are provided or secreted (as determined from culture supernatants): dimethylglycine, allo-threonine, 1-methyl-4-imidazoleacetate, 4-imidazoleacetate, lysine, N6-methyllysine, N6, N6-dimethyllysine, 5-aminovalerate, and tyrosine, 4-hydroxyphenylpyruvate, indolacetate, and gamma-glutamylglutamine, glucose 6-phosphate, 4-hydroxyl-2-oxoglutaric acid, and myo-inositol, Uridine 5′-monophosphate (UMP), Cytidine 5′-monophosphate (5′-CMP), 3′-5′-uridylyluridine, O-sulfo-L-tyrosine, indole 3 acetamide, indolin-2-one and daidzein. In particular, when the strains 3630 and 3632 are combined in cultures or are grown together, significant and synergistic amounts (more than just additive) of some beneficial molecules are present or secreted. In particular embodiments, significant amounts of the molecules 4-hydroxyphenylpyruvate and glucose 6-phosphate are secreted or present with combinations of strains 3630 and 3632, or with compositions including a mix of about equal amounts of strains 3630 and 3632.

In some embodiments, the animal administered the composition exhibits a shift in the microbiome content of the gastrointestinal tract. For example, there may be an increase in the amount of bacteroidaceae bacteria in the gut of an animal that has been administered the composition described herein, as compared to an animal that was not administered the composition.

In embodiments of the present invention, the composition includes a combination of two isolated Lactobacillus reuteri strains.

Without wishing to be bound by a particular theory, it is believed that the probiotic composition of the present disclosure can alleviate leaky gut, reduce intestinal permeability, and reduce intestinal inflammation. In this regard, and in particular, the composition is believed to correct or address the altered and compromised intestinal microbial community with chronic alcohol consumption and intestinal diseases and inflammation and for methods that provide beneficial bacteria and/or molecules to the gastrointestinal tract of an animal and improve animal health, particularly in animals consuming alcohol on a chronic or binge basis, animals with leaky gut, increased intestinal permeability and intestinal-derived or intestinal-associated inflammation. Intestinal-derived or intestinal-associated inflammation can include inflammatory bowel diseases (IBD), including ulcerative colitis and Crohn's disease.

Anti-inflammatory drugs or immune suppressants/immune modulators are often the first step in the treatment of intestinal inflammation, including associated with IBD, such as ulcerative colitis, typically for mild to moderate disease. Time-limited courses of corticosteroids are also used to induce remission. Nonsteroidal anti-inflammatory drugs (NSAIDs) have been used for the treatment of inflammation, including intestinal inflammatory diseases. NSAIDs include non-prescription drugs acetylsalicylate (aspirin), ibuprofen (Advil, Motrin TB) and naproxen sodium (Aleve, Naprosyn) and prescription NSAIDs such as etodolac (Lodine) and diclofenac (Voltaren). Anti-inflammatories include aminosalicylates, such as mesalamine (Delzicol, Rowasa, others), balsalazide (Colazal) and olsalazine (Dipentum). Steroids are anti-inflammatory or immunosuppressants agents that can be prescribed and utilized in treatment. Examples include glucocotricosteroids or corticosteroids such as prednisone, cortisone and methylprednisolone. Time-limited courses of corticosteroids are also used to reduce inflammation. Some examples of immunosuppressant drugs include azathioprine (Azasan, Imuran), mercaptopurine (Purinethol, Purixan) and methotrexate (Trexall).

Biologics—genetically engineered proteins which target a specific aspect or part of the immune system and act as immunosuppressants or neutralize proteins causing inflammation—are an increasingly important component in treatment of significant inflammatory conditions and IBD. Biologics include abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz), vedolizumab (Entyvio), ustekinumab (Stelara), and risankizumab (Skyrizi). Adalimumab, etanercept, infliximab, golimumab and certolizumab target tumor necrosis factor (TNF). Rituximab is effective against B cells. Anakinra (interleukin-1 receptor antagonist (IL-1Ra)) blocks the action of the proinflammatory master cytokine interleukin-1 (IL-1), a master cytokine. Abatacept targets T cells. Adalimumab, etanercept, infliximab, golimumab and certolizumab target TNF (Lis K et al (2014) Arch Med Sci 10(6):1175-1185). Rituximab depletes B cells. Orally delivered agents also known as “small molecules” available for intestinal inflammation and IBD treatment include tofacitinib (Xeljanz), upadacitinib (Rinvoq) and ozanimod (Zeposia).

Antibiotics may be used in addition to other medications or when infection is a concern. Frequently prescribed antibiotics include ciprofloxacin (Cipro) and metronidazole (Flagyl). Anti-diarrheal medications, such as loperamide (Imodium), and/or fiber supplements may also be utilized.

In embodiments of the invention, including the methods provided, the probiotic compositions described herein may be combined, co-administered, or integrated in and with treatment protocols and agents utilized to reduce inflammation or modulate the immune system. In embodiments of the compositions or methods, the L reuteri strains 3632 and 3630 are combined or administered in conjunction with, including before, after, or in series, including in altering series of administration, treatment protocols and agents utilized to reduce inflammation or modulate the immune system.

The agents or treatment protocols to be combined or included may include one or more anti-inflammatory drugs or immune suppressants/immune modulators described above. The agents or treatment protocols to be combined or included may be selected from one or more anti-inflammatory, Nonsteroidal anti-inflammatory drug (NSAID), steroid, biologic, antibiotic, or anti-diarrheal agent, including as described above.

Cytokine profiling is typically done using ELISA using lung/tracheal/intestinal homogenates or serum samples. In the absence of reagents needed for ELISA (antibodies for detection of various cytokines) cytokine profiling is done using qRT-PCR on mRNA isolated from RNAlater preserved samples. For example, tissue samples can be collected in RNAlater for cytokine mRNA isolation and/or examination either qualitatively and/or quantitatively by qRT-PCR. Relevant and proinflammatory cytokines include, but are not limited to IL-1, IL-2, IL-12, IL-17, IL-18, IFN-γ, and TNF-α. The key pro-inflammatory cytokines are IL-1, TL-6, and TNF-α. Relevant and anti-inflammatory cytokines include IL-10, IL-22 and IL-12.

In an embodiment of the invention, a composition is provided for alleviating the effects of chronic alcohol consumption and increased intestinal permeability in an animal. In an embodiment of the invention, a composition is provided for reducing susceptibility to an infectious agent or pathogen, including such as bacteria, in an animal with or under conditions of chronic and/or binge alcohol consumption. In an embodiment of the invention, a composition is provided for reducing intestinal inflammation associated with gastrointestinal disease including or such as inflammatory bowel disease (IBD) or celiac disease in an animal or subject. In an embodiment of the invention, a composition is provided for alleviating the symptoms and clinical pathologies, such as pain and inflammation, associated with gastrointestinal disease including or such as inflammatory bowel disease (IBD), Crohn's disease, ulcerative colitis, or celiac disease in an animal or subject. In an embodiment, a composition is provided comprising Lactobacillus strains, particularly strain 3630 (PTA-126787) and strain 3632 (PTA-126788). In an embodiment, a composition is provided comprising Lactobacillus strains, particularly strain 3630 (PTA-126787) and strain 3632 (PTA-126788) in about a 1:1 strain ratio based on CFU/strain per kg, or per mg in the composition. In an embodiment, a composition is provided comprising Lactobacillus strains, particularly strain 3630 (PTA-126787) and strain 3632 (PTA-126788) in about a 1:1 strain ratio based on CFU/strain per kg, or per mg or per g of the animal or subject being administered the composition.

Binge-on-chronic alcohol feeding in animal models shifts gut microbiota diversity and functional capacity. In embodiments of the invention, compositions and methods are provided for alleviating or preventing intestinal dysbiosis under conditions of and in instances of chronic and/or binge alcohol consumption in an animal. In an embodiment, compositions and methods are provided for maintaining or restoring gut microbiota diversity with or under conditions of chronic and/or binge alcohol consumption in an animal. In an embodiment, compositions and methods are provided for protecting the gut and/or intestinal system from harm, alteration or disease with or under conditions of chronic and/or binge alcohol consumption in an animal. In an embodiment, compositions and methods are provided for preventing or alleviating leaky gut with or under conditions of chronic and/or binge alcohol consumption in an animal.

In an embodiment, compositions and methods are provided for reducing susceptibility to an infectious agent or pathogen, including such as bacteria, in an animal with or under conditions of chronic and/or binge alcohol consumption. In an embodiment, compositions and methods are provided for reducing susceptibility to an infectious agent or pathogen, including such as bacteria, in an animal with or under conditions of gastrointestinal disease or inflammation, including inflammatory bowel disease (IBD), Crohn's disease or celiac disease. In an embodiment, compositions and methods are provided for increasing resistance or maintaining protection against an infectious agent or pathogen, including such as bacteria, in an animal with or under conditions of chronic and/or binge alcohol consumption. As used herein, pathogen includes a bacteria that infects an animal including animals selected from a bird, human or non human animal, including non human animals such as chickens, turkey, dogs, cats, cattle, and swine. Pathogen includes Klebsiella, Salmonella, Clostridium, Campylobacter, Staphylococcus, Streptococcus, and E. coli bacterium.

In one embodiment, the compositions and methods are provided for reducing intestinal inflammation associated with gastrointestinal disease including or such as inflammatory bowel disease (IBD) or celiac disease in an animal or subject. Inflammatory bowel disease includes Crohn's disease and ulcerative colitis. In one embodiment, the compositions and methods are provided for reducing inflammation in the gut or intestine in an animal or subject.

In one embodiment, the compositions and methods are provided for alleviating the symptoms and clinical pathologies, such as pain and inflammation, associated with gastrointestinal disease including or such as inflammatory bowel disease (IBD), Crohn's disease, ulcerative colitis, or celiac disease in an animal or subject.

The methods include further administering one or more anti-inflammatory agent, molecule or cytokine or immunomodulatory to an animal or subject. The method includes further administering, including in combination in the composition, one or more anti-inflammatory agent, molecule or cytokine or immunomodulatory to an animal or subject.

In certain aspects of the disclosure, the isolated Lactobacillus reuteri strain exist as isolated and biologically pure cultures. It will be appreciated by one of skill in the art, that an isolated and biologically pure culture of a particular Lactobacillus reuteri strain, denotes that said culture is substantially free (within scientific reason) of other living organisms and contains only the individual Lactobacillus reuteri strain in question. The culture can contain varying concentrations of said isolated Lactobacillus reuteri strain. The present disclosure notes that isolated and biologically pure microbes often necessarily differ from less pure or impure materials.

In embodiments of the present invention, the composition includes one or more isolated Lactobacillus reuteri strains. In embodiments of the present invention, the composition includes a combination of two isolated Lactobacillus reuteri strains. In some embodiments of the present invention, the composition includes a combination of two or more isolated Lactobacillus reuteri strains. In embodiments of the present invention, the composition includes one or more isolated Lactobacillus reuteri strain and one or more other bacterial strain. In embodiments of the present invention, the composition includes two isolated Lactobacillus reuteri strains and one or more other bacterial strain. In embodiments of the present invention, the composition includes the Lactobacillus reuteri strain denoted 3632 and one or more other bacterial strain. In embodiments of the present invention, the composition includes the Lactobacillus reuteri strain denoted 3630 and one or more other bacterial strain. The other or one or more other bacterial strain may be another Lactobacillus strain, a Bacillus strain, a Salmonella strain.

In certain aspects of the disclosure, the isolated Lactobacillus reuteri strain exists as isolated and biologically pure cultures. It is appreciated by one of skill in the art, that an isolated and biologically pure culture of a particular Lactobacillus reuteri strain, denotes that said culture is substantially free (within scientific reason) of other living organisms and contains only the individual Lactobacillus reuteri strain in question. The culture can contain varying concentrations of said isolated Lactobacillus reuteri strain. The present disclosure notes that isolated and biologically pure microbes often necessarily differ from less pure or impure materials.

In some embodiments of the present invention, the composition includes a combination of two isolated Lactobacillus reuteri strains. In some embodiments of the present invention, the composition includes a combination of three isolated Lactobacillus reuteri strains.

### Definitions

As used herein, “isolated” means that the subject isolate has been separated from at least one of the materials with which it is associated in a particular environment, for example, its natural environment.

Thus, an “isolate” does not exist in its naturally occurring environment; rather, it is through the various techniques known in the art that the microbe has been removed from its natural setting and placed into a non-naturally occurring state of existence. Thus, the isolated strain or isolated microbe may exist as, for example, a biologically pure culture in association with an acceptable carrier.

As used herein, “individual isolates” should be taken to mean a composition, or culture, comprising a predominance of a single species, or strain, of microorganism, following separation from one or more other microorganisms. The phrase should not be taken to indicate the extent to which the microorganism has been isolated or purified. However, “individual isolates” can include substantially only one species, or strain, of microorganism.

As used herein, the term “bacterial consortia”, “bacterial consortium”, “microbial consortia”, or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest (e.g. increasing vaccine efficacy). The community may comprise two or more species, or strains of a species (eg., Lactobacillus reuteri strains 3632 and 3630), of microbes. In some instances, the microbes coexist within the community symbiotically.

As used herein, the terms “colonize” and “colonization” include “temporarily colonize” and “temporary colonization”.

As used herein, “probiotic” refers to a substantially pure microbe (i.e., a single isolate) or a mixture of desired microbes, and may also include any additional components (e.g., carrier) that can be administered to an animal to provide a beneficial health effect. Probiotics or microbial compositions of the invention may be administered with an agent or carrier to allow the microbes to survive the environment of the gastrointestinal tract, i.e., to resist low pH and to grow in the gastrointestinal environment.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2nd Ed. CRC Press. 504 pg.); and E. W. Martin (1970. Remington's Pharmaceutical Sciences. 17th Ed. Mack Pub. Co.).

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “subject” includes bird, poultry, a human, or a non-human mammal. Specific examples include chickens, turkey, dogs, cats, cattle, and swine. The chicken may be a broiler chicken, egg-laying or egg-producing chicken. As used herein, the term “poultry” includes domestic fowl, such as chickens, turkeys, ducks, quail, and geese.

As used herein, the term “immunogenic” means than an agent is capable of eliciting an immune response, including an innate, humoral, or cellular immune response, and both. “Immunogenic” includes “immunomodulatory”. An immunogenic composition is a composition that elicits an innate, humoral, or cellular immune response, or both.

As used herein, the term “immune response” includes a response by a subject that involves generation of antibodies that bind to an antigen (i.e., an antibody response). This does not exclude generation of a cell-mediated response.

By “stimulating” is meant directly or indirectly increasing the level and/or functional activity of a target system (e.g., immune system). In certain embodiments, “stimulation” or “stimulating” means that a desired/selected response is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than if the vaccine had been used alone.

Any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

Throughout this specification, quantities are defined by ranges, and by lower and upper boundaries of ranges. Each lower boundary can be combined with each upper boundary to define a range. The lower and upper boundaries should each be taken as a separate element. Two lower boundaries or two upper boundaries may be combined to define a range.

### Deposit Information

Lactobacillus reuteri strain “3630” was deposited on 19 Jun. 2020 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-126787.

Lactobacillus reuteri strain “3632” was deposited on 19 Jun. 2020 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-126788.

Access to the deposits is available during the pendency of this application to persons determined by the Commissioner of Patents and Trademarks to be entitled thereto under 37 C.F.R. § 1.14 and 35 U.S.C. § 122. Upon allowance of any embodiments in this application, all restrictions on the availability to the public of the variety is irrevocably removed.

The deposits is maintained in the ATCC depository, which is a public depository, for a period of 30 years, or 5 years after the most recent request, or for the effective life of the patent, whichever is longer, and is replaced if a deposit becomes nonviable during that period.

The following embodiments and aspects illustrate and are not intended to limit scope of the present invention. Instead, these embodiments and aspects provide guidance to any skilled artisan on how to prepare and use compositions and methods taught by the present invention, where such skilled artisans will appreciate that modifications may be made without departing from the spirit and scope of the invention. The present disclosure is exemplified by specific embodiments below.

### EMBODIMENTS

1. A composition having at least one isolated Lactobacillus reuteri strain, wherein said composition increases animal health when an effective amount is administered to an animal, as compared to an animal not administered the composition.

2. A composition having at least one of an isolated first Lactobacillus reuteri strain and an isolated second Lactobacillus reuteri strain, wherein said composition increases animal health when an effective amount is administered to an animal, as compared to an animal not administered the composition.

3. A composition having at least one isolated Lactobacillus reuteri strain, wherein said composition increases animal health, including in alleviating the effects of chronic alcohol consumption, leaky gut, increased intestinal permeability and/or intestinal inflammation, when an effective amount is administered to an animal, as compared to an animal not administered the composition.

4. A composition having at least one of an isolated first Lactobacillus reuteri strain and an isolated second Lactobacillus reuteri strain, wherein said composition increases animal health, including in alleviating the effects of chronic alcohol consumption, leaky gut, increased intestinal permeability and/or intestinal inflammation, when an effective amount is administered to an animal, as compared to an animal not administered the composition.

5. The composition of embodiments 2 or 4, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

6. The composition of embodiment 2, 4 or 5, wherein:
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

7. The composition of embodiment 2, 4, 5 or 6, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

8. A composition for use in increasing animal health, including alleviating the effects of chronic alcohol consumption, leaky gut, increased intestinal permeability and/or intestinal inflammation, when an effective amount is administered to an animal, as compared to an animal not administered the composition and wherein the composition comprises at least one of an isolated first Lactobacillus reuteri strain and an isolated second Lactobacillus reuteri strain.

9. A composition for use in reducing intestinal permeability or leaky gut in an animal comprising at least one of an isolated first Lactobacillus reuteri strain and an isolated second Lactobacillus reuteri strain.

10. A composition for use in reducing inflammation, including intestinal-derived or intestinal-associated inflammation in an animal comprising at least one of an isolated first Lactobacillus reuteri strain and an isolated second Lactobacillus reuteri strain.

11. A composition for use in reducing intestinal inflammation associated with gastrointestinal disease including or such as inflammatory bowel disease (IBD), Crohn's disease, or celiac disease in an animal comprising at least one of an isolated first Lactobacillus reuteri strain and an isolated second Lactobacillus reuteri strain.

12. The composition for use of any of embodiments 8, 9, 10 or 11, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

13. The composition for use of any of embodiments 8, 9, 10, 11 or 12, wherein:
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

14. The composition for use of any of embodiments 8, 9, 10, 11, 12 or 13, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

15. A composition for reducing the production or expression of one or more proinflammatory cytokine in the intestine of an animal comprising at least one of an isolated first Lactobacillus reuteri strain and an isolated second Lactobacillus reuteri strain.

16. A composition for reducing the production or expression of one or more proinflammatory cytokine selected from IL-6, TNF-α, IFN-γ and IL-1, including IL-1β, in the intestine of an animal comprising at least one of an isolated first Lactobacillus reuteri strain and an isolated second Lactobacillus reuteri strain.

17. A composition for increasing the production or expression of one or more anti-inflammatory cytokine in the intestine of an animal comprising at least one of an isolated first Lactobacillus reuteri strain and an isolated second Lactobacillus reuteri strain.

18. A composition for increasing the production or expression of one or more anti-inflammatory cytokine selected from IL-10 and IL-22 in the intestine of an animal comprising at least one of an isolated first Lactobacillus reuteri strain and an isolated second Lactobacillus reuteri strain.

19. The composition of any of embodiments 15, 16, 17 or 18, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

20. The composition for use of any of embodiments 15, 16, 17, 18 or 19, wherein:
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

21. The composition for use of any of embodiments 15, 16, 17, 18 19 or 20, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

22. The composition or composition for use of any of embodiments 1-21, wherein at least one of the first Lactobacillus strain and a second Lactobacillus strain secrete at least one of cyclic dipeptides, short chain fatty acids, betaine, dimethylglycine, essential amino acids, nucleotides, myo-inositol, and indolin-2-one.

23. The composition according to any one of embodiments 1-22, wherein the composition comprises a ratio of isolated first Lactobacillus reuteri strain to isolated second Lactobacillus reuteri strain of 0.75-1.5:1.

24. The composition according to any one of embodiments 1-23, wherein the composition comprises about equal amounts of the isolated first Lactobacillus reuteri strain and the isolated second Lactobacillus reuteri strain.

25. The composition according to any one of embodiments 1-24, wherein the composition is formulated as animal feed, feed additive, food ingredient, water additive, water-mixed additive, consumable solution, consumable spray additive, consumable solid, consumable gel, injection, or combinations thereof.

26. The composition according to embodiment 25, wherein the composition comprises animal feed.

27. The composition according to any of embodiments 1-26, wherein the composition comprises the isolated first Lactobacillus reuteri strain in an amount of about 10²-10⁸ CFU/kg of the composition, about 10⁴-10⁷ CFU/kg of the composition, or about 10³-10⁵ CFU/kg of the composition.

28. The composition according to any one of embodiments 1-26, wherein the composition comprises isolated second Lactobacillus reuteri strain in an amount of about 10²-10⁸ CFU/kg of the composition, about 10⁴-10⁷ CFU/kg of the composition, or about 10³-10⁵ CFU/kg of the composition.

29. The composition according to any one of embodiments 1-27, wherein the composition comprises the isolated first Lactobacillus reuteri strain in an amount of about 10⁷ CFU/kg of the composition.

30. The composition according to any one of embodiments 1-28, wherein the composition comprises isolated second Lactobacillus reuteri strain in an amount of about 10⁷ CFU/kg of the composition.

31. The composition according to any one of embodiments 1-30, wherein the animal is bird, poultry, a human, or a non-human mammal.

32. A method for reducing intestinal permeability, alleviating alcohol induced or disease related leaky gut syndrome, alleviating the intestinal and systemic effects of chronic alcohol consumption, including intestinal dysbiosis, and reducing inflammation, including intestinal-derived or intestinal-associated inflammation, all and any of which include administration of an effective amount of an immunogenic probiotic composition comprising at least one of an isolated first Lactobacillus reuteri strain and an isolated second Lactobacillus reuteri strain.

33. A method for reducing intestinal permeability, alleviating alcohol induced or disease related leaky gut syndrome, alleviating the intestinal and systemic effects of chronic alcohol consumption, including intestinal dysbiosis, and reducing inflammation, including intestinal-derived or intestinal-associated inflammation, all and any of which include administration of an effective amount of an immunogenic probiotic composition comprising a combination of two or at least two Lactobacillus reuteri strains, including at least one of an isolated first Lactobacillus reuteri strain and an isolated second Lactobacillus reuteri strain.

34. The method of embodiments 32 or 33, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

35. The method of embodiments 32, 33 or 34, wherein:
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

36. The method of embodiments 32, 33, 34 or 35, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

37. The method of any of embodiments 32-36 comprising administering an effective amount of a probiotic composition comprising at least one Lactobacillus reuteri strain and one or more anti-inflammatory agent, molecule or cytokine to a subject or animal.

38. The method of embodiment 37, wherein the one or more anti-inflammatory agent, molecule or cytokine is selected from one or more anti-inflammatory, nonsteroidal anti-inflammatory drug (NSAID), steroid, biologic, antibiotic, or anti-diarrheal agent.

39. The method of embodiment 38, wherein the biologic targets a specific aspect or part of the immune system and acts as an immunosuppressant or neutralizes one or more protein causing inflammation.

40. The method of any of embodiments 32-36, further comprising administering an effective amount of a prebiotic.

41. The method of embodiment 40, wherein the prebiotic is selected from mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

42. A method of alleviating or modulating increased intestinal permeability or leaky gut in a subject comprising administering an effective amount of a probiotic composition comprising at least one of an isolated first Lactobacillus reuteri strain and an isolated second Lactobacillus reuteri strain.

43. The method of embodiment 42, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

44. The method of embodiments 42 or 43, wherein
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

45. The method of embodiments 42, 43 or 44, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

46. The method of any of embodiments 42-45, comprising administering an effective amount of a probiotic composition comprising at least one Lactobacillus reuteri strain and one or anti-inflammatory agent, molecule or cytokine.

47. The method of embodiment 46, wherein the one or more anti-inflammatory agent, molecule or cytokine is selected from one or more anti-inflammatory, nonsteroidal anti-inflammatory drug (NSAID), steroid, biologic, antibiotic, or anti-diarrheal agent.

48. The method of embodiment 47, wherein the biologic targets a specific aspect or part of the immune system and acts as an immunosuppressant or neutralizes one or more protein causing inflammation.

49. The method of any of embodiments 42-45, further comprising administering an effective amount of a prebiotic.

50. The method of embodiment 49, wherein the prebiotic is selected from mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

51. A method of alleviating or modulating increased intestinal permeability or leaky gut in a subject comprising administering an effective amount of a probiotic composition comprising administering an effective amount of an probiotic composition comprising at least one Lactobacillus reuteri strain wherein the at least one Lactobacillus reuteri strain comprises at least one sequence selected from SEQ ID NO:1-55 and sequences having at least 98%, at least 98.5%, at least 99%, or at least 99.5% sequence identity with at least one of SEQ ID NO: 1-55.

52. The method of embodiment 51, wherein the at least one Lactobacillus reuteri strain includes at least one of: a nucleic acid sequence having at least 95%, at least 96%, at least 97%, at least 98%, or at least 99% sequence identity with SEQ ID NO:26, a nucleic acid sequence having at least 95%, at least 96%, at least 97%, at least 98%, or at least 99% sequence identity with SEQ ID NO: 1, a nucleic acid sequence having at least 95%, at least 96%, at least 97%, at least 98%, or at least 99% sequence identity with SEQ ID NO: 3, and a nucleic acid that encodes for an amino acid sequence having at least 95%, at least 96%, at least 97%, at least 98%, or at least 99% sequence identity with SEQ ID NO: 8.

53. The method of embodiment 51, wherein the at least one Lactobacillus reuteri strain includes at least one of: a nucleic acid sequence having at least 95%, at least 96%, at least 97%, at least 98%, or at least 99% sequence identity with SEQ ID NO:25, a nucleic acid sequence having at least 95%, at least 96%, at least 97%, at least 98%, or at least 99% sequence identity with SEQ ID NO: 27, a nucleic acid sequence having at least 95%, at least 96%, at least 97%, at least 98%, or at least 99% sequence identity with SEQ ID NO: 28, and a nucleic acid sequence having at least 95%, at least 96%, at least 97%, at least 98%, or at least 99% sequence identity with SEQ ID NO: 29.

54. The method of any of embodiments 51, 52 or 53, wherein the at least one Lactobacillus reuteri strain has a nucleic acid or amino acid sequence including at least one of SEQ ID NOs: 1-24, 26, and 49-55, sequences having at least one nucleic acid or amino acid sequence difference from the sequence of at least one of SEQ ID NOs: 1-24, 26, and 49-55, sequences having one or more nucleic acid or amino acid sequence difference from the sequence of at least one of SEQ ID NOs: 1-24, 26, and 49-55, sequences having at least one nucleic acid or amino acid sequence difference from the sequence of at least one of SEQ ID NOs: 1-24, 26, and 49-55 and further having at least 99% sequence identity with at least one of SEQ ID NOs: 1-24, 26, and 49-55, sequences having at least one nucleic acid or amino acid sequence difference from the sequence of at least one of SEQ ID NOs: 1-24, 26, and 49-55 and further having at least 99% sequence identity with one or more of SEQ ID NOs: 1-24, 26, and 49-55, sequences having at least 98%, at least 98.5%, at least 99%, or at least 99.5% sequence identity with at least one of SEQ ID NOs: 1-24, 26, and 49-55.

55. The method of embodiment 51, 52, 53 or 54, wherein the at least one Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55.

56. The method of embodiment 51, 52, 53, 54 or 55, wherein the at least one Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788.

57. The method of embodiment 56, wherein the at least one Lactobacillus reuteri strain is Lactobacillus reuteri strain 3632, which corresponds to ATCC Patent Deposit Number PTA-126788.

58. The method of embodiment 51, wherein the at least one Lactobacillus reuteri strain has a nucleic acid sequence or amino acid sequence including at least one of SEQ ID NOs: 25, 27-43, and 44-48, sequences having at least 98%, at least 98.5%, at least 99%, or at least 99.5% sequence identity with at least one of SEQ ID NOs: 25, 27-43, and 44-48.

59. The method of embodiment 51 or 58, wherein the at least one Lactobacillus reuteri strain has a nucleic acid or amino acid sequence including at least one of SEQ ID NOs: 25, 27-43, and 44-48, sequences having at least one nucleic acid or amino acid sequence difference from the sequence of at least one of SEQ ID NOs: 25, 27-43, and 44-48, sequences having one or more nucleic acid or amino acid sequence difference from the sequence of at least one of SEQ ID NOs: 25, 27-43, and 44-48, sequences having at least one nucleic acid or amino acid sequence difference from the sequence of at least one of SEQ ID NOs: 25, 27-43, and 44-48 and further having at least 99% sequence identity with at least one of SEQ ID NOs: 25, 27-43, and 44-48, sequences having at least one nucleic acid or amino acid sequence difference from the sequence of at least one of SEQ ID NOs: 25, 27-43, and 44-48 and further having at least 99% sequence identity with one or more of SEQ ID NOs: 25, 27-43, and 44-48, sequences having at least 98%, at least 98.5%, at least 99%, or at least 99.5% sequence identity with at least one of SEQ ID NOs: 25, 27-43, and 44-48.

60. The method of embodiment 51, 58 or 59, wherein the at least one Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

61. The method of embodiment 58, 59 or 60, wherein the at least one Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

62. The method of embodiment 51 or 61, wherein the at least one Lactobacillus reuteri strain is Lactobacillus reuteri strain 3630, which corresponds to ATCC Patent Deposit Number PTA-126787.

63. The method of embodiment 51, wherein the at least one Lactobacillus strain comprises at least one of an isolated first Lactobacillus reuteri strain and an isolated second Lactobacillus reuteri strain.

64. The method of embodiment 63, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

65. The method of embodiments 63 or 64, wherein
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

66. The method of embodiments 63, 64 or 65, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

67. The method of any of embodiments 51-66, comprising administering an effective amount of a probiotic composition comprising at least one Lactobacillus reuteri strain, and an anti-inflammatory agent, molecule or cytokine, to a subject or animal.

68. The method of embodiment 67, wherein the one or more anti-inflammatory agent, molecule or cytokine is selected from one or more anti-inflammatory, nonsteroidal anti-inflammatory drug (NSAID), steroid, biologic, antibiotic, or anti-diarrheal agent.

69. The method of embodiment 68, wherein the biologic targets a specific aspect or part of the immune system and acts as an immunosuppressant or neutralizes one or more protein causing inflammation.

70. The method of any of embodiments 51-66, further comprising administering an effective amount of a prebiotic.

71. The method of embodiment 70, wherein the prebiotic is selected from mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

72. A method of reducing intestinal permeability in a subject comprising administering an effective amount of a probiotic composition comprising at least one Lactobacillus reuteri strain.

73. The method of embodiment 72, wherein the probiotic composition comprises at least one Lactobacillus reuteri strain wherein the at least one Lactobacillus reuteri strain comprises at least one sequence selected from SEQ ID NO:1-55 and sequences having at least 98%, at least 98.5%, at least 99%, or at least 99.5% sequence identity with at least one of SEQ ID NO:1-55.

74. The method of embodiment 72 or 73, wherein the probiotic composition comprises at least one Lactobacillus reuteri strain having a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55.

75. The method of embodiment 72, wherein the probiotic composition comprises at least one of an isolated first Lactobacillus reuteri strain and an isolated second Lactobacillus reuteri strain.

76. The method of embodiment 72 or 75, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

77. The method of embodiments 72, 75 or 76, wherein
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

78. The method of embodiments 72, 75, 76 or 77, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

79. The method of embodiments 72-78, comprising administering an effective amount of a probiotic composition comprising at least one Lactobacillus reuteri strain and one or more an anti-inflammatory agent, molecule or cytokine.

80. The method of embodiment 79, wherein the one or more an anti-inflammatory agent, molecule or cytokine is selected from one or more anti-inflammatory, nonsteroidal anti-inflammatory drug (NSAID), steroid, biologic, antibiotic, or anti-diarrheal agent.

81. The method of embodiment 80, wherein the biologic targets a specific aspect or part of the immune system and acts as an immunosuppressant or neutralizes one or more protein causing inflammation.

82. The method of any of embodiments 72-78, further comprising administering an effective amount of a prebiotic.

83. The method of 82, wherein the prebiotic is selected from mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

84. A method for reducing intestinal inflammation associated with gastrointestinal disease including or selected from inflammatory bowel disease (IBD) or celiac disease in an animal comprising administering to the animal a composition comprising at least one of an isolated first Lactobacillus reuteri strain and an isolated second Lactobacillus reuteri strain.

85. The method of embodiment 84, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

86. The method of embodiments 84 or 85, wherein
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

87. The method of embodiments 84, 85 or 86, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

88. The method of embodiments 84-87, wherein the inflammatory bowel disease (IBD) is Crohn's disease or ulcerative colitis.

89. A method for reducing the production or expression of one or more proinflammatory cytokine in the intestine of an animal comprising administering to the animal a composition comprising at least one of an isolated first Lactobacillus reuteri strain and an isolated second Lactobacillus reuteri strain.

90. A method for reducing the production or expression of one or more proinflammatory cytokine selected from IL-6, TNF-α, IFN-γ and IL-1, including IL-1β, in the intestine of an animal comprising administering to the animal a composition comprising at least one of an isolated first Lactobacillus reuteri strain and an isolated second Lactobacillus reuteri strain.

91. A method for increasing the production or expression of one or more anti-inflammatory cytokine in the intestine of an animal comprising administering to the animal a composition comprising at least one of an isolated first Lactobacillus reuteri strain and an isolated second Lactobacillus reuteri strain.

92. A method for increasing the production or expression of one or more anti-inflammatory cytokine selected from IL-10 and IL-22 in the intestine of an animal comprising administering to the animal a composition comprising at least one of an isolated first Lactobacillus reuteri strain and an isolated second Lactobacillus reuteri strain.

93. The method of any of embodiments 89, 90, 91 or 92, wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and
(b) the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48.

94. The method of embodiments 89, 90, 91, 92 or 93, wherein
(a) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, and
(b) the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787.

95. The method of any of embodiments 89-94, wherein the isolated first Lactobacillus reuteri strain is Lactobacillus reuteri strain 3632, which corresponds to ATCC Patent Deposit Number PTA-126788 and the isolated second Lactobacillus reuteri is Lactobacillus reuteri strain 3630, which corresponds to ATCC Patent Deposit Number PTA-126787.

96. The method of any of embodiments 84-95, wherein the at least one or the two Lactobacillus reuteri strains are administered prior to one or more anti-inflammatory agent, molecule or cytokine or immunomodulatory agent.

97. The method of embodiment 96, wherein the one or more an anti-inflammatory agent, molecule or cytokine is selected from one or more anti-inflammatory, nonsteroidal anti-inflammatory drug (NSAID), steroid, biologic, antibiotic, or anti-diarrheal agent.

98. The method of embodiment 97, wherein the biologic targets a specific aspect or part of the immune system and acts as an immunosuppressant or neutralizes one or more protein causing inflammation.

99. The method of any of embodiments 84-95, further comprising administering an effective amount of a prebiotic.

100. The method of embodiment 99, wherein the prebiotic is selected from mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

101. The method of any of embodiments 37, 46, 67, 78 or 96, wherein the at least one or the two Lactobacillus reuteri strains are administered prior to and in conjunction one or more anti-inflammatory agent, molecule or cytokine or immunomodulatory.

102. The method of any of embodiments 37, 46, 67, 78 or 96, wherein the at least one or the two Lactobacillus reuteri strains are administered prior to, in conjunction with, and following one or more anti-inflammatory agent, molecule or cytokine or immunomodulatory.

103. The method of any of embodiments 37, 46, 67, 78 or 96, wherein the at least one or the two Lactobacillus reuteri strains are administered in combination with or shortly following one or more anti-inflammatory agent, molecule or cytokine or immunomodulator.

104. The method of any of embodiments 37, 46, 67, 78 or 96, wherein one or more anti-inflammatory agent, molecule or cytokine or immunomodulator is administered as a single dose or multiple doses.

105. The method of any of embodiments 37, 46, 67, 78 or 96, wherein the at least one or the two Lactobacillus reuteri strains are administered prior to and/or between and/or in combination with multiple doses of one or more anti-inflammatory agent, molecule or cytokine or immunomodulator.

106. The method according to any one of embodiments 32-101, wherein the composition comprises a ratio of isolated first Lactobacillus reuteri strain to isolated second Lactobacillus reuteri strain of 0.75-1.5:1.

107. The method according to any one of embodiments 32-101, wherein the composition comprises about equal amounts of the isolated first Lactobacillus reuteri strain and the isolated second Lactobacillus reuteri strain.

108. The method according to any one of embodiments 32-101, wherein the composition is formulated as animal feed, feed additive, food ingredient, water additive, water-mixed additive, consumable solution, consumable spray additive, consumable solid, consumable gel, injection, or combinations thereof.

109. The method according to embodiment 108, wherein the composition comprises animal feed.

110. The method according to any of embodiments 32-101, wherein the composition comprises the isolated first Lactobacillus reuteri strain in an amount of about 10²-10⁸ CFU/kg of the composition, about 10⁴-10⁷ CFU/kg of the composition, or about 10³-10⁵ CFU/kg of the composition.

111. The method according to any one of embodiments 32-101, wherein the composition comprises isolated second Lactobacillus reuteri strain in an amount of about 10²-10⁸ CFU/kg of the composition, about 10⁴-10⁷ CFU/kg of the composition, or about 10³-10 CFU/kg of the composition.

112. The method according to any one of embodiments 32-101 wherein the composition comprises the isolated first Lactobacillus reuteri strain in an amount of about 10 CFU/kg of the composition.

113. The method according to any one of embodiments 32-101, wherein the composition comprises isolated second Lactobacillus reuteri strain in an amount of about 10 CFU/kg of the composition.

114. The method according to any one of embodiments 32-101, wherein the animal is bird, poultry, a human, or a non-human mammal.

115. The composition in accordance with any of embodiments 8-31 or 32-114 wherein the composition is administered orally or by injection.

116. The composition in accordance with embodiment 8-31, 32-114 or 115, wherein the composition is administered orally, optionally by food administration or as a food or feed additive.

117. The composition in accordance with embodiment 8-31, 32-114 or 115, wherein the composition is administered orally, optionally by direct ingestion in a powder, liquid, emulsion or pill.

118. The composition in accordance with any of embodiments 8-31 or 32-114 wherein the composition is administered wherein administered by in ovo administration.

119. The composition in accordance with any of embodiments 8-31 or 32-114 wherein the composition is administered by spray administration.

120. The method of embodiment 32-114, wherein the animal is a bird, a human, or a non-human mammal.

121. The method of embodiment 120, wherein the bird is poultry.

122. The method of embodiment 121, wherein the poultry is a chicken.

123. The method of embodiment 121, wherein the poultry is an egg-producing chicken.

124. The method of embodiment 120, wherein the animal is a farm animal or food production animal.

125. The method of embodiment 120, wherein the animal is a human.

126. The method of embodiment 120, wherein the animal is a horse, dog or cat.

127. The method of any of embodiments 32-114, wherein the animal administered the composition exhibits a shift in the microbiome content of gastrointestinal tract.

128. A biosynthetic gene cluster (BGC), particularly a polyketide synthase (PKS) BGC, capable of producing an AhR-activating metabolite.

129. The biosynthetic gene cluster (BGC) of embodiment 128, wherein the BGC is an isolated polyketide synthase (PKS) BGC of Lactobacillus reuteri strain 3632 is provided.

130. The biosynthetic gene cluster (BGC) of embodiment 128 or 129, wherein the PKS BGC comprises the nucleic acid set out in SEQ ID NO: 77.

131. The biosynthetic gene cluster (BGC) of embodiment 128, 129 or 130, wherein the PKS BGC comprises nucleic acid encoding one or more polypeptide selected from SEQ ID NOs: 78-92.

132. The biosynthetic gene cluster (BGC) of embodiment 128, 129 or 130, wherein the PKS BGC comprises nucleic acid encoding one or more polypeptide selected from SEQ ID NOs: 78-85 and 87-92.

133. The biosynthetic gene cluster (BGC) of embodiment 128, 129 or 130, wherein the PKS BGC comprises nucleic acid encoding the polypeptides SEQ ID NOs: 78-85 and 87-92.

134. An Ahr-metabolite synthesizing cluster of genes encoding proteins SEQ ID NO: 78-92.

135. An Ahr-metabolite synthesizing cluster of genes encoding proteins SEQ ID NO: 78-85 and SEQ ID NO: 86-92.

136. A plasmid comprising nucleic acid encoding one or more polypeptide selected from SEQ ID NOs: 78-92.

137. The plasmid of embodiment 123, comprising nucleic acid encoding one or more polypeptide selected from SEQ ID NOs: 78-85 and 87-92.

138. The plasmid of embodiment 123, comprising nucleic acid encoding the polypeptides SEQ ID NOs: 78-85 and 87-92.

139. The plasmid of any of embodiments 123, 124 or 125, wherein the encoded polypeptides are capable of synthesizing an Ahr-metabolite.

140. A plasmid comprising an Ahr-metabolite synthesizing cluster of genes encoding proteins SEQ ID NO: 78-92.

141. A plasmid comprising an Ahr-metabolite synthesizing cluster of genes encoding proteins SEQ ID NO: 78-85 and SEQ ID NO: 86-92.

142. The cluster of genes or plasmid of any of embodiments 134-141, wherein the Ahr-metabolite is capable of increasing IL-22 levels in the intestine.

143. The cluster of genes or plasmid of any of embodiments 134-141, wherein the Ahr-metabolite is capable of ameliorating or reducing intestinal inflammation.

144. The cluster of genes or plasmid of any of embodiments 134-141, wherein the Ahr-metabolite is relevant for and capable of maintaining intestinal barrier integrity.

145. The cluster of genes or plasmid of any of embodiments 134-141, wherein the Ahr-metabolite is relevant for and capable of reducing or alleviating increased intestinal permeability.

146. A method for expressing or producing an Ahr-metabolite via controlled expression of the PKS gene cluster encoding proteins SEQ ID NO:78-85 and 87-92.

147. A method for expressing or producing an Ahr-metabolite via controlled expression of the PKS gene cluster encoding proteins SEQ ID NO:78-92.

148. A method for expressing or producing an Ahr-metabolite via inducible expression of the PKS gene cluster encoding proteins SEQ ID NO:78-85 and 87-92.

149. A method for expressing or producing an Ahr-metabolite via inducible expression of the PKS gene cluster encoding proteins SEQ ID NO:78-92.

150. The method of any of embodiments 146-149, wherein the Ahr-metabolite is expressed or produced in Lactobacillus reuteri bacteria.

The present disclosure may be better understood with reference to the examples, set forth below. The following examples are put forth so as to provide those of ordinary skill in the art with a complete disclosure and description of how the compounds, compositions, and/or methods claimed herein are made, evaluated and conducted, and are intended to be purely exemplary and are not intended to limit the disclosure. It is appreciated that other embodiments and uses are apparent to those skilled in the art and that the invention is not limited to these specific illustrative examples or preferred embodiments.

### Example 1

### Isolation and Characterization of L. reuteri 3630 and 3632

L. reuteri 3630 and 3632 were isolated from chicken cecum. Morphology of these strains include opaque, circular colonies with slight whitish center. The LR 3630 colonies have whitish pigmentation and the LR 3632 colonies include dull orange pigmentation. These strains are non-spore forming

The strains are sequenced by PacBio sequencing. 3632 contained 7 contigs and yield a total estimated genome size of 2.4 Mb and LR 3630 contained 5 contigs yielding an estimated genome size of 2.4 Mb. Phylogenetic relationships of the genomes are explored with UBCG v3.0 using default settings. This software tool employs a set of 92 single-copy core genes commonly present in all bacterial genomes. These genes then are aligned and concatenated within UBCG using default parameters. The estimation of robustness of the nodes is done through the gene support index (GSI), defined as the number of individual gene trees, out of the total genes used, that present the same node. A maximum-likelihood phylogenetic tree is inferred using FastTree v.2.1.10 with the GTR+CAT model. LR strains 3632 and 3630 isolates show closest relationship to L. reuteri.

### Example 2

### Antiviral Effect of L. reuteri on Porcine Reproductive and Respiratory Syndrome (PRRSV)

The effect of LR cells and LR supernatant (3630 and 3632) are tested on MARC-145 (Monkey Kidney) cells in a prevention and therapeutic context, in connection with PRRSV. Virus: GFP-PRRSV, MOI of 1.0; Bacteria: Stock (10⁶ cells/mL).

MARC-145 cells are prepared 2-3 days prior to study. The cells are treated with undiluted and 2-fold serially diluted Bacteria/extract up to 1:32 (1:64) before or after PRRSV infection. In a prevention context, Lactobacillus cells or culture supernatant are added first and virus is added 2-4 hrs later. In a therapeutic context, virus is added first and Lactobacillus cells or culture supernatant is added next, 10 min or 1 hr post infection. The PRRSV inhibitory effect is analyzed by GFP expression and CPE up to 72 hpi.

Both live Lactobacillus reuteri cells show more than 50% inhibitory effect on PRRSV at 1:16 to 1:32 dilutions in prevention setting. Similar inhibition (30-40%) was shown for both Lactobacillus reuteri supernatants in prevention setting. Both live Lactobacillus reuteri cells less efficiently inhibited PRRSV but both supernatants more efficiently inhibited virus replication in therapeutic setting (data not shown).

### Example 3

### In Silico, In Vitro and In Vivo Safety Evaluation of Limosilactobacillus reuteri Strains for Potential Probiotic Applications

The last two decades have witnessed a tremendous growth in probiotics and in the numbers of publications on their potential health benefits. Owing to their distinguishing beneficial effects and long history of safe use, species belonging to the Lactobacillus genus are among the most widely used probiotic species in human food and dietary supplements and are finding increased use in animal feed. Here, we isolated, identified, and evaluated the safety of two novel Lactobacillus reuteri (L. reuteri) isolates, ATCC PTA-126787 & ATCC PTA-126788. More specifically, we sequenced the genomes of these two L. reuteri strains using the PacBio sequencing platform. Using a combination of biochemical and genetic methods, we identified the two strains as belonging to L. reuteri species.

Detailed in silico analyses showed that the two strains do not encode for any known genetic sequences of concern for human or animal health. In vitro assays confirmed that the strains are susceptible to clinically relevant antibiotics and do not produce potentially harmful by-products such as biogenic amines. In vitro bile and acid tolerance studies demonstrated that the two strains have similar survival profiles as the commercial L. reuteri probiotic strain DSM 17938. Most importantly, daily administration of the two probiotic strains to broiler chickens in drinking water for 26 days did not induce any adverse effect, clinical disease, or histopathological lesions, supporting the safety of the strains in an in vivo avian model. All together, these data provide in silico, in vitro and in vivo evidence of the safety of the two novel candidates for potential probiotic applications in humans as well as animals.

The term “probiotic” was derived from “pro” (Latin, means “for”) and “bios” (Greek, means “life”) and thereby means “for life”. Probiotics are defined as “live microorganisms that, when administered in adequate amounts, confer a benefit on the host” (1). In recent years, there has been an unprecedented growth in the application of probiotics to support health and well-being. Often consumed as dietary supplements, nutraceuticals or as part of functional foods, probiotics are associated with many health benefits in the form of promoting gut barrier function, including studies on their potential to prevent and/or treat gastrointestinal diseases, inhibit pathogenic bacteria, and favourably modulate gut bacteria, the immune system, and host metabolism (2-5). Several species of microorganisms are used as probiotics and the lactic acid bacteria belonging to the Lactobacillus genus, first described in 1901 (6), are among the most commonly used and well-studied probiotic bacteria with a long history of safe use (7).

Limosilactobacillus reuteri (L. reuteri), a member of the Lactobacillus genus, are Gram positive, non-spore forming, non-motile bacteria, which are naturally adapted to survive under low pH, bile-rich, and microaerophilic to strictly anaerobic gastrointestinal environments (8). German microbiologist Gerhard Reuter first isolated L. reuteri from human fecal and intestinal samples and classified it as L. fermentum biotype II (9); later, Kandler et al., (1980) identified L. reuteri as a distinct species (10). L. reuteri is considered one of the few true autochthonous lactobacilli present frequently in the gastrointestinal tract of all vertebrates, including humans, monkeys, chicken, turkeys, doves, pigs, dogs, lambs, cattle and rodents (11, 12). L. reuteri strains are often known to produce reuterin (a bacteriocin with antimicrobial properties), cobalamin and folate, exclude or inhibit pathogens, modulate immune response, and enhance gut barrier function (13-17). Several clinical studies have been published on the efficacy of L. reuteri in treating gastrointestinal disorders such as infantile colic, regurgitation, functional constipation, abdominal pain, and necrotizing enterocolitis (17-22). L. reuteri was used in sourdough bread in 1980, and was introduced into human functional foods as a starter in the production of a special drink called “BRA (stands for Bifidobacterium, reuteri and Acidophilus)” and a fermented milk called “BRA fil” in 1991 in Sweden (23). Since then, L. reuteri strains, such as DSM 17938 and RC-14, have been widely used as a part of many commercially available dietary supplements and functional foods (17).

Lactic acid bacteria are known for their safety and are one of the probiotic microbial types with the longest history of safe use (7). The L. reuteri species is usually considered safe for human and animal consumption due to the facts that they have been used as part of fermented foods for more than 30 years, they are normal inhabitants of the human and animal gut microflora, and they have regulatory stature in the United States and the European Union. Indeed, several strains belonging to the L. reuteri species were notified to the United States Food and Drug Administration (FDA), including three strains as “Generally Regarded As Safe (GRAS)” for use in specific foods and two strains as new dietary ingredients; the FDA cited no objections to such strains (24-28). Moreover the L. reuteri species was granted “Qualified Presumption of Safety (QPS)” status and is considered safe for use in, or as a source of food for, human and animal consumption by European Food Safety Agency (EFSA) (24-26, 29, 30). A growing number of clinical studies have repeatedly confirmed the safety of L. reuteri not only in healthy individuals but also in immunocompromised individuals such as those positive for HIV (31, 32).

Despite the prior safe use of a probiotic genus and species, the survival properties, efficacy, and safety of probiotics are evaluated on a strain-specific basis. Hence, screening for such properties for every new strain is required before any new probiotic candidate is accepted for human and animal consumption. Hence, various regulatory agencies and experts have established comprehensive recommended guidelines for efficacy and safety assessment of new probiotic candidates (33-35). These guidelines encompass a series of in vitro, in silico and in vivo studies, of which genomics is considered a powerful tool for rapid screening of probiotic candidates for safety.

Genomic characterizations are instrumental in selecting a safe and efficacious probiotic strain. Safety assessment begins with the correct identification of the probiotic candidate and this is important for both scientific and regulatory reasons. Genomic approaches offer high resolution identification of strains by comparing those with other well-characterized, safe, and efficacious probiotic strains. Comparative genomics studies further help to understand the molecular basis of probiotic efficacy, as well as the survival and adaptation of these probiotic strains in the gastrointestinal tract. Most importantly, genomic analyses allow for rapid screening of probiotic candidates for genes encoding antimicrobial resistance, virulence factors, toxins, and biogenic amines, facilitating better understanding of the safety of the probiotic strain of interest. Finally, genome-based analyses also help to investigate the stability of probiotic strains.

The goal of this study was to provide in silico, in vitro and in vivo evidence to support the safety of L. reuteri ATCC PTA-126787 & ATCC PTA-126788 (hereafter referred to as PTA-126787 and PTA-126788) for their use as probiotics in humans as well as animals. More specifically, the strains were identified using a combination of biochemical, 16S rRNA and whole-genome sequencing analyses. The genomes were screened for potential genes encoding antimicrobial resistance, toxins, virulence factors and other harmful metabolites. In silico data were further confirmed using in vitro experiments. The strains were finally analyzed for safety using the broiler chicken as an in vivo model.

### Materials and Methods

### Bacterial Strains and Culture Conditions

The L. reuteri strains described in this study were routinely propagated on Lactobacilli de Man Rogosa Sharpe (MRS, BD Difco) medium anaerobically at 37° C. L. reuteri strain DSM 17938 was used as a reference strain for biochemical identification, D- and L-lactate production, autoaggregation, resistance to bile salts and acidic pH assays. L. reuteri strain ATCC 23272 was used as a reference strain for growth kinetics, autoaggregation, biogenic amine production and D- and L-lactate production assays. L. acidophilus strain ATCC 4356 was used as a reference strain for growth kinetics assay.

### Molecular Identification

The strains were identified using 16S rRNA sequencing. Briefly, L. reuteri strains were grown in Lactobacilli MRS broth overnight for 14-16 hours under anaerobic conditions at 37° C. One hundred microliters of the culture was pelleted by centrifugation and resuspended in 50 μL of nuclease-free water. The resuspended culture was heated at 98° C. for 10 minutes. The debris were pelleted by brief centrifugation and the supernatant was used as a template for PCR. The 16S rRNA gene was amplified by PCR using 3 μL of the DNA template and universal primers 16S rRNA gene F, 5′-AGAGTTTGATCCTGGCTCAG-3′ (SEQ ID NO:56) and 16S rRNA gene R, 5′-CTTGTGCGGGCCCCCGTCAATTC-3′ (SEQ ID NO:57). The amplicons were PCR purified using the QIAquick PCR Purification Kit (Qiagen Inc.) following manufacturer's instructions and sequenced by Sanger sequencing by GenScript. The sequences were then searched against the NCBI nucleotide collection (nr/nt) database using the BLAST algorithm.

### Biochemical Identification

The strains were profiled for enzymatic activity and carbohydrate fermentation using API 50 CHL strips (bioMdrieux), following the manufacturer's instructions. The L. reuteri strain DSM 17938 was used as a positive control.

### Enzyme Profiling

The enzymatic profiles of L. reuteri strains were determined using the APIZym test strips (bioMdrieux), following manufacturer's instructions.

### Growth Kinetics

The L. reuteri strains were grown in MRS broth overnight for 14-16 hours under anaerobic conditions at 37° C. The next morning, the cultures were adjusted to an OD600 of 0.1 and monitored for growth by plating on Lactobacilli MRS agar at 0, 1, 2, 4 and 8 hours. Human L. reuteri strain 23272 and L. acidophilus strain ATCC 4356 were used as controls.

### Isolation of High Molecular Weight DNA

High molecular weight DNA for PacBio sequencing was isolated using the phenol:chloroform method. Briefly, L. reuteri strains were grown in Lactobacilli MRS broth overnight under anaerobic conditions for 14-16 hours without shaking. The cells were pelleted by centrifugation at 4,000 g RCF for 10 minutes at 4° C. The pellet was washed once in 1 mL of TE buffer (10 mM Tris-HCl and 1 mM EDTA, pH 8.0) and resuspended in 0.5 mL of TE containing 1.2% Triton X-100 and 10 mg/mL of lysozyme (Sigma Aldrich) and incubated at 37° C. for 1 hour. After incubation, 20 μL of proteinase K was added, mixed several times, and incubated at 55° C. for 1 hour. Twenty microliters of RNase was then added and incubated at 37° C. for an additional 30 minutes. Approximately, 600 μL of phenol:chloroform:isoamyl alcohol (25:24:1; ThermoFisher Scientific) mixture (pH 8.0) was added, the tubes were inverted several times and centrifuged at 10,000 rpm for 10 minutes at 4° C. The upper aqueous phase was carefully transferred to a new 1.5 mL centrifuge tube. The above phenol:chloroform step was repeated one more time. An equal volume of chloroform was added, the tubes were inverted several times and centrifuged at 10,000 rpm for 10 minutes. The upper aqueous phase was carefully transferred to a new centrifuge tube. An equal volume of isopropanol was layered onto the aqueous phase containing genomic DNA and the tubes were gently shaken to precipitate high molecular weight genomic DNA. The precipitated DNA was removed with a sterile loop and transferred to a new tube containing 1 mL 70% ethanol. The tubes were centrifuged at 10,000 rpm for 5 minutes at 4° C. The DNA pellet was briefly air-dried and 300 μL of nuclease free water was added, and then allowed to dissolve overnight at 4° C. The dissolved DNA was gently mixed with a big-bore tip and stored at −20° C. The isolated DNA was analyzed for quantity using Qubit.

### Whole Genome Sequencing and Assembly

The bacterial genomic DNA samples were shipped on dry-ice to DNA Link, Inc (San Diego, CA) for whole genome sequencing using PacBio RSII platform. Briefly, 20 kb DNA fragments were generated by shearing genomic DNA using the Covaris G-tube according to the manufacturer's recommended protocol (Covaris). Smaller fragments were purified by the AMpureXP bead purification system (Beckman Coulter). For library preparation, 5 μg of genomic DNA was used. The SMRTbell library was constructed using SMRTbell™ Template Prep Kit 1.0 (PacBio®). Small fragments were removed using the BluePippin Size selection system (Sage Science). The remaining DNA sample was used for large-insert library preparation. A sequencing primer was annealed to the SMRTbell template and DNA polymerase was bound to the complex using DNA/Polymerase Binding kit P6 (PacBio®). Following the polymerase binding reaction, the MagBead was bound to the library complex with MagBeads Kit (PacBio®). This polymerase-SMRTbell-adaptor complex was loaded into zero-mode waveguides. The SMRTbell library was sequenced by 2 PacBio® SMRT cells (PacBio®) using the DNA sequencing kit 4.0 with C4 chemistry (PacBio®). A 1×240-minute movie was captured for each SMRT cell using the PacBio® RS sequencing platform. The genome was further assembled by DNA link, Inc with HGAP.3 protocol.

### Genome Annotation and Feature Prediction

Genome annotation was carried out using a custom annotation pipeline by combining several prediction tools. Coding sequences, transfer RNA and transmembrane RNA were predicted and annotated using Prokka v 1.14.5 (36-38). Ribosomal binding site (RBS) prediction was carried out using RBSFinder (39). TranstermHP v2.08 was used to predict Rho-independent transcription terminators (TTS) (40). Ribosomal RNA and other functional RNAs such as riboswitches and non-coding RNA was annotated with Infernal v1.1.2 (41). Operons were predicted based on primary genome sequence information with Rockhopper v2.0.3 using default parameters (42).

### Data Deposition

The raw sequencing reads, genome assemblies and annotations in this study were deposited in the NCBI BioProject under project PRJNA675717.

Accession numbers:

| SerialNo | Sample | BioSample | Accession number | SRA number |
|---|---|---|---|---|
| 1. | ATCC PTA-126787 | SAMN16712075 | CP065330-CP065334 | SRX9689306 |
| 2. | ATCC PTA-126788 | SAMN16712076 | CP065849-CP065855 | SRX9689307 |

### Phylogenetic Analysis

Phylogenetic relationships of the genomes were explored with UBCG v3.0 using default settings (43). This software tool employs a set of 92 single-copy core genes commonly present in all bacterial genomes. These genes then were aligned and concatenated within UBCG using default parameters. The estimation of robustness of the nodes is done through the gene support index (GSI), defined as the number of individual gene trees, out of the total genes used, that present the same node. A maximum-likelihood phylogenetic tree was inferred using FastTree v.2.1.10 with the GTR+CAT model (44).

### Comparative Genomic Analysis

OrthoFinder v2.3.11(45) was used to determine orthologous relationships between protein sequences inferred from PTA-126787 and PTA-126788 with protein sequences of strains ATCC 53608, CF48-3A, DSM20016 and SD2112 (the parent strain of DSM17938) downloaded from GenBank (46). Pairwise Average Nucleotide Identities (ANI) values were calculated all-against-all, using FastANI v 1.32 (47).

### Identification of Prophages, Transposases and Other Insertion Sequences

Insertion sequence prediction was done using ISEscan v.1.7.2.1 (48). Prophage prediction was done using PhiSpy v4.2.6 which combines similarity- and composition-based strategies (49).

### Identification of CRISPR-Cas Sequences

Coding sequences for Clustered Regularly Interspaced Short Palindromic Repeats (CRISPR) and CRISPR-associated genes (Cas) were searched using CRISPRDetect version 2.2 (50). However, no CRISPR sequences were identified in both the genomes.

### Identification of Virulence Determinants and Antimicrobial Resistance Genes

Protein-encoding genes related to virulence were searched manually based on functional annotation of the genomes. Automated screening of whole genome sequences of both strains against the Virulence Factor Database (VFDB), a comprehensive repository of known bacterial virulence factors and other putative adverse metabolites (51), ARG-ANNOT (52), ResFinder (53) and NCBI-AMR databases (2020 Jun. 15) was performed using Abricate version 0.9.9 (54).

### Identification of Genes Encoding Toxic Metabolites

Analysis was performed on the genomes manually to identify homologs of histidine decarboxylase, tyrosine decarboxylase, lysine decarboxylase, ornithine decarboxylase, agmatine deiminase, agmatine::putrescine antiporter, multicopper oxidase and other potential genes involved in the production of biogenic amines.

### Genes Involved in Lactic Acid Production and Other Beneficial Metabolites

Sequences encoding putative genes involved in lactic acid production and other metabolites were identified by manual search of functional annotations.

### Antimicrobial Susceptibility Profiling

Antimicrobial susceptibility testing was performed using broth microdilution method, using LSB medium (Mueller Hinton broth containing 5% horse blood) following Clinical and Laboratory Standards Institute (CLSI, 28th edition) guidelines. Two-fold dilutions of the clinically relevant antibiotics (Clindamycin, Chloramphenicol, Erythromycin, Gentamicin, Kanamycin, Streptomycin, Tetracycline and Ampicillin, all purchased from Sigma Aldrich) were prepared in LSB medium. Approximately, 50 μL of 1×105 CFUs/mL of the L. reuteri cells were added into each well. “No antibiotic” and “medium” alone controls were included. Escherichia coli ATCC 25923, Pseudomonas aeruginosa ATCC 27853, Staphylococcus aureus ATCC 29213, Enterococcus faecalis ATCC 29212, Streptococcus pneumonia ATCC 49619 and Lacticaseibacillus paracasei ATCC 334 were used as quality control organisms. The plates were incubated for 24-48 hours under microaerophilic conditions. Minimum inhibitory concentration (MIC) was defined as the lowest concentration of antibiotic that showed complete inhibition of L. reuteri growth. The strains were classified as susceptible or resistant using the microbiological cut offs established by EFSA (35).

### Biogenic Amine Production

The ability of L. reuteri strains to produce biogenic amines was determined as previously described (55). Briefly, L. reuteri cultures were grown in MRS broth supplemented with L-tyrosine (0.1% m/v), L-histidine (0.1% m/v), L-arginine (0.1% m/v) or L-lysine (0.1% m/v) and pyridoxal-5-phosphate (0.005% m/v) under anaerobic conditions at 37° C. overnight. The cultures were then plated on supplemented decarboxylase broth base as described by Bover-Cid and Holzapfel (56) and colour development was recorded after 48 hours of incubation under anaerobic conditions at 37° C.

### D- and L-Lactate Production

The amounts of D- and L-lactate produced were quantified using D-/L-Lactic Acid (D-/L-Lactate) (Rapid) Assay Kit (Megazyme), following manufacturer's instructions. Briefly, L. reuteri strains were grown in MRS broth at 37° C. under anaerobic conditions for 14-16 hours. The cultures were adjusted to OD600 of 1.5 and centrifuged at 4,000 g RCF for 10 minutes at 4° C. The supernatant was filter sterilized and used for lactic acid quantification as—described in the manual provided by the manufacturer.

### Autoaggregation

The ability of L. reuteri strains to autoaggregate was assayed as follows. L. reuteri strains were grown in MRS broth overnight for 14-16 hours under anaerobic conditions at 37° C. without shaking. The cultures were adjusted to an OD600 of 0.1 and allowed to grow for another 14-16 hours and observed for aggregate formation. Autoaggregation was quantified as described previously with some minor modifications (57). L. reuteri strains were grown in MRS broth overnight for 14-16 hours under anaerobic conditions at 37° C. without shaking. The cultures were washed twice with PBS (pH 7.2) by centrifuging at 10,000 rpm for 10 minutes at 4° C. The washed cell pellets were then resuspended in PBS (pH 7.2) and adjusted to an OD600 of 0.5 to standardize the number of bacterial cells (107 to 108). The suspensions were incubated as 1-ml aliquots under anaerobic conditions at 37° C. for 5 hours. The OD600 was recorded after 5 hours. Autoaggregation percentage was calculated as follows: [1−(Absorbance at 5 hours/Absorbance at 0 hour)]×100.

### Hydrogen Peroxide Production

The ability of L. reuteri strains to produce hydrogen peroxide was assessed as previously described (58). Briefly, MRS agar plates were prepared with 0.25 mg/mL of tetramethylbenzidine and 0.01 mg/mL of horseradish peroxidase. L. reuteri strains were streaked on the supplemented MRS agar plates and incubated for 24 hours and coloration of the colonies/culture was recorded. White bacterial colonies/culture indicates no hydrogen peroxide production, pale blue colonies/culture indicates poor production and dark blue colonies/culture indicates high production.

### Resistance to Bile Salts

The ability of cultures to tolerate bile salts was determined as follows: L. reuteri strains were grown under anaerobic conditions for 14-16 hours at 37° C. without shaking. The culture was inoculated into fresh MRS broth (pH adjusted to 6.4) containing 0.3% bile salts (Oxoid, USA) at a rate of 1% inoculum and incubated under anaerobic conditions at 37° C. for 4 hours. Samples were collected at 0 hours and 4 hours after incubation and analysed for CFU counts.

### Resistance to Acidic pH

The tolerance of L. reuteri strains to low pH was determined as described below. L. reuteri strains were grown under anaerobic conditions for 14-16 hours at 37° C. without shaking. The cells were harvested by centrifugation at 4,000 g RCF for 10 minutes at 4° C. and the pellet was resuspended to an OD600 of 0.5 in sterile PBS adjusted to a pH of 2.5. The cultures were then incubated at 37° C. for 3 hours under anaerobic conditions. Aliquots of samples were collected at time 0 hours and 3 hours after incubation, serially diluted in PBS and plated on MRS to determine the CFU counts.

### In Vivo Safety Assessments

The safety of L. reuteri strains was tested using Specific Pathogen Free (SPF) chickens. Forty-two (Three groups, 14 chicks per group) day-old, White Leghorn, mixed sex chicks were purchased from Valo BioMedia. At arrival, the chicks were tagged via wing web. The birds were fed commercially available non-medicated feed ad libitum. Briefly, day-old chicks were randomly grouped into three groups with 14 birds in each group. Group 1 was administered with 1×107 CFUs/bird/day of L. reuteri PTA-126787 in drinking water from day 1 to day 26. Group 2 was administered with 1×107 CFUs/bird/day of L. reuteri PTA-126788 in drinking water from day 1 to day 26. Group 3 served as no treatment control. The birds were examined for adverse events, morbidity, and mortality on a daily basis. On day 31, the birds were euthanized, and observed for any gross lesions, indicative of health issues. More specifically, lungs, trachea, liver, spleen, kidneys, intestine were observed for gross lesions and scored as normal or abnormal. Gut (2 cm of cecal-rectal junction), lung (dime size) and tracheal (2 cm long) samples were collected from 5 birds per group in buffered formalin, analysed for histopathology and scored as described in TABLE 1.

TABLE 1
Histopathological Scoring Criteria

| | |
|---|---|
| TRACHEA | Cilia: 4 = complete deciliation, 3 = marked deciliation [75%], |
| | 2 = moderate deciliation [50-75%], 1 = patchy deciliation[<50%], |
| | 0 = NSF); |
| | Epithelium (4 = severe necrosis/sloughing or squamous |
| | metaplasia, 3 = marked necrosis with segmental |
| | sloughing/erosion, 2 = moderate necrosis, 1 = mild necrosis, |
| | 0 = NSF) |
| | Mucosa: 4 = marked/diffuse nodular lymphoid hyperplasia |
| | [4+/lpf] with marked hyperplasia of mucus glands OR complete |
| | destruction of mucus glands, 3 = moderate nodular lymphoid |
| | hyperplasia [3-4/lpf] and moderate hyperplasia of mucus |
| | glands, 2 = mild nodular lymphoid hyperplasia [1-2/lpf] with mild |
| | mucus gland hyperplasia, 1 = mild lymphoid infiltrates/no |
| | nodules, 0 = NSF) |
| | Inflammation: 4 = diffuse, marked heterophilic infiltration in |
| | lamina propria with margination of cells; 3 = focally extensive, |
| | moderate heterophilic infiltration in lamina propria with some |
| | margination, 2 = moderate (35-50%) heterophilic inflammation, |
| | 1 = mild (<35%) heterophilic infiltration, 0 = NSF) |
| LUNG | Parabronchi: 4 = diffuse hyperplastic parabronchitis with 5+ |
| | lymphoid nodular aggregates and heterophilic infiltrates and |
| | marked edema, 3 = focally extensive hyperplastic parabronchitis |
| | with 3-5 nodular aggregates and mild heterophilic infiltrates |
| | and edema, 2 = focal parabronchitis with <3 nodular aggreagtes |
| | and mild edema, 1 = mild lymphocytic infiltrates in lamina |
| | propria OR mild edema, 0 = NSF |
| CECAL | 4 = marked/diffuse nodular lymphoid hyperplasia [4+/lpf] with |
| TONSILS | marked hyperplasia of mucus glands, 3 = moderate nodular |
| | lymphoid hyperplasia [3-4/lpf] and moderate hyperplasia of |
| | mucus glands, 2 = mild nodular lymphoid hyperplasia [1-2/lpf] |
| | with mild mucus gland hyperplasia, 1 = mild lymphoid |
| | infiltrates/no nodules, 0 = NSF |

### Results

L. reuteri Isolation and Molecular Identification

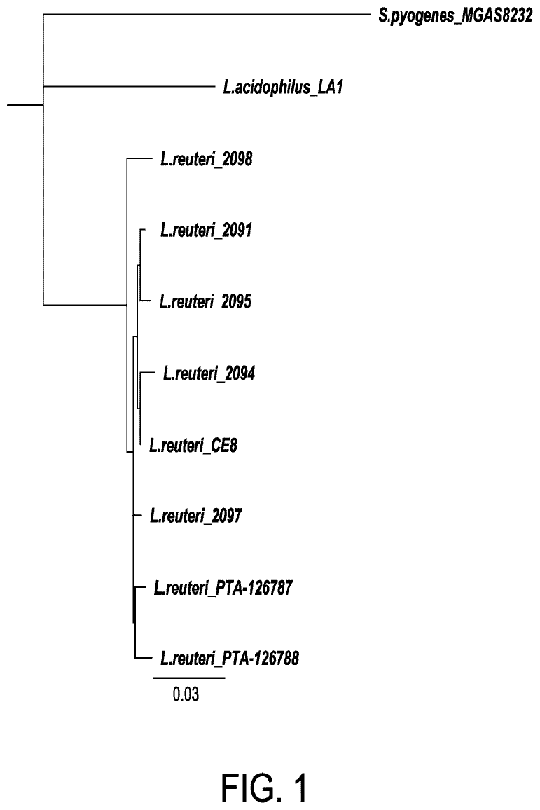

A library of seven L. reuteri strains along with the two strains described in this study were isolated from the cecum of older broiler chickens at Elanco Animal Health, Cuxhaven, (Germany). Based on the 16S rRNA amplicon sequencing and respective BLAST search comparison results, all the seven strains, including PTA-126787 and PTA-126788 showed closest homology to published L. reuteri sequences, suggesting that our strains belong to the L. reuteri species (FIG. 1).

### Biochemical Identification

When tested with API 50 CHL, the final two L. reuteri candidates, PTA-126787 and PTA-126788, were identified as Limosilactobacillus fermentum (previously Lactobacillus fermentum) with 92.3% identity. The positive control L. reuteri DSM 17938 was also identified as L. fermentum with 92.3% identity. The fermentation profile of L. reuteri is similar to that of L. fermentum and the APIweb™ software version 5.0 does not have the capability to distinguish between the 2 species. The carbohydrate fermentation profile of PTA-126787 and PTA-126788 are depicted in TABLE 2 and compared with the control DSM 17938 strain.

TABLE 2
Carbohydrate fermentation profile of L. reuteri
strains PTA-126787 and PTA-126788 by API 50 CHL

| | PTA- | PTA- | DSM | | PTA- | PTA- | DSM |
|---|---|---|---|---|---|---|---|
| Substrate | 126787 | 126788 | 17938 | Substrate | 126787 | 126788 | 17938 |
| Negative control | − | − | − | Esculin ferric | + | + | + |
| | | | | citrate | | | |
| Glycerol | − | − | − | Salicin | − | − | − |
| Erythritol | − | − | − | D-Cellobiose | − | − | − |
| D-Arabinose | − | − | − | D-Maltose | + | + | + |
| L-Arabinose | + | + | + | D-Lactose | + | + | + |
| D-Ribose | + | + | + | D-Melibiose | + | + | + |
| D-Xylose | − | − | − | D-Saccharose | + | + | + |
| L-Xylose | − | − | − | D-Trehalose | − | − | − |
| D-Adonitol | − | − | − | Inulin | − | − | − |
| Methyl-βD- | − | − | − | D-Melezitose | − | − | − |
| xylopyranoside | | | | | | | |
| D-Galactose | + | + | + | D-Raffinose | + | + | + |
| D-Glucose | + | + | + | Amidon | − | − | − |
| D-Fructose | − | − | − | Glycogen | − | − | − |
| D-Mannose | − | − | − | Xylitol | − | − | − |
| L-Sorbose | − | − | − | Gentibiose | − | − | − |
| L-Rhamnose | − | − | − | D-Turanose | − | − | − |
| Dulcitol | − | − | − | D-Lyxose | − | − | − |
| Inositol | − | − | − | D-Tagatose | − | − | − |
| D-Mannitol | − | − | − | D-Fucose | − | − | − |
| D-Sorbitol | − | − | − | L-Fucose | − | − | − |
| Methyl-αD- | − | − | − | D-Arabitol | − | − | − |
| mannopyroside | | | | | | | |
| Methyl-αD- | − | − | − | L-Arabitol | − | − | − |
| glucopyranoside | | | | | | | |
| N- | − | − | − | Potassium | + | + | + |
| Acetylglucosamine | | | | gluconate | | | |
| Amygdalin | − | − | − | Potassium 2- | − | − | − |
| | | | | ketogluconate | | | |
| Arbutin | − | − | − | Potassium 5- | − | − | − |
| | | | | ketogluconate | | | |

+, positive reaction;
−, negative reaction

### Enzyme Profile

Enzyme profile is a good indicator of both the probiotic function as well as safety. APIZym test is a apid semiquantitative assay to detect 19 enzymatic reactions. Unlike API 50 CHL, no databases exist to identify bacteria based on APIZym profiles. As shown in TABLE 3, both L. reuteri strains showed similar enzyme profiles. Both strains showed strong leucine arylamidase, valine arylamidase, acid phosphatase, α-galactosidase and β-galactosidase activities, while the two strains were negative for alkaline phosphatase, lipase, trypsin, α-chymotrypsin, β-glucosidase, α-mannosidase and α-fucosidase activities. In general, the enzymatic reactions from APIZym testing were in good agreement with carbohydrate fermentation by API 50 CHL.

TABLE 3
Enzymatic profiles of L. reuteri strains
PTA-126787 and PTA-126788 by APIZym.

| | | PTA- | PTA- |
|---|---|---|---|
| Enzyme assayed | Substrate | 126787 | 126788 |
| Alkaline phosphatase | 2-naphthyl phosphate | − | − |
| Esterase (C 4) | 2-naphthyl butyrate | + | + |
| Esterase Lipase (C 8) | 2-naphthyl capylate | +/− | +/− |
| Lipase (C 14) | 2-naphthyl myristate | − | − |
| Leucine arylamidase | L-leucyl-2-naphthylamide | +++ | +++ |
| Valine arylamidase | L-valyl-2-naphthylamide | ++ | ++ |
| Cystine arylamidase | L-cystyl-2-naphthylamide | + | + |
| Trypsin | N-benzoyl-DL-argine-2- | − | − |
| | naphthylamide | | |
| α-chymotrypsin | N-glutaryl-phenylalanine-2- | − | − |
| | naphthylamide | | |
| Acid phosphatase | 2-naphthyl phosphate | +++ | +++ |
| Naphthol-AS BI- | Naphthol-AS-BI-phosphate | + | + |
| phosphohydrolase α- | 6-Br-2-naphthyl-αD- | +++ | +++ |
| galactosidase | galactopyranoside | | |
| β-galactosidase | 2-naphthyl-βD- | +++ | +++ |
| | galactopyranoside | | |
| β-glucuronidase | Naphthol-AS-BI-βD- | + | + |
| | glucuronide | | |
| α-glucosidase | 2-naphthyl-αD- | + | + |
| | glucopyranoside | | |
| β-glucosidase | 6-Br-2-naphthyl-βD- | − | − |
| | glucopyranoside | | |
| N-acetyl-β- | 1-naphthyl-N-acetyl-βD- | +/− | +/− |
| | glucosaminide | | |
| glucosaminidase α- | 6-Br-2-naphthyl-αD- | − | − |
| mannosidase | mannopyranoside | | |
| α-fucosidase | 2-naphthyl-αL- | − | − |
| | fucopyranoside | | |

+, positive enzymatic reaction;
−, negative enzymatic reaction

### Growth Profiles

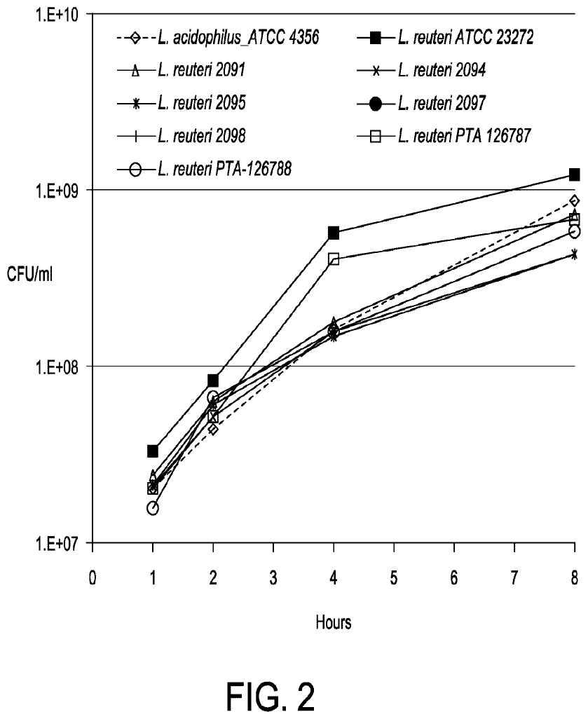

All the L. reuteri strains had similar growth profiles, including PTA-126787 and PTA-126788, and the profiles were comparable to that of human L. reuteri strain ATCC 23272 and Lactobacillus acidophilus strain ATCC 4356 (FIG. 2).

### In Silico Analyses

### A. Genomic Characterization

The genomes of L. reuteri strains PTA-126787 and PTA-126788 were sequenced by PacBio sequencing platform. Strain PTA-126787 (L. reuteri strain 3630) contains 5 contigs yielding a total estimated genome size of 2.4 Mb. The PTA-126787 (L. reuteri strain 3630) genome contigs nucleic acid sequences are provided in SEQ ID NO:44 through SEQ ID NO:48. Strain PTA-126788 (L. reuteri strain 3630) contains 7 contigs yielding an estimated genome size of 2.4 Mb. The PTA-126788 (L. reuteri strain 3632) genome contigs nucleic acid sequences are provided in SEQ ID NO:49 through SEQ ID NO:55. The genome properties, prediction and annotation of different features are summarized in TABLE 4. The whole-genome sequencing project was deposited at DDBJ/ENA/GenBank under BioProject number PRJNA675717. Accession numbers:
1. ATCC PTA-126787 SAMN16712075 CP065330-CP065334
2. ATCC PTA-126788 SAMN16712076 CP065849-CP065855

TABLE 4
Genomic properties of L. reuteri
strains PTA-126787 and PTA-126788.

| Feature | PTA-126787 | PTA-126788 |
|---|---|---|
| Contigs | 5 (SEQ ID | 7 (SEQ ID |
| | NO: 44-48) | NO: 49-55) |
| Coding sequence | 2427 | 2495 |
| Prophages | 6 | 8 |
| Mobile Element | 86 | 88 |
| Non-coding RNA | 21 | 17 |
| Operons | 501 | 541 |
| Ribosomal RNA | 18 | 17 |
| Ribosomal binding site | 2359 | 2396 |
| Transcription terminator | 1182 | 1241 |
| Riboswitch | 26 | 25 |
| Transfer RNA | 73 | 74 |
| Transfer-messenger RNA | 1 | 1 |

### B. Phylogenetic Analysis

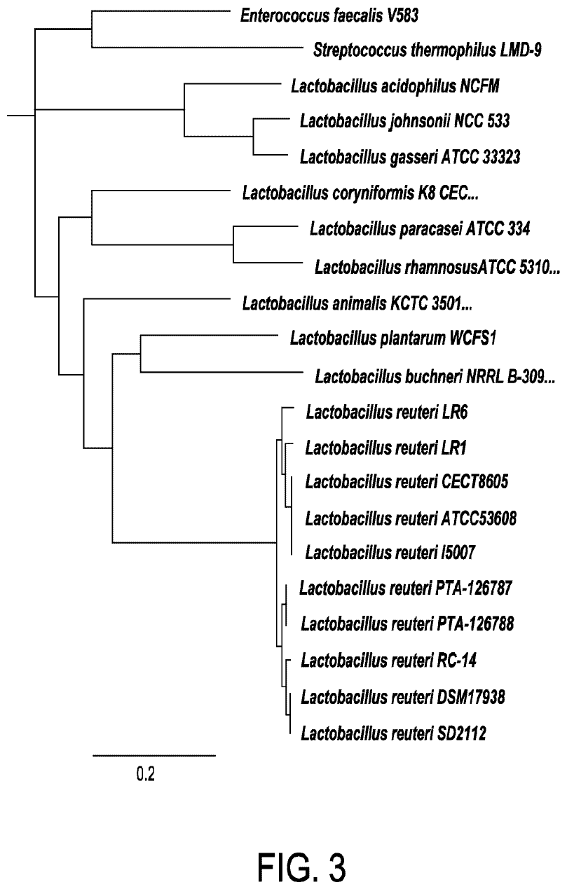

Phylogenetic relationships of the genomes were explored with UBCG v3.0 which employs a set of 92 single-copy core genes commonly present in all bacterial genomes. These genes then were aligned and concatenated within UBCG using default parameters. The estimation of robustness of the nodes is done through the gene support index (GSI), defined as the number of individual gene trees, out of the total genes used, that present the same node. As shown in FIG. 3, both strains PTA-126787 and PTA-126788 showed closest relationship to L. reuteri. Average Nucleotide Identities were calculated between closely related genomes and is shown in TABLE 5.

TABLE 5
Average Nucleotide Identity (ANI) of L. reuteri PTA-126787
and PTA-126788 with closely related human probiotic strains.

| Query | Reference | | Orthologous | Sequence |
|---|---|---|---|---|
| genome | genome | % ANI | matches | fragments |
| PTA-126787 | CF48-3A | 98.137 | 560 | 797 |
| PTA-126787 | RC-14 | 98.093 | 537 | 797 |
| PTA-126787 | RC-18 | 98.093 | 537 | 797 |
| PTA-126787 | SD2112 | 98.082 | 590 | 797 |
| PTA-126787 | DSM17938 | 98.080 | 591 | 797 |
| PTA-126787 | DSM20016 | 95.497 | 523 | 797 |
| PTA-126787 | ATCC53608 | 95.171 | 532 | 797 |
| PTA-126788 | CF48-3A | 98.221 | 553 | 825 |
| PTA-126788 | SD2112 | 98.149 | 581 | 825 |
| PTA-126788 | DSM17938 | 98.142 | 582 | 825 |
| PTA-126788 | RC-14 | 98.063 | 532 | 825 |
| PTA-126788 | RC-18 | 98.063 | 532 | 825 |
| PTA-126788 | DSM20016 | 95.609 | 516 | 825 |
| PTA-126788 | ATCC53608 | 95.028 | 545 | 825 |

### C. Comparative Genomics Analyses

Ortholog analysis was performed to identify paralogous and/or orthologous relationships between genomes of L. reuteri strains PTA-126787 and PTA-126788 against L. reuteri strains ATCC 53608, CF48-3A, DSM20016 and SD2112 (the parent strain of DSM17938) using OrthoFinder (TABLE 6 and 7). Genes unique to strains PTA-126787 and PTA-126787 are presented in TABLE 8. L. reuteri strains PTA-126787 and PTA-126788 shared the high number of orthologs amongst the strains compared in the analysis with 2264 and 2242 shared genes among them, respectively (TABLE 6).

TABLE 6
Orthologs shared between L. reuteri strains.

| | | | | PTA- | PTA- | |
|---|---|---|---|---|---|---|
| | ATCC53608 | CF48-3A | DSM20016 | 126787 | 126788 | SD2112 |
| ATCC53608 | 0 | 1597 | 1612 | 1572 | 1523 | 1612 |
| CF48-3A | 1568 | 0 | 1610 | 1680 | 1631 | 2082 |
| DSM20016 | 1583 | 1609 | 0 | 1621 | 1569 | 1614 |
| PTA- | 1582 | 1696 | 1628 | 0 | 2264 | 1715 |
| 126787 | | | | | | |
| PTA- | | | | | | |
| 126788 | 1553 | 1659 | 1589 | 2242 | 0 | 1677 |
| SD2112 | 1660 | 2148 | 1661 | 1788 | 1745 | 0 |

TABLE 7
Summary of ortholog statistics of L. reuteri strains.

| | ATCC | CF48- | DSM20 | PTA- | PTA- | |
|---|---|---|---|---|---|---|
| | 53608 | 3A | 016 | 126787 | 126788 | SD2112 |
| Number of genes | 1906 | 2164 | 1860 | 2397 | 2416 | 2300 |
| Number of genes in | 1764 | 2119 | 1769 | 2387 | 2303 | 2292 |
| orthogroups | | | | | | |
| Number of unassigned genes | 142 | 45 | 91 | 10 | 113 | 8 |
| Percentage of genes in | 92.5 | 97.9 | 95.1 | 99.6 | 95.3 | 99.7 |
| orthogroups | | | | | | |
| Percentage of unassigned genes | 7.5 | 2.1 | 4.9 | 0.4 | 4.7 | 0.3 |
| Number of orthogroups | 1652 | 2017 | 1706 | 2177 | 2123 | 2008 |
| containing species | | | | | | |
| Percentage of orthogroups | 63.8 | 77.8 | 65.8 | 84 | 81.9 | 77.5 |
| containing species | | | | | | |
| Number of species-specific | 5 | 8 | 0 | 13 | 7 | 0 |
| orthogroups | | | | | | |
| Number of genes in species- | 10 | 16 | 0 | 26 | 15 | 0 |
| specific orthogroups | | | | | | |
| Percentage of genes in species- | 0.5 | 0.7 | 0 | 1.1 | 0.6 | 0 |
| specific orthogroups | | | | | | |

TABLE 8
Genes unique to L. reuteri PTA-126787 and PTA-126788.

| Orthogroup | PTA-126787 | PTA-126788 | Annotation |
|---|---|---|---|
| OG0002869 | IU404_00186 | | hypothetical protein |
| OG0002870 | IU404_00494 | | Phosphate-binding protein PstS 1 |
| OG0002871 | IU404_00793 | | hypothetical protein |
| OG0002872 | IU404_01929 | | 30S ribosomal protein S1 |
| OG0002873 | IU404_02246 | | hypothetical protein |
| OG0002874 | IU404_02369 | | 50S ribosomal protein L19 |
| OG0002875 | IU404_02438 | | hypothetical protein |
| OG0002876 | IU404_02439 | | hypothetical protein |
| OG0002877 | IU404_02440 | | hypothetical protein |
| OG0002878 | IU404_02495 | | hypothetical protein |
| OG0002959 | | IVR12_02318 | hypothetical protein |
| OG0002879 | | IVR12_00335 | hypothetical protein |
| OG0002880 | | IVR12_00390 | hypothetical protein |
| OG0002881 | | IVR12_00406 | hypothetical protein |
| OG0002882 | | IVR12_00450 | Argininosuccinate synthase |
| OG0002883 | | IVR12_00667 | hypothetical protein |
| OG0002884 | | IVR12_00951 | hypothetical protein |
| OG0002885 | | IVR12_01333 | UDP-N-acetylmuramoyl-L-alanyl-D-glutamate-- |
| | | | L- lysine ligase |
| OG0002886 | | IVR12_01986 | hypothetical protein |
| OG0002887 | | IVR12_02184 | hypothetical protein |
| OG0002888 | | IVR12_02193 | hypothetical protein |
| OG0002889 | | IVR12_02204 | hypothetical protein |
| OG0002890 | | IVR12_02210 | UDP-N-acetylmuramate--L-alanine ligase |
| OG0002891 | | IVR12_02211 | Inner membrane protein YbhL |
| OG0002892 | | IVR12_02212 | hypothetical protein |
| OG0002893 | | IVR12_02213 | DNA polymerase I |
| OG0002894 | | IVR12_02214 | Formamidopyrimidine-DNA glycosylase |
| OG0002895 | | IVR12_02215 | Dephospho-CoA kinase |
| OG0002896 | | IVR12_02217 | Replication initiation and membrane attachment |
| | | | protein |
| OG0002897 | | IVR12_02218 | Primosomal protein DnaI |
| OG0002898 | | IVR12_02219 | hypothetical protein |
| OG0002899 | | IVR12_02220 | Translation initiation factor IF-3 |
| OG0002900 | | IVR12_02221 | 50S ribosomal protein L35 |
| OG0002901 | | IVR12_02222 | 50S ribosomal protein L20 |
| OG0002902 | | IVR12_02223 | hypothetical protein |
| OG0002903 | | IVR12_02224 | hypothetical protein |
| OG0002904 | | IVR12_02225 | hypothetical protein |
| OG0002905 | | IVR12_02228 | hypothetical protein |
| OG0002906 | | IVR12_02230 | putative protein YqeH |
| OG0002907 | | IVR12_02231 | RNA-binding protein |
| OG0002908 | | IVR12_02234 | Ribosomal silencing factor RsfS |
| OG0002909 | | IVR12_02235 | 2-methoxy-6-polyprenyl-1,4-benzoquinol |
| | | | methylase, mitochondrial |
| OG0002910 | | IVR12_02237 | hypothetical protein |
| OG0002911 | | IVR12_02238 | 50S ribosomal protein L32 |
| OG0002912 | | IVR12_02240 | Adaptive-response sensory-kinase SasA |
| OG0002913 | | IVR12_02242 | Inner membrane transporter YjeM |
| OG0002914 | | IVR12_02243 | Multidrug export protein MepA |
| OG0002915 | | IVR12_02245 | Membrane protein insertase MisCB |
| OG0002916 | | IVR12_02248 | 23S rRNA (guanosine-2′-O-)-methyltransferase |
| | | | RlmB |
| OG0002917 | | IVR12_02250 | HTH-type transcriptional regulator YodB |
| OG0002918 | | IVR12_02255 | Transcription elongation factor GreA |
| OG0002919 | | IVR12_02256 | hypothetical protein |
| OG0002920 | | IVR12_02258 | Penicillin-binding protein 2B |
| OG0002921 | | IVR12_02259 | Penicillin-binding protein 2B |
| OG0002922 | | IVR12_02260 | 50S ribosomal protein L33 1 |
| OG0002923 | | IVR12_02261 | putative protein YqgN |
| OG0002924 | | IVR12_02265 | Glucokinase |
| OG0002925 | | IVR12_02266 | putative protein YibN |
| OG0002926 | | IVR12_02267 | hypothetical protein |
| OG0002927 | | IVR12_02270 | Glutamate--tRNA ligase |
| OG0002928 | | IVR12_02271 | putative N-acetyl-LL-diaminopimelate |
| | | | aminotransferase |
| OG0002929 | | IVR12_02272 | IS30 family transposase ISHahy10 |
| OG0002930 | | IVR12_02273 | Aspartate/prephenate aminotransferase |
| OG0002931 | | IVR12_02274 | Aspartate aminotransferase |
| OG0002932 | | IVR12_02275 | putative N-acetyl-LL-diaminopimelate |
| | | | aminotransferase |
| OG0002933 | | IVR12_02276 | hypothetical protein |
| OG0002934 | | IVR12_02280 | Tyrosine recombinase XerC |
| OG0002935 | | IVR12_02282 | hypothetical protein |
| OG0002936 | | IVR12_02283 | hypothetical protein |
| OG0002937 | | IVR12_02284 | hypothetical protein |
| OG0002938 | | IVR12_02285 | hypothetical protein |
| OG0002939 | | IVR12_02286 | hypothetical protein |
| OG0002940 | | IVR12_02287 | hypothetical protein |
| OG0002941 | | IVR12_02288 | hypothetical protein |
| OG0002942 | | IVR12_02289 | hypothetical protein |
| OG0002943 | | IVR12_02290 | hypothetical protein |
| OG0002944 | | IVR12_02291 | hypothetical protein |
| OG0002945 | | IVR12_02294 | hypothetical protein |
| OG0002946 | | IVR12_02295 | hypothetical protein |
| OG0002947 | | IVR12_02296 | hypothetical protein |
| OG0002948 | | IVR12_02299 | hypothetical protein |
| OG0002949 | | IVR12_02300 | hypothetical protein |
| OG0002950 | | IVR12_02305 | hypothetical protein |
| OG0002951 | | IVR12_02307 | hypothetical protein |
| OG0002952 | | IVR12_02308 | hypothetical protein |
| OG0002953 | | IVR12_02310 | hypothetical protein |
| OG0002954 | | IVR12_02312 | hypothetical protein |
| OG0002955 | | IVR12_02313 | hypothetical protein |
| OG0002956 | | IVR12_02314 | hypothetical protein |
| OG0002957 | | IVR12_02316 | Endoribonuclease PemK |
| OG0002958 | | IVR12_02317 | Antitoxin MazE |
| OG0002959 | | IVR12_02318 | hypothetical protein |
| OG0002960 | | IVR12_02319 | hypothetical protein |
| OG0002961 | | IVR12_02320 | hypothetical protein |
| OG0002962 | | IVR12_02321 | hypothetical protein |
| OG0002963 | | IVR12_02322 | hypothetical protein |
| OG0002964 | | IVR12_02323 | hypothetical protein |
| OG0002965 | | IVR12_02324 | Chromosome partition protein Smc |
| OG0002966 | | IVR12_02325 | hypothetical protein |
| OG0002967 | | IVR12_02326 | hypothetical protein |
| OG0002968 | | IVR12_02327 | hypothetical protein |
| OG0002969 | | IVR12_02329 | Tyrosine recombinase XerC |
| OG0002970 | | IVR12_02330 | hypothetical protein |
| OG0002971 | | IVR12_02331 | hypothetical protein |
| OG0002972 | | IVR12_02333 | hypothetical protein |
| OG0002973 | | IVR12_02334 | hypothetical protein |
| OG0002974 | | IVR12_02335 | hypothetical protein |
| OG0002975 | | IVR12_02336 | hypothetical protein |
| OG0002976 | | IVR12_02337 | hypothetical protein |
| OG0002977 | | IVR12_02338 | Glutaredoxin-like protein NrdH |
| OG0002978 | | IVR12_02339 | hypothetical protein |
| OG0002979 | | IVR12_02340 | DNA topoisomerase 3 |
| OG0002980 | | IVR12_02346 | hypothetical protein |
| OG0002981 | | IVR12_02348 | hypothetical protein |
| OG0002982 | | IVR12_02350 | Signal peptidase IB |
| OG0002983 | | IVR12_02351 | hypothetical protein |
| OG0002984 | | IVR12_02352 | hypothetical protein |
| OG0002985 | | IVR12_02390 | hypothetical protein |
| OG0002986 | | IVR12_02440 | hypothetical protein |
| OG0002987 | | IVR12_02585 | hypothetical protein |
| OG0002988 | | IVR12_02601 | hypothetical protein |
| OG0002989 | | IVR12_02602 | hypothetical protein |
| OG0002990 | | IVR12_02603 | hypothetical protein |
| OG0002991 | | IVR12_02604 | hypothetical protein |

### D. Screening for Prophages, Insertion Sequences and Transposases

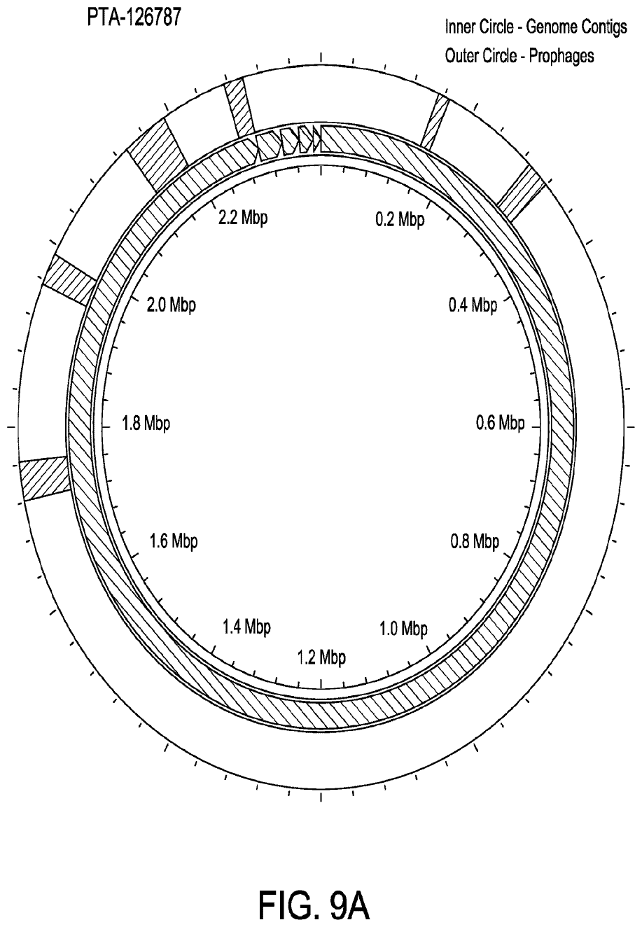

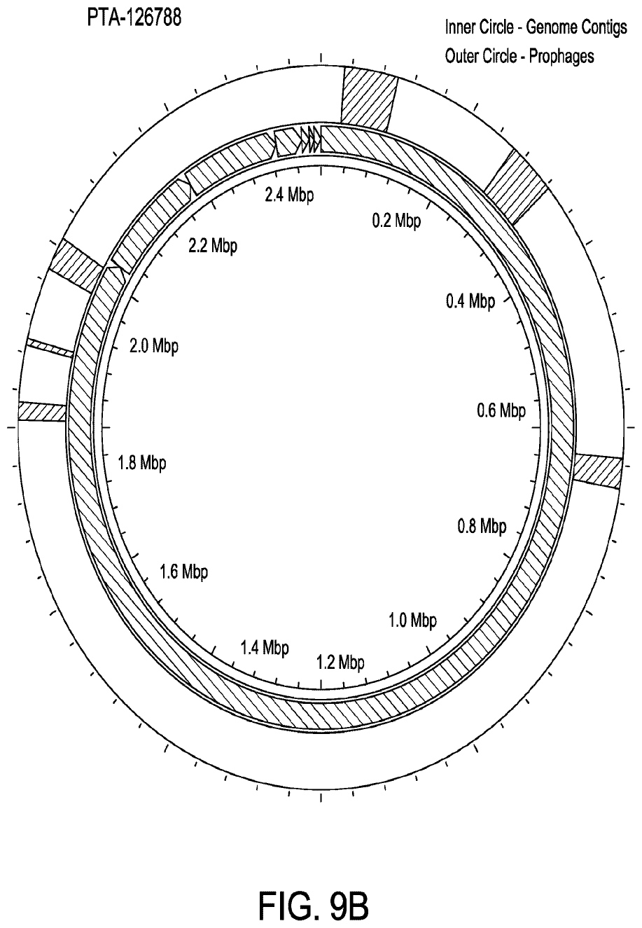

Both strains were scanned for the presence of mobile genetic elements such as prophages, insertion sequences (IS) and transposases. Six prophage regions in strain PTA-126787 and eight regions in PTA-126788 were identified (FIGS. 9 A and B; TABLE 9). However, there were 12 phage genes (all coding for Tyrosine recombinase protein) in PTA-126788 that were outside of prophage regions (TABLE 9). Putative IS and associated proteins predicted by ISEscan reveal 86 coding sequences in 10 IS families in strain PTA-126787 and 88 coding sequences in 18 IS families in strain PTA-126788 (TABLE 10; data not shown).

TABLE 9
Prophage in L. reuteri strains DSM
17938, PTA-126787 and PTA-126788.

| | | | |
|---|---|---|---|
| Chromosome | Feature | Start | End |

L. reuteri i DSM 17938

| | | | |
|---|---|---|---|
| DSM_1 | prophage_region | 162588 | 187315 |
| DSM_1 | attL | 161835 | 161847 |
| DSM_1 | attR | 187473 | 187485 |
| DSM_1 | prophage_region | 472700 | 511947 |
| DSM_1 | attL | 472715 | 472755 |
| DSM_1 | attR | 513707 | 513747 |
| DSM_1 | prophage_region | 527443 | 568241 |
| DSM_1 | attL | 527458 | 527498 |
| DSM_1 | attR | 568450 | 568490 |
| DSM_1 | prophage_region | 876952 | 907794 |
| DSM_1 | attL | 876967 | 877007 |
| DSM_1 | attR | 917959 | 917999 |
| DSM_1 | prophage_region | 2093038 | 2137900 |
| DSM_1 | attL | 2093053 | 2093093 |
| DSM_1 | attR | 2134045 | 2134085 |

L. reuteri i PTA-126788

| | | | |
|---|---|---|---|
| IU404_1 | prophage_region | 152457 | 167686 |
| IU404_1 | attL | 156168 | 156181 |
| IU404_1 | attR | 168138 | 168151 |
| IU404_1 | prophage_region | 290891 | 320979 |
| IU404_1 | attL | 289594 | 289607 |
| IU404_1 | attR | 319801 | 319814 |
| IU404_1 | prophage_region | 1720774 | 1762428 |
| IU404_1 | attL | 1718988 | 1719000 |
| IU404_1 | attR | 1763086 | 1763098 |
| IU404_1 | prophage_region | 1951294 | 1986058 |
| IU404_1 | attL | 1950720 | 1950733 |
| IU404_1 | attR | 1983717 | 1983730 |
| IU404_1 | prophage_region | 2129199 | 2187993 |
| IU404_1 | attL | 2132872 | 2132887 |
| IU404_1 | attR | 2184180 | 2184195 |
| IU404_1 | prophage_region | 2272336 | 2297075 |
| IU404_1 | attL | 2274787 | 2274924 |
| IU404_1 | attR | 2292319 | 2292456 |

L. reuteri i PTA-126788

| | | | |
|---|---|---|---|
| IVR12_1 | prophage_region | 59142 | 102453 |
| IVR12_1 | attL | 60071 | 60088 |
| IVR12_1 | attR | 102593 | 102610 |
| IVR12_1 | prophage_region | 272211 | 335768 |
| IVR12_1 | attL | 270691 | 270703 |
| IVR12_1 | attR | 333993 | 334005 |
| IVR12_1 | prophage_region | 653929 | 686757 |
| IVR12_1 | attL | 654858 | 654875 |
| IVR12_1 | attR | 697380 | 697397 |
| IVR12_1 | prophage_region | 1870956 | 1891239 |
| IVR12_1 | attL | 1870254 | 1870339 |
| IVR12_1 | attR | 1906483 | 1906568 |
| IVR12_1 | prophage_region | 1953921 | 1961107 |
| IVR12_1 | attL | 1953219 | 1953304 |
| IVR12_1 | attR | 1989448 | 1989533 |
| IVR12_1 | prophage_region | 2044154 | 2082229 |
| IVR12_1 | attL | 2043452 | 2043537 |
| IVR12_1 | attR | 2079681 | 2079766 |
| IVR12_2 | prophage_region | 57619 | 72552 |
| IVR12_2 | attL | 56917 | 57002 |
| IVR12_2 | attR | 93146 | 93231 |
| IVR12_3 | prophage_region | 31417 | 68097 |
| IVR12_3 | attL | 30715 | 30800 |
| IVR12_3 | attR | 66944 | 67029 |

TABLE 10
IS elements identified in L. reuteri
PTA-126787 and PTA-126788 genomes.
L. reuteri PTA-126787

| | | | % of | Length of | |
|---|---|---|---|---|---|
| | | | genome | sequence | |
| | | | sequence | segments | |
| | Family | | content | spanned | |
| | name | Number | spanned | by IS | Length of |
| Sequence | of IS | of IS | by IS | elements in | specific |
| identifier | element | copies | elements | a sequence | sequence |
| IU404_1 | IS110 | 6 | 0.41 | 9431 | 2297299 |
| IU404_1 | IS1182 | 3 | 0.23 | 5390 | 2297299 |
| IU404_1 | IS200/ | 19 | 1.35 | 31052 | 2297299 |
| | IS605 | | | | |
| IU404_1 | IS21 | 3 | 0.17 | 3921 | 2297299 |
| IU404_1 | IS3 | 5 | 0.35 | 7998 | 2297299 |
| IU404_1 | IS30 | 41 | 2.16 | 49546 | 2297299 |
| IU404_1 | IS66 | 5 | 0.42 | 9725 | 2297299 |
| IU404_1 | ISL3 | 1 | 0.04 | 806 | 2297299 |
| IU404_2 | IS30 | 2 | 44.04 | 16812 | 38177 |
| IU404_3 | IS30 | 1 | 4.56 | 1259 | 27631 |
| PTA- | total | 86 | 5.67 | 135940 | 2399045 |
| 126787 | | | | | |

L. reuteri PTA-126788

| | | | % | Length of | |
|---|---|---|---|---|---|
| | | | genome | sequence | |
| | | | sequence | segments | |
| | Family | | content | spanned | |
| | name | Number | spanned | by IS | Length of |
| Sequence | of IS | of IS | by IS | elements in | specific |
| identifier | element | copies | elements | a sequence | sequence |
| IVR12_1 | IS110 | 4 | 0.32 | 6649 | 2096256 |
| IVR12_1 | IS1182 | 2 | 0.17 | 3654 | 2096256 |
| IVR12_1 | IS200/ | 16 | 1.21 | 25318 | 2096256 |
| | IS605 | | | | |
| IVR12_1 | IS21 | 3 | 0.21 | 4430 | 2096256 |
| IVR12_1 | IS3 | 5 | 0.38 | 7998 | 2096256 |
| IVR12_1 | IS30 | 36 | 2.16 | 45270 | 2096256 |
| IVR12_1 | IS66 | 5 | 0.46 | 9725 | 2096256 |
| IVR12_1 | ISL3 | 1 | 0.04 | 806 | 2096256 |
| IVR12_2 | IS200/ | 4 | 3.76 | 6243 | 165920 |
| | IS605 | | | | |
| IVR12_2 | IS3 | 1 | 0.1 | 168 | 165920 |
| IVR12_2 | IS30 | 2 | 1.28 | 2118 | 165920 |
| IVR12_3 | IS110 | 2 | 1.98 | 2973 | 150023 |
| IVR12_3 | IS1182 | 1 | 1.19 | 1792 | 150023 |
| IVR12_3 | IS200/ | 2 | 2.14 | 3214 | 150023 |
| | IS605 | | | | |
| IVR12_3 | IS30 | 2 | 1.54 | 2316 | 150023 |
| IVR12_4 | IS200/ | 1 | 3.74 | 1583 | 42311 |
| | IS605 | | | | |
| IVR12_5 | IS200/ | 1 | 4.69 | 570 | 12147 |
| | IS605 | | | | |
| PTA- | total | 88 | 5.03 | 124827 | 2482713 |
| 126788 | | | | | |

### E. Absence of Virulence Factors and Toxins

Both L. reuteri PTA-126787 (5 contigs) and PTA-126788 (7 contigs) strains were confirmed to be free of known virulence factors and/or toxins by comparing against virulence factor database (VFDB; search parameters of >80% identity and >80% alignment length/coverage), which is an integrated comprehensive online resource database for curating information about bacterial virulence factors and/or toxins (51).

### F. Absence of Acquired Antimicrobial Resistance Genes

The Pariza et al. (34) decision tree and the EFSA Panel on Additives and Products or Substances used in Animal Feed (35) recommend that microbial strains used in food applications must not harbor acquired antimicrobial resistance genes to clinically relevant antimicrobials. Search for antimicrobial resistance genes was carried out for both L. reuteri strains by comparing the genomes against multiple AMR databases including NCBI-AMR, Resfinder DB and ARG-ANNOT using Abricate. The screening identified tetracycline-resistant ribosomal protection protein (tetW) that confers resistance to tetracycline as one potential gene of health concern (TABLE 11) (34). The tetW predicted protein sequence is shown below and the encoding nucleic acid sequence is further provided along with flanking sequences as well.

TABLE 11
Predicted antimicrobial resistance genes in
L. reuteri strains PTA-126787 and PTA-126788.

| | Chro- | | | Iden- | Cover- | Anno- |
|---|---|---|---|---|---|---|
| | mo- | | Size | tity | age | ta- |
| Strain | some | Location | (bp) | (%) | (%) | tion |
| PTA- | IU404_ | 723,894- | 1920 | 100 | 99.96 | Tet |
| 126787 | 1 | 725,813 | | | | (W) |
| PTA- | IVR12_ | 1,090,027- | 1920 | 100 | 99.96 | Tet |
| 126788 | 1 | 1,091,946 | | | | (W) |

tetW protein (SEQ ID NO: 58)
MKIINIGILAHVDAGKTTLTESLLYASGAISEPGSVKKGTTRTDTMF
LERQRGITIQAAVTSFQWHRCKVNIVDTPGHMDFLAEVYRSLAVLDG
AILVISAKDGVQAQTRILFHALRKMNIPTVIFINKIDQAGVDLQSVV
QSVRDKLSADIIIKQTVSLSPEIVLEENTDIEAWDAVIENNDKLLEK
YIAGEPISREKLVREEQRRVQDASLFPVYYGSAKKGLGIQPLMDAVT
GLFQPIGEQGSAALCGSVFKVEYTDCGQRRVYLRLYSGTLRLRDTVA
LAGREKLKITEMRIPSKGEIVRTDTAYPGEIVILPSDSVRLNDVLGD
PTRLPRKRWREDPLPMLRTSIAPKTAAQRERLLDALTQLADTDPLLR
CEVDSITHEIILSFLGRVQLEVVSALLSEKYKLETVVKEPTVIYKER
PLKAASHTIHIEVPPNPFWASIGLSVTPLPLGSGVQYESRVSLGYLN
QSFQNAVRDGIRYGLEQGLFGWNVTDCKICFEYGLYYSPVSTPADFR
SLAPIVLEQALKESGTQLLEPYLSFTLYAPREYLSRAYHDAPKYCAT
IETVQVKKDEVVFTGEIPARCIQAYRTDLAFYTNGQSVCLTELKGYQ
AADGKPVIQPRRPNSRLDKVRYMFQKIM

### G. Screening for Genes Involved in Biogenic Amines and Toxins

Functional annotation of the entire genomes of L. reuteri strains PTA-126787 and PTA-126788 revealed that they do not contain any known protein-encoding genes involved in the production of biogenic amines with the exception of CDS encoding for arginine deiminase. No other toxins were identified (TABLE 12).

TABLE 12
Identified protein-coding genes putative for arginine deiminase pathway.

| | | | | | |
|---|---|---|---|---|---|
| | | | | | Size |
| Locus tag | Gene function | Start | End | Strand | (bp) |

Strain 3630

| | | | | | |
|---|---|---|---|---|---|
| IU404_01433 | Ornithine carbamoyltransferase | 1,356,883 | 1,357,890 | Forward | 1008 |
| | (EC 2.1.3.3) | | | | |
| IU404_01450 | Arginine deiminase (EC | 1,372,477 | 1,373,709 | Forward | 1233 |
| | 3.5.3.6) | | | | |
| IU404_01452 | Arginine/ornithine antiporter | 1,374,302 | 1,375,723 | Forward | 1422 |
| | ArcD1 | | | | |
| IU404_01453 | Arginine/ornithine antiporter | 1,375,781 | 1,377,178 | Forward | 1398 |
| | ArcD1 | | | | |
| IU404_01451 | Arginine repressor | 1,373,820 | 1,374,281 | Forward | 462 |
| IU404_02399 | Arginine repressor | 2,199,921 | 2,200,373 | Reverse | 453 |

Strain 3632

| | | | | | |
|---|---|---|---|---|---|
| IVR12_01894 | Ornithine carbamoyltransferase | 1,720,823 | 1,721,830 | Forward | 1008 |
| | (EC 2.1.3.3) | | | | |
| IVR12_01911 | Arginine deiminase (EC | 1,736,417 | 1,737,649 | Forward | 1233 |
| | 3.5.3.6) | | | | |
| IVR12_00724 | Arginine/ornithine antiporter | 608,272 | 609,693 | Forward | 1422 |
| | ArcD1 | | | | |
| IVR12_01913 | Arginine/ornithine antiporter | 1,738,242 | 1,739,663 | Forward | 1422 |
| | ArcD1 | | | | |
| IVR12_01914 | Arginine/ornithine antiporter | 1,739,721 | 1,741,118 | Forward | 1398 |
| | ArcD1 | | | | |
| IVR12_01912 | Arginine repressor | 1,737,760 | 1,738,221 | Forward | 462 |

### H. Genes Involved in the Production of Lactic Acid and Other Beneficial Metabolites

Both strains, PTA-126787 and PTA-126788, contain genes responsible for production of lactic acids. A total of four coding sequences (CDS) were predicted to encode for D-lactate dehydrogenase (EC 1.1.1.28) and four CDS for L-lactate dehydrogenase (EC 1.1.1.27) were found on different loci within the genome (TABLE 13). However, IVR12_00498 gene in strain PTA-126788 is a pseudogene due to a frameshift mutation. The coding sequence putative for a therapeutically useful peptide, S-ribosylhomocysteinelyase (EC 4.4.1.21; IU404_00512 and IVR12_00964) was also present in the genomes of strains PTA-126787 and PTA-126788, respectively.

Several coding sequences involved in adhesion of Lactobacilli to intestinal epithelium were identified in the genome (TABLE 14). Some of the genes involved in adhesion to host found in both strains are sortase A, epsilon subunit related 3′-5′ exonuclease, exopolysaccharide biosynthesis protein and ATP synthase epsilon subunit (TABLE 14). Search for desired stress tolerance features in both L. reuteri strains revealed the presence of CDS predictably encoding for DNA protection during starvation protein (TABLE 14). Another stress resistant gene putatively encoding for Phosphate starvation-inducible PhoH-like protein, predicted ATPase was also found in both strains (TABLE 14).

TABLE 13
Putative genes in PTA-126787 and PTA-126788 involved in lactic acids production.

| | | | | | Size | |
|---|---|---|---|---|---|---|
| Gene function | Strain | Chromosome | Position | Locus Tag | (bp) | Strand |
| D-lactate | PTA- | IU404_1 | 531,116- | IU404_00583 | 1,014 | Reverse |
| dehydrogenase (EC | 126787 | | 532,129 | | | |
| 1.1.1.28) | | IU404_1 | 606,464- | IU404_00662 | 1,086 | Forward |
| | | | 607,549 | | | |
| | | IU404_1 | 866,523- | IU404_00944 | 996 | Reverse |
| | | | 867,518 | | | |
| | | IU404_1 | 1,632,717- | IU404_01740 | 993 | Forward |
| | | | 1,633,709 | | | |
| | PTA- | IVR12_1 | 409,989- | IVR12_00498 | 992 | Reverse |
| | 126788 | | 410,980 | | | |
| | | IVR12_1 | 897,268- | IVR12_01036 | 1014 | Reverse |
| | | | 898,281 | | | |
| | | IVR12_1 | 972,605- | IVR12_01115 | 1086 | Forward |
| | | | 973,690 | | | |
| | | IVR12_1 | 1,230,501- | IVR12_01396 | 996 | Reverse |
| | | | 1,231,496 | | | |
| L-lactate | PTA- | IU404_1 | 72,380- | IU404_00084 | 939 | Forward |
| dehydrogenase (EC | 126787 | | 73,318 | | | |
| 1.1.1.27) | | IU404_1 | 386,913- | IU404_00448 | 942 | Reverse |
| | | | 387,854 | | | |
| | | IU404_1 | 1,664,098- | IU404_01769 | 960 | Reverse |
| | | | 1,665,057 | | | |
| | | IU404_1 | 1,871,024- | IU404_02013 | 975 | Forward |
| | | | 1,871,998 | | | |
| | PTA- | IVR12_1 | 171,961- | IVR12_00207 | 975 | Reverse |
| | 126788 | | 172,935 | | | |
| | | IVR12_1 | 378,643- | IVR12_00467 | 960 | Forward |
| | | | 379,602 | | | |
| | | IVR12_1 | 752,015- | IVR12_00899 | 942 | Forward |
| | | | 752,956 | | | |
| | | IVR12_1 | 1,968,618- | IVR12_02186 | 939 | Reverse |
| | | | 1,969,556 | | | |

Note:
IVR12_00498 encoding D-lactate dehydrogenase (EC 1.1.1.28) is a pseudogene due to frameshift.

TABLE 14
Identified protein-encoding genes putative for adhesion
by L. reuteri strains PTA-126787 and PTA-126788.

| | | | |
|---|---|---|---|
| Locus Tag | Size | Gene Function | Strand |

Strain PTA-126787

| | | | |
|---|---|---|---|
| IU404_01324 | 178 | exonuclease | Reverse |
| IU404_02160 | 82 | exopolysaccharide biosynthesis protein | Reverse |
| IU404_01408 | 143 | F0F1 ATP synthase subunit epsilon | Forward |
| IU404_01134 | 234 | class A sortase | Forward |

Strain PTA-126788

| | | | |
|---|---|---|---|
| IVR12_01729 | 178 | exonuclease | Reverse |
| IVR12_02549 | 82 | exopolysaccharide biosynthesis protein | Forward |
| IVR12_01813 | 143 | F0F1 ATP synthase subunit epsilon | Forward |
| IVR12_01541 | 234 | class A sortase | Forward |

### Antimicrobial Susceptibility

Minimum inhibitory concentrations were analyzed against relevant antibiotics according to EFSA guidelines (EFSA Panel on Additives and Products or Substances used in Animal Feed) (35), including Ampicillin, Vancomycin, Gentamicin, Kanamycin, Streptomycin, Erythromycin, Clindamycin, Tetracycline and Chloramphenicol. L. reuteri PTA-126788 and PTA-126787 strains were determined to be sensitive to all relevant tested antibiotics according to EFSA guidelines (35), with MIC values at or below the reported species characteristic cut-off values (TABLE 15), except for tetracycline. For tetracycline, the MIC values for our strains were two-fold dilution above the EFSA microbiological cut off value, in one of the two biological replicates. However, this is considered acceptable due to the technical variation of the phenotypic method as recognized previously (59).

TABLE 15
Susceptibility of L. reuteri PTA-126787 and PTA-
126788 to EFSA Critically Important Antibiotics.

| | | | EFSA microbiological |
|---|---|---|---|
| | L. reuteri | L. reuteri | cut off values for |
| | PTA-126788 | PTA-126787 | L. reuteri |
| Clindamycin | ≥0.06 | ≥0.06 | 4 |
| Chloramphenicol | 2 | 2 | 4 |
| Erythromycin | 0.12 | 0.12 | 1 |
| Gentamicin | 1 | 1 | 8 |
| Kanamycin | 16 | 16 | 64 |
| Streptomycin | 8 | 8 | 64 |
| Tetracycline | 32/64 | 32/64 | 32 |
| Ampicillin | 1 | 1 | 2 |

### Biogenic Amine Production

Many lactic acid bacteria produce biogenic amines such as histamine, tyramine, putrescine and/or cadaverine by amino acid decarboxylation of histidine, tyrosine, ornithine and/or lysine, respectively. The few instances of toxicity cases are associated with histamine and to some extent tyramine. Consistent with the bioinformatics results, neither of the subject L. reuteri strains were able to produce the major biogenic amines histamine, tyramine, putrescine or cadaverine. As expected, L. reuteri ATCC 23272 produced a positive reaction in the area of bacterial growth on the decarboxylase base media supplemented with L-histidine. Control plates lacking these amino acids showed no positive reaction for any of the strains tested.

### D- and L-Lactate Production

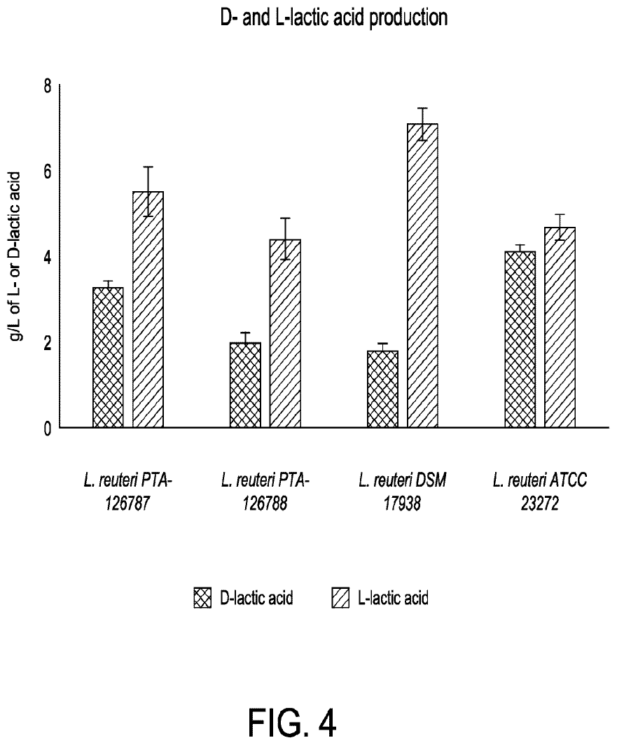

Quantitative determination of lactic acid production showed that the two L. reuteri strains produce both L- and D-lactic acids but predominantly L-lactic acid (FIG. 4). Similarly, L. reuteri DSM 17938 also produced L- and D-lactic acids but predominantly L-lactic acid (FIG. 4). L. reuteri strain ATCC 23272 produced approximately equal amounts of L- and D-lactic acids (FIG. 4).

### Autoaggregation

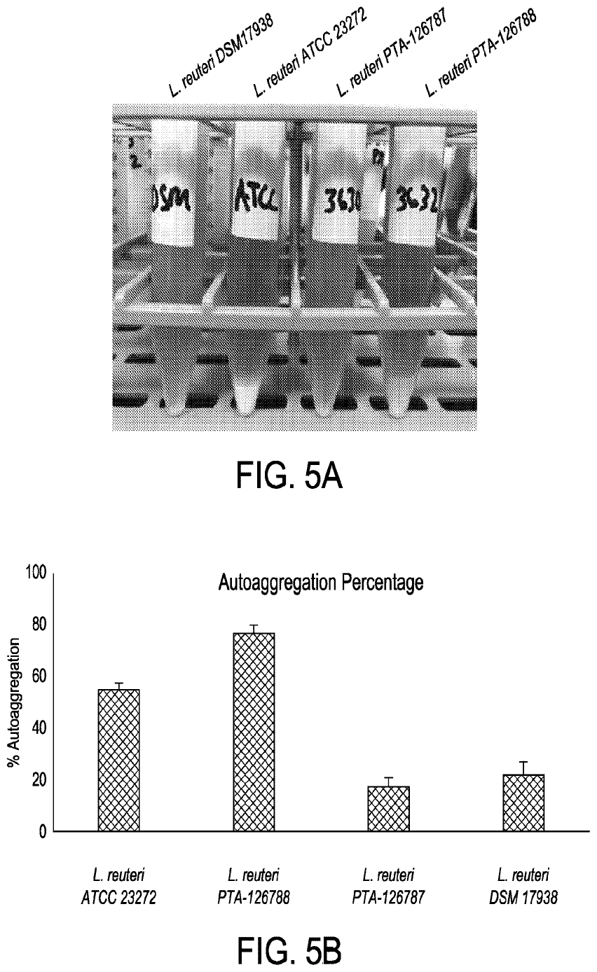

Autoaggregation is a phenomenon where bacteria form fibrous-like aggregates after overnight growth and settle to the bottom of the tube. Once the bacteria are aggregated, they generally do not redisperse unless vigorously mixed manually. Autoaggregation appears to be one of the key properties needed for probiotic strains to attach to the epithelial cells in the gastrointestinal tract. The ability to aggregate has also been suggested to play a role in preventing pathogen colonization. As shown in FIG. 5A, L. reuteri PTA-126788 showed excellent ability to autoaggregate, while the other L. reuteri strains PTA-126787 and DSM 17938 showed no ability to form aggregates. Similar to L. reuteri PTA-126788, the positive control, L. reuteri ATCC 23272 also showed ability to autoaggregate (FIG. 5A). Quantification of autoaggregation showed that L. reuteri PTA-126788 (strain 3632) exhibited the highest ability to autoaggregate, while L. reuteri strains PTA-126787 and DSM 17938 had the least ability to form aggregates (FIG. 5B). The positive control L. reuteri ATCC 23272 showed moderate ability to autoaggregate (FIG. 5B).

### Hydrogen Peroxide Production

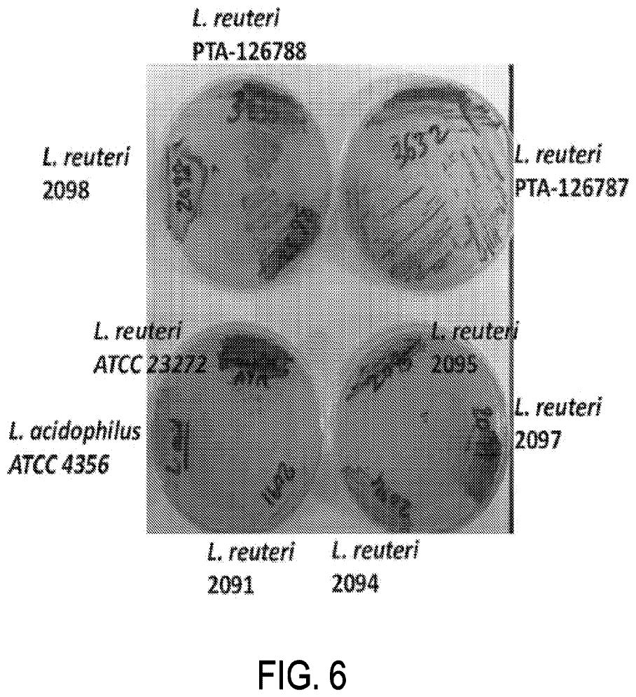

The ability of probiotic strains to produce hydrogen peroxide at physiological levels is highly desirable. Hydrogen peroxide production by Lactobacillus johnsonii has been attributed to inducing recovery of the epithelial barrier and remission in inflammatory bowel disease (60). Similarly, an L. reuteri probiotic strain producing hydrogen peroxide was able to significantly reduce proinflammatory response and improved clinical outcomes in human patients with chronic periodontitis (61). All the L. reuteri strains including PTA-126787 and PTA-126788 strains showed moderate to high ability to produce hydrogen peroxide as shown in FIG. 6.

### Resistance to Bile

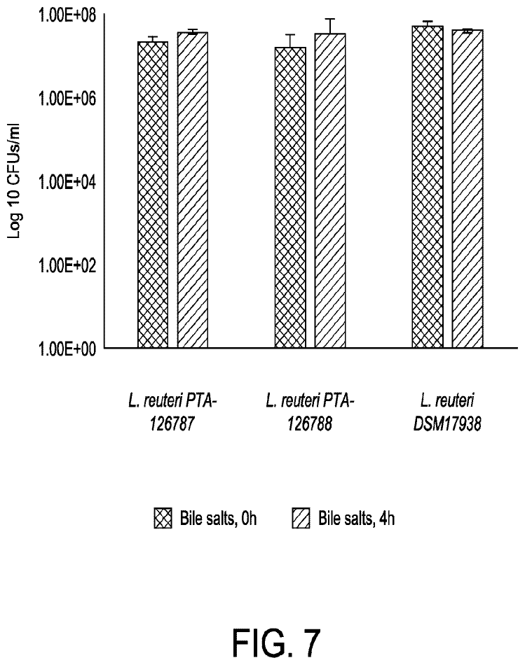

The ability of the two L. reuteri strains to tolerate bile salts was also assessed by incubating the strains in the presence of 0.3% bile salts. The viability of L. reuteri PTA-126787, PTA-126788 and DSM 17938 did not change after incubation with 0.3% bile salts for 4 hours, suggesting that our strains are resistant to 0.3% bile salts similar to DSM 17938 (FIG. 7).

### Resistance to Acidic pH

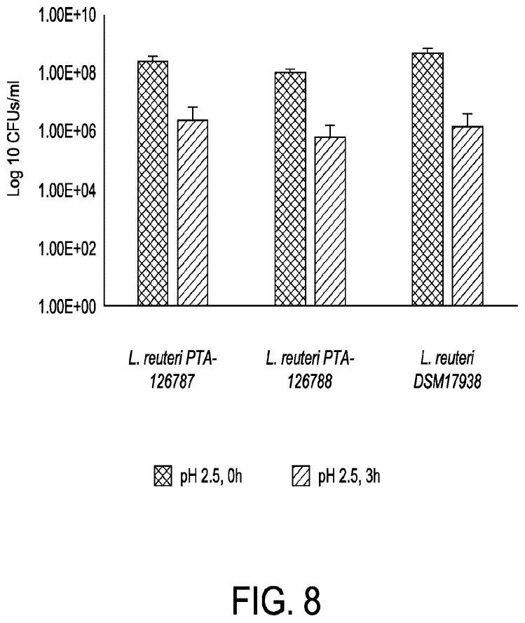

The viability of L. reuteri PTA-126787 decreased from 2.81×108 to 2.75×106 after incubation at pH 2.5 for 3 hours (FIG. 8). Similarly, the viability of L. reuteri PTA-126788 decreased from 1.22×108 to 6.67×105 after 3 hours incubation at pH 2.5 (FIG. 8). Overall, there was approximately 2-log reduction in viability for PTA-126787 and approximately 2.5-log reduction in viability for PTA-126788 after incubation at pH 2.5 for 3 hours (FIG. 8). The control strain DSM 17938 also showed approximately 2.5-log reduction in CFU counts from 5.51×108 to 1.57×106 after 3 hours incubation at pH 2.5 (FIG. 8).

### In Vivo Safety in Broilers

Compared to untreated control, groups treated with L. reuteri PTA-126787 and PTA-126788 daily in drinking water had no mortality, morbidity or adverse events in broiler chickens. Necropsy on day 26 showed no gross lesions indicative of health issues. Compared to the control group, histopathological analysis of trachea, lung and cecal tonsils from the groups treated with L. reuteri PTA-126787 or PTA-126788 showed no evidence of inflammation or abnormal pathology compared to untreated group (data not shown).

### Discussion

Our understanding of the microbiome and its impact on human and animal health is rapidly evolving, leading to the identification of innovative ways to impact human and animal diseases. Undoubtedly, the past two decades have witnessed a tremendous progress in the area of probiotics highlighting their key role in supporting general health, enhancing immune function and showing the potential to reduce specific diseases. Research has repeatedly shown that the survival, safety, and efficacy properties of probiotic candidates are strain specific and cannot be generalized. In the present study, we isolated two novel Lactobacillus strains from chicken, identified them as L. reuteri and established their safety using various genomic, in vitro, and in vivo studies, supporting their application as potential probiotics for human and animal health.

Identification is the first step in establishing the safety of a probiotic candidate and regulatory agencies recommend that at least two state-of-the-art methods be used to correctly identify a probiotic candidate (35, 62). API 50 CHL analysis identified our strains as L. fermentum. L. reuteri is a subtype of L. fermentum and the two species are indistinguishable at the biochemical level (63).

16S rRNA identification confirmed that our strains have closest homology to L. reuteri strains. Whole-genome sequencing coupled with phylogenetic analyses further confirmed that our strains have closest relatedness to L. reuteri and that our strains genetically cluster with DSM 17938, SD2112 (parent strain of DSM 17938) and RC-14. DSM 17938 and RC-14 are widely used as part of several commercially marketed dietary supplements and functional foods and there exists a plethora of clinical evidence supporting their safety and efficacy for different disease indications in humans. Consistent with our findings, several previous whole-genome phylogenetic studies also showed that the parent strain of DSM 17938, SD2112 indeed clusters with poultry isolates under poultry/human lineage IV and the authors from these studies hypothesized that SD2112 may have indeed originated from poultry (64-67). All together, these findings clearly establish that our strains belong to L. reuteri species and that our strains have closest homology to the two commercially marketed probiotic candidates with proven human clinical safety, DSM 17938 and RC-14.

Long read sequencing technology enabled complete genome characterization with each chromosome and plasmid represented by large, nearly complete contigs. Comprehensive functional annotation of the L. reuteri strains PTA-126787 and PTA-126788 revealed presence of several genes important for probiotic efficacy. Probiotic bacteria are known to contain bioactive secondary metabolites that interact with other pathogenic bacteria to attenuate virulence (68-71). For instance, lactic acids produced by lactic acid bacteria inhibit the growth and survival of nearby pathogens and inactivate human immunodeficiency virus by increasing acidity of the surrounding environment (72). Both PTA-126787 and PTA-126788 strains contain four coding sequences encoding D-lactate dehydrogenase (EC 1.1.1.28) and four encoding L-lactate dehydrogenase (EC 1.1.1.27) which are responsible for lactic acid production. However, IVR12_00498 from strain PTA-126787 is a pseudogene due to a frameshift mutation.

One of the key desirable traits in a probiotic candidate is the ability to adhere to epithelial cells. The genes identified in both strains of L. reuteri putatively encode proteins involved in adhesion, providing stability to the strains and the ability to compete with other undesirable resident gut bacteria, thereby enabling effective colonization of the gut and exclusion of pathogens (73, 74). Sortase-dependent proteins are an important group of cell surface proteins in Lactobacillus spp. and are responsible for sorting various kinds of cell surface proteins, thus playing an important role in adhesion (75). The genomes of both strains contain the gene encoding phosphate starvation-inducible protein PhoH, a member of both the Pho regulon and the sB-regulated general stress regulon. Pho regulon plays a key role in regulating phosphate homeostasis and is generally induced in response to phosphate starvation. The sB-dependent general stress proteins are predicted to provide cells with several kinds of non-specific stress tolerance (76).

While the diversity of phages in gut ecosystems is getting increasingly well-characterized, knowledge is limited on how phages contribute to the evolution and ecology of their host bacteria (77, 78). Prophage analysis of L. reuteri strain DSM 17938 showed 5 prophage regions while the strains, PTA-126787 and PTA-126788 had 6 and 8 prophage regions, respectively (S9 Table). Prophages can be advantageous for gut symbionts like L. reuteri by increasing its competitiveness in the intestinal niche (77).

Genome analysis identified the presence of tetW in both L. reuteri strains. tetW was found to be present on the chromosome and no elements indicative of horizontal transfer (plasmids, phages, transposons, or conjugation elements) were identified in the 15-kb flanking regions on both sides of tetW. The nucleic acid sequences encoding tetW and the flanking sequences for each of the PTA-126787 and PTA-126788 are shown below. Alignment of the tetW of the two strains and along with other neighboring genes was conducted and the tetW and flanking regions for PTA-126787 and PTA-126788 are 100% identical. Phenotypic analysis showed that the two strains are susceptible to all clinically relevant antimicrobials with MICs below the EFSA recommended microbiological cut offs, except for tetracycline. For tetracycline, both strains showed a marginal 2-fold increase in MIC than the recommended microbiological cut off and a 2-fold variation in the MIC is considered acceptable due to technical variation in the MIC assay and hence the strains can be considered phenotypically susceptible (59, 79). Together, these data suggest that the presence of tetW in our L. reuteri poses minimal risk to human and animal health.

>L. reuteri PTA-126787 (Strain 3630) tetW sequence along
with flanking gene sequence stdh CDS
(SEQ ID NO: 59)
TTAAGCTTCTTCGCCGCCAACCTTAACAACAGCTTTTGAAACCTTACCTAATTTACCACTAACAA
AGTCTTCAACACCATCAAGGTCTAATTCGTGTGAGAAGAGTGGTAGTGGGTCAACAACACCAGAA
GAAAGAAGTGCAATTGAGTCTTCAAAAGTGTAAGGGTTAATGAAGGCCCCTTGAATTGTAAGTTG
TTTTTGGAATACATCGTAAGTGTTTACTGAGAACTTGTCATCAGGGTTACCAACACCAAACATTA
ATACTTGAGCACCACGAGCTGCAGCTGCAAGTGCTTGTTCTTGAGTAGCTGGCAAACCAACCGCT
TCAACAACGATGTCATATGCATCAGCAGGAATTTCTTCCTTAGTAGTGTTGATAGTCTTAACGCC
GAAGTGCTTCCGGTTGTTTTCCAACTTTTCATCTGAACGACCAGCTAAAGTAACTTCGTGAATAC
CACGAGCCTTCAAGATTTGTGCAAATAATTGACCTTCAAAACCATCACCTAATACTAAAGCCTTT
TGGTATGGGTGAGTTTCAAGTAAGTCAACACCGTGCATTGCACATGAAATTGGTTCAACAACGGC
AGCTGCCTTTAATGAAACATCATCAGGAATTGGGTAAACAACTTTTGCAGGAGCAGTAAAGTATT
CTTCAAAACCACCGTTACGGGTAACACCAACAGCATCAAGGTGTTCACATAATTCTGGGCGTTGA
GTCCGACAATACTTACATTGACCACAGTAGATGTTAGGGTCAACGGTTACACGATCACCTGGCTT
AACGTTAGTAACTTCTGAACCAACTTTTGTAACTACCCCTGAGTTTTCATGACCTAAAACGATAG
GAGGAACAGCTGATGCAGATCCCGGAAGACCGGCATAGAGCGCTTTATCAGTACCACAAATACCA
GCGTAGGCAGTATGAATTAAGACTTCATCAGGCTTAATTTCAGGTTCTTTAATATCTTCAATTTC
GAGTTGTTTTTTACCTGTTAAAACAAGTGCCTTCAT
Intervening sequence
(SEQ ID NO: 60)
AATTAAAATACCTCTCTTGTGAATTTTATCATTTGTCAAGTGATAGTATAGTGTGAAAACGCCTT
AATTGTCAAACGTCCAACAATTATCTCACATTCTTTTCGCCATCATAAAACGTTAAACTCAGCAA
ATCCGAACCTAAATAAATCCCGGTATAGAAGCATTTTCTAAAAAAGTGCAATTTTTAAATATTTT
TTTCTTCACTAAGTTTATGCTATTTTTTTGTTATCACATAAATGGTTTTACTTTCATTTTTTAAA
TTACATAATTTCACAAGGCTTTTTATCCTATTTAGCGTTTTACATCAAGAAATAAAATGTTTCTA
CATATAAAGTAATAAATTCTAAATTTATATAGAAAATTTCTCCAACTTTCAGAAAGCGCTTAACT
ACCATTAGGCACGCCAAAACTTATCTCCAACCGCTCCTCAAAATCGCCCAAATGGCCGTTATAAC
GGCTATTTTCCATTGCAGGATATATTTTTTTATGTTACCCTAACAAAGTATTCGAACTTAGTTAA
TTATGATTTCCAATCTAATTAACAAAACTCAAACGAAAGGTGGATTTGGC
mntH CDS
(SEQ ID NO: 61)
GTGGACGACACAAAGAATCAACACCGGAAGCATAAACTTATTGAATATGCTAACGGTAAATCACT
AGAGGAAATCAACGGAACAGTTGAAGTTCCTCGTGGAAAAGGCTTCTGGCGAACATTATTCGCTT
ACTCTGGTCCCGGTGCATTAGTTGCTGTGGGTTACATGGATCCAGGTAACTGGTCAACTTCAATT
ACTGGTGGACAGAGTTTCCAATATACCTTAATGACTACTATCTTGATTTCAAGTTTGATTGCGAT
GTTGCTTCAATACATGGCGGCTAAACTCGGAATCGTGAGCCAAATGGACCTTGCTCAGGCAACAC
GGGCACGTACCGGTAAAGCATTAGGTATTATCTTATGGATTATGACTGAGTTGGCGATTATGGCT
ACTGATATCGCTGAAGTTATCGGGGCTGCTATTGCTTTAAACTTACTGTTCCACATTCCGTTGAT
CCCATCTGTATTTATTACTGTTCTTGATGTTTTAGTACTGCTATTATTAACCAAGATCGGATTCC
GGAAGATTGAAGCAATTGTTGCATGTTTGATCTTGGTAATCTTGTTTGTTTTTGCTTACCAAGTT
GCCTTATCTAACCCTAACTGGGGTGGCGTATTTATGGGTCTCCTTCCATCAGCTAAGGCAATTGC
CCAACATCCAGAAATTAGTGGTATTACTCCATTAACTGGATCATTAGGTATTATCGGTGCGACTG
TTATGCCTCACAACCTATATCTCCACTCAGCAATTTCTCAAACTCGGAAGATTGACCACAATGAT
CTTGATAGCATTCGTCAAACTGTTCGTTTTACTACTTGGGATTCAAATATCCAATTATCCTTAGC
ATTTATTGTTAACGCCCTTCTTTTAATCATGGGGGTTGCTGTTTTCAAGACTGGTGCTGTTCAAG
ATAGTTCTTTCTTCGGTTTATATGATGCCCTGAATAACACTTCTATGCTTAGTAATCCAATCTTA
ATTGCGGTTGCTAAATCTGGTGTATTGTCAACTTTATTTGCAGTTGCTTTACTTGCCTCTGGTCA
AAATTCAACTATTACTGGAACATTAACCGGTCAAGTTATCATGGAAGGTTTCATTCATATGCGGA
TGCCTTTATGGGCACGACGGTTAGTTACCCGGATTATTTCCGTTATTCCAGTTATTGCTTGTGTT
GCTATGACGAGTGGTGAGAATACCATCCAACAACACACCGCCTTGAACCTTTTAATGGAAAATTC
ACAGGTATTCTTAGCTTTTGCCCTTCCATTCTCAATGTTGCCATTATTAATGATGACTAACAGTG
AGGTTGAAATGGGTGAATTTAAGAATAGTGGCTGGGTTAAGGCTTGCGGTTGGATCTCTGTAATT
GCCCTTACCTTCTTAAATCTCTACAACCTTCCTGCAACTTATGAAGGTTTTGGTATTTGGGCAAA
AGGGACATCCGATGTTCTCGCATATATCACGATTATTGTCATTCTTGCTCTTCTTATCTGGACTT
GTGTTGAACTCTACAAAGGGGATAAGCGCTTTGCTGCTGAAGGTAAAGGATTCGGACAACGTGAA
GCTCAAATGAAAGATTCAGCTGTAGAAGATTAA
Intervening sequence
(SEQ ID NO: 62)
AATAAAAACCTGGAAGCAATGCTCATGTACGGTAAAGAAGCAAGCAACCGAAAACAATAGATAGA
CCGCCAGCACTACACTATTCCGAACCAAAGACCAAAAGATAAGCATTTTGAGAAAATCTAAACTT
TTGGATTTTCCTACAATGCCGACTACGGCGGAATCCCTCCCACTCCTTATATATTTCTTTCTGTA
TACATTAAATTTGTATTTAGTAAAATGCAGACAACACCACGGATCGGCTTTTGGCTGGACAATTC
CAACCAAACACCGCAGCAGACAGTAGAAACCATTCTGAACGTTAGGAAGCCGGTATGATTGTTAC
ATATAAGGGGAAGAAAAATTTCTTTTAGATACTTGTTTTCCTAAAACTGATGTGATATAATAATT
CAATTCCAGAAAAGGAGTAAAAAATATGCGGCAAGTTATTCTTAAATAAAACTATAATCAAATAG
TGGGAACAAAGGATTATGATAGTCCCTTTTGTAGGGGCTTAGTTTTTTGTACCCAATTTAAGAAT
ACTTTTGCCTTATCAATTTTGACATATCCCCAAAAACAGCACTCACAAACAGGTGTATGCTGTAT
ATGTGTATGTCCGCAAATTATCATCCCCAGTGGTAAAAGTATTTTACTGCTGGGGATTTTTATGC
CCTTCGGGGCAGTAAAGGGAGGACAATCAC
tetW CDS
(SEQ ID NO: 63)
ATGAAAATAATCAATATTGGAATTCTTGCCCATGTAGACGCTGGAAAGACGACCTTGACGGAGAG
CCTGCTATATGCCAGCGGAGCCATTTCAGAACCGGGGAGCGTCAAAAAAGGGACAACGAGGACGG
ACACCATGTTTTTGGAGCGGCAGCGTGGGATTACCATTCAAGCGGCAGTCACTTCCTTCCAGTGG
CACAGATGTAAAGTCAACATTGTGGATACGCCCGGCCACATGGATTTTTTGGCGGAGGTGTACCG
CTCTTTGGCTGTTTTAGATGGGGCCATCTTGGTGATCTCCGCTAAAGATGGCGTGCAGGCCCAGA
CCCGTATTCTGTTCCATGCCCTGCGGAAAATGAACATTCCCACCGTTATCTTTATCAACAAGATC
GACCAGGCTGGCGTTGATTTGCAGAGCGTGGTTCAGTCTGTTCGGGATAAGCTCTCCGCCGATAT
TATCATCAAGCAGACGGTGTCGCTGTCCCCGGAAATAGTCCTGGAGGAAAATACCGACATAGAAG
CATGGGATGCGGTCATCGAAAATAACGATAAATTATTGGAAAAGTATATCGCAGGAGAACCAATC
AGCCGGGAAAAACTTGTGCGGGAGGAACAGCGGCGGGTTCAAGACGCCTCCCTGTTCCCGGTCTA
TTATGGCAGCGCCAAAAAGGGCCTTGGCATTCAACCGTTGATGGATGCGGTGACAGGGCTGTTCC
AACCGATTGGGGAACAGGGGAGCGCCGCCCTATGCGGCAGCGTTTTCAAGGTGGAGTATACAGAT
TGCGGCCAGCGGCGTGTCTATCTACGGCTATACAGCGGAACGCTGCGCCTGCGGGATACGGTGGC
CCTGGCCGGGAGAGAAAAGCTGAAAATCACAGAGATGCGTATTCCATCCAAAGGGGAAATTGTTC
GGACAGACACCGCTTATCCGGGTGAAATTGTTATCCTTCCCAGCGACAGCGTGAGGTTAAACGAT
GTATTAGGGGACCCAACCCGGCTCCCTCGTAAAAGGTGGCGTGAGGACCCCCTCCCCATGCTGCG
GACGTCGATTGCGCCGAAAACGGCAGCGCAAAGAGAACGGCTGCTGGACGCTCTTACGCAACTTG
CGGATACTGACCCGCTTTTGCGCTGCGAGGTGGATTCCATCACCCATGAGATCATTCTTTCTTTT
TTGGGCCGGGTGCAGTTGGAGGTTGTTTCCGCTTTGCTGTCGGAAAAATACAAGCTTGAAACAGT
GGTAAAGGAACCCACCGTCATTTATAAGGAGCGGCCGCTCAAAGCAGCCAGCCACACCATCCATA
TCGAGGTGCCGCCCAACCCGTTTTGGGCATCCATCGGACTGTCTGTTACACCACTCCCGCTTGGC
TCCGGTGTACAATACGAGAGCCGGGTTTCGCTGGGATACTTGAACCAGAGTTTTCAAAACGCTGT
CAGGGATGGTATCCGTTACGGGCTGGAGCAGGGCTTGTTCGGCTGGAACGTAACGGACTGTAAGA
TTTGCTTTGAATACGGGCTTTATTACAGTCCGGTCAGCACGCCGGCGGACTTCCGCTCATTGGCC
CCGATTGTATTGGAACAGGCATTGAAGGAATCAGGGACGCAACTGCTGGAACCTTATCTCTCCTT
CACCCTCTATGCGCCCCGGGAATATCTTTCCAGGGCTTATCATGATGCACCGAAATACTGTGCCA
CCATCGAAACGGTCCAGGTAAAAAAGGATGAAGTTGTCTTTACTGGCGAGATTCCCGCCCGCTGT
ATACAGGCATACCGTACTGATCTGGCCTTTTACACCAACGGGCAGAGCGTATGCCTTACAGAACT
GAAAGGGTATCAGGCCGCTGACGGCAAGCCAGTCATCCAGCCCCGCCGTCCAAACAGCCGCCTGG
ACAAGGTGCGCTATATGTTTCAGAAGATAATGTAA
Intervening sequence
(SEQ ID NO: 64)
CTGCTTCCAGTTTTTTATTTTTGTAACGATTCTTTCACAAAATGTTCATACTTGAACGGTATAGT
ATAGACAATCCCTAATATAAATAAACCCCTATTTTTCTTCTATACTATCCCCCTCAAAATAGCTA
TGAAGAAATACGAAAAGTGGTCGGCCCATAATGAGTCGGCCATTTTTTAATACATCTAGGCTCAC
CATGGTATAATAAGGAACTAGATATATTACTCATTAAGTATGAGGTGAAATCA
Hypothetical protein coding sequence CDS
(SEQ ID NO: 65)
TTGAATAATAAAGATAATAATCAAAATAATAATAAGCGGCGACCTACTCGGGTTGAGCTTTATGA
CACTCCTAAGCCAGGAAAAGAAAAGAAAAAACGTTCTTTCTTTAATCGTCAGCCTAAACAGACTA
CCCCTAACCATAAACATAAGACAGCATCCCCTAACAAGAAAAAGAATCCATATGGAATTTCACGT
CGGATGTTCAAGGTCATTTTAGGCTGCCTATTAGTTATCTTAATTGCAACCATTGTTATTTTCTT
AGTTAAACAACAGGACCCTGTCAATGTTGATAGCAATGACACTACTGTCTCCACAAGCACTTCAT
CACGCAAGAAGCATAAAAAGAGCAGCAGTAGCAGTCATAAGCATCGTTCAAGTTCCAATGACAGT
GATGACAATGATACTCAAGATACTTATAGTATTTCAACTCCTAGTCGTTCGGATAACACGCCTCA
ATCAAGTCATCCAACTAACCAACAACCAACTACTAACTCTTCTTCAACTCCACAACAAAACCAAC
CATCGCAGGAAGAACACCATGATCAAGGCAACAGCCAACAACAATCTACTTCACAACCAGCAAGC
AGCAATTCTGGGTCACAGCCTAATCAAAGTTCGCAAAGTACTAGTTCCCAGTCAAACCCGCAACC
AGTTCAACCTCATTCTCAAAATAATCAGCATTAA
Intervening sequence
(SEQ ID NO: 66)
TTTGGAATAAAGGGGTTGTCAATTAAAGTGGGCGTGCTATAATCAAGACAATCGATTGAAGGATA
TGCACCTTCAAAGTCCTCTACAGAGAGTAAGTGCCTTGAGTGGAAGCACTTGTGAGTGACAATGA
TGGTTGTACCACCTTCCCTTATTATTAGCCGAGATAAGGCTAGTCGGATCGTGTCCGTTACCGCA
CCTTAGAGAGGACATTACTAGTGTGTCCTAAAGATGGGTGGAACCACGCTAATTTTTGTTAATTG
ACGTCCCTGATACTTTCAACATTGAAAGTATCAGGGACGTTTTTATTTATATTAAAGGAGAGATT
TCT
thrS CDS
(SEQ ID NO: 67)
ATGGCTCAGGTTGCTGTTATGTCCCCAGATGGATCAGTTAAGAAGATCGATCGGGATTCACAAGA
AAGTTTAGAAGCATTACGTAAGCTTTCTGCATTAATGTTAAAGGCTGCATTAAAACAAGAATTTA
AGGGTATTCGACTTGGTGAAGCTGTCGCTGACGAAGATGGTTTCCACGTTGATTCTGATAAAGAT
AATCAACAAGTTTCTGCCGACGAATTACCAGCTCTTGAAGACGTAATCAAGGGAATGGCAAAGAA
CGATGTTAAGGTAGAATTTGTTGAAGTACCTGTAGAGGAAGCACTTGCCGAAGTAAAAGATGATC
GTTTCTCAACTGAATTGATCAATGAAAATGCTAAAGACGGCAAAGTAGCAATGTACCAACTTGGT
GATGTCAAAGCTGTTGCAGATGATGACATTCTTTTATATGGTAACGTTGTTAAAAACTTACGCCT
TCTTTCTGTTGCTGGTGCTTACTGGAAAGGTATGTCTTCAAATCCAATGCTTCAACGGATTTACG
GAACTGTCTTCTACAAGAAGGACGCATTAGAAGAAGACTTAAAGAAACGTCAAGAAGCTAAGGAA
CGTGACCACCGTGTTATCGGTAACCAACTCGACCTCTTCTTTGTTGATCCTAAGGTTGGTGCCGG
TTTACCATACTGGTTACCAAAAGGTGCTACTATTCGCCGGACAATCGAACGTTACATCATTGACC
GGGAAGTTGCCGATGGTTACCAACACGTTTATACTCCAGTCCTAATGAATCTTGATGCCTACAAG
ACTTCTGGTCACTGGGAACACTACCGCGACGATATGTTCCCACCAATGGACATGGGTGATGGCGA
AATGCTTGAATTACGGCCAATGAACTGCCCAAGCCATATTCAAGTTTACAAGCACCATATTCGTT
CATACCGTGATCTCCCATTACGAATTGCTGAACTTGGTATGATGCACCGTTATGAAAAATCAGGT
GCTCTTTCTGGTCTTCAACGTGTTCGTGAAATGACTTTGAACGATGGCCACACTTTCGTTACCCT
TGACCAAATTCGTTCTGAATTCGCAAAGATTTTGAAGTTGATCATGAGCGTTTACGAAGACTTTG
ATATTACTGACTACAGCTTCCGTCTTTCTCTTCGTGACCCTAAGAACGTTAAGAAGTACTATGCT
AATGACGAAATGTGGGAAAAATCCCAATCAATGTTGAAATCAGCAATGGACGATCTTAACCTTGA
TTATTATGAAGCTGAAGGTGAAGCTGCCTTCTATGGTCCAAAACTTGATATTCAAACTAAGACTG
CTCTTGGGAATGACGAAACAATGTCAACTATTCAACTTGACTTTATGCTTCCAGAACGATTCGGA
CTTTCCTACGTTGGTCAAGACGGTAAAGAACATCAACCAGTTATGATTCACCGTGGTGTTGTTGG
AACAATGGAACGGTTCATGGCTTACTTAACAGAAATTTACAAGGGTGCATTCCCAACTTGGTTAG
CCCCAGAACAAGTCCACATTATCCCTGTTAACGAAGAAGCCCATGGTGAATACGCAGATAACCTC
GCTAAGAAGATGAAGGCTGCTAACATTCGGGTTAATGTTGACCACCGAAATGAAAAGATGGGCTA
CAAGATTCGTGAAGCTCAAACACAAAAGGTTCCATACACTCTTGTTGTTGGAGACGATGAAAAGA
ATAACAATGGTGTATCTGTTCGTAAGTACGGTGAAAAGGAACAAAATGAAATGAGTCAAGAAGCA
TTTATGAATGAAATTCTTGAAGATATTGCTTCTTACTCCCGTGAAAAGTAA
> L. reuteri PTA-126788 (strain 3632) tetW sequence
along with flanking gene sequences
tdh CDS
(SEQ ID NO: 68)
TTAAGCTTCTTCGCCGCCAACCTTAACAACAGCTTTTGAAACCTTACCTAATTTACCACTAACAA
AGTCTTCAACACCATCAAGGTCTAATTCGTGTGAGAAGAGTGGTAGTGGGTCAACAACACCAGAA
GAAAGAAGTGCAATTGAGTCTTCAAAAGTGTAAGGGTTAATGAAGGCCCCTTGAATTGTAAGTTG
TTTTTGGAATACATCGTAAGTGTTTACTGAGAACTTGTCATCAGGGTTACCAACACCAAACATTA
ATACTTGAGCACCACGAGCTGCAGCTGCAAGTGCTTGTTCTTGAGTAGCTGGCAAACCAACCGCT
TCAACAACGATGTCATATGCATCAGCAGGAATTTCTTCCTTAGTAGTGTTGATAGTCTTAACGCC
GAAGTGCTTCCGGTTGTTTTCCAACTTTTCATCTGAACGACCAGCTAAAGTAACTTCGTGAATAC
CACGAGCCTTCAAGATTTGTGCAAATAATTGACCTTCAAAACCATCACCTAATACTAAAGCCTTT
TGGTATGGGTGAGTTTCAAGTAAGTCAACACCGTGCATTGCACATGAAATTGGTTCAACAACGGC
AGCTGCCTTTAATGAAACATCATCAGGAATTGGGTAAACAACTTTTGCAGGAGCAGTAAAGTATT
CTTCAAAACCACCGTTACGGGTAACACCAACAGCATCAAGGTGTTCACATAATTCTGGGCGTTGA
GTCCGACAATACTTACATTGACCACAGTAGATGTTAGGGTCAACGGTTACACGATCACCTGGCTT
AACGTTAGTAACTTCTGAACCAACTTTTGTAACTACCCCTGAGTTTTCATGACCTAAAACGATAG
GAGGAACAGCTGATGCAGATCCCGGAAGACCGGCATAGAGCGCTTTATCAGTACCACAAATACCA
GCGTAGGCAGTATGAATTAAGACTTCATCAGGCTTAATTTCAGGTTCTTTAATATCTTCAATTTC
GAGTTGTTTTTTACCTGTTAAAACAAGTGCCTTCAT
intervening sequence
(SEQ ID NO: 69)
AATTAAAATACCTCTCTTGTGAATTTTATCATTTGTCAAGTGATAGTATAGTGTGAAAACGCCTT
AATTGTCAAACGTCCAACAATTATCTCACATTCTTTTCGCCATCATAAAACGTTAAACTCAGCAA
ATCCGAACCTAAATAAATCCCGGTATAGAAGCATTTTCTAAAAAAGTGCAATTTTTAAATATTTT
TTTCTTCACTAAGTTTATGCTATTTTTTTGTTATCACATAAATGGTTTTACTTTCATTTTTTAAA
TTACATAATTTCACAAGGCTTTTTATCCTATTTAGCGTTTTACATCAAGAAATAAAATGTTTCTA
CATATAAAGTAATAAATTCTAAATTTATATAGAAAATTTCTCCAACTTTCAGAAAGCGCTTAACT
ACCATTAGGCACGCCAAAACTTATCTCCAACCGCTCCTCAAAATCGCCCAAATGGCCGTTATAAC
GGCTATTTTCCATTGCAGGATATATTTTTTTATGTTACCCTAACAAAGTATTCGAACTTAGTTAA
TTATGATTTCCAATCTAATTAACAAAACTCAAACGAAAGGTGGATTTGGC
mntH CDS
(SEQ ID NO: 70)
GTGGACGACACAAAGAATCAACACCGGAAGCATAAACTTATTGAATATGCTAACGGTAAATCACT
AGAGGAAATCAACGGAACAGTTGAAGTTCCTCGTGGAAAAGGCTTCTGGCGAACATTATTCGCTT
ACTCTGGTCCCGGTGCATTAGTTGCTGTGGGTTACATGGATCCAGGTAACTGGTCAACTTCAATT
ACTGGTGGACAGAGTTTCCAATATACCTTAATGACTACTATCTTGATTTCAAGTTTGATTGCGAT
GTTGCTTCAATACATGGCGGCTAAACTCGGAATCGTGAGCCAAATGGACCTTGCTCAGGCAACAC
GGGCACGTACCGGTAAAGCATTAGGTATTATCTTATGGATTATGACTGAGTTGGCGATTATGGCT
ACTGATATCGCTGAAGTTATCGGGGCTGCTATTGCTTTAAACTTACTGTTCCACATTCCGTTGAT
CCCATCTGTATTTATTACTGTTCTTGATGTTTTAGTACTGCTATTATTAACCAAGATCGGATTCC
GGAAGATTGAAGCAATTGTTGCATGTTTGATCTTGGTAATCTTGTTTGTTTTTGCTTACCAAGTT
GCCTTATCTAACCCTAACTGGGGTGGCGTATTTATGGGTCTCCTTCCATCAGCTAAGGCAATTGC
CCAACATCCAGAAATTAGTGGTATTACTCCATTAACTGGATCATTAGGTATTATCGGTGCGACTG
TTATGCCTCACAACCTATATCTCCACTCAGCAATTTCTCAAACTCGGAAGATTGACCACAATGAT
CTTGATAGCATTCGTCAAACTGTTCGTTTTACTACTTGGGATTCAAATATCCAATTATCCTTAGC
ATTTATTGTTAACGCCCTTCTTTTAATCATGGGGGTTGCTGTTTTCAAGACTGGTGCTGTTCAAG
ATAGTTCTTTCTTCGGTTTATATGATGCCCTGAATAACACTTCTATGCTTAGTAATCCAATCTTA
ATTGCGGTTGCTAAATCTGGTGTATTGTCAACTTTATTTGCAGTTGCTTTACTTGCCTCTGGTCA
AAATTCAACTATTACTGGAACATTAACCGGTCAAGTTATCATGGAAGGTTTCATTCATATGCGGA
TGCCTTTATGGGCACGACGGTTAGTTACCCGGATTATTTCCGTTATTCCAGTTATTGCTTGTGTT
GCTATGACGAGTGGTGAGAATACCATCCAACAACACACCGCCTTGAACCTTTTAATGGAAAATTC
ACAGGTATTCTTAGCTTTTGCCCTTCCATTCTCAATGTTGCCATTATTAATGATGACTAACAGTG
AGGTTGAAATGGGTGAATTTAAGAATAGTGGCTGGGTTAAGGCTTGCGGTTGGATCTCTGTAATT
GCCCTTACCTTCTTAAATCTCTACAACCTTCCTGCAACTTATGAAGGTTTTGGTATTTGGGCAAA
AGGGACATCCGATGTTCTCGCATATATCACGATTATTGTCATTCTTGCTCTTCTTATCTGGACTT
GTGTTGAACTCTACAAAGGGGATAAGCGCTTTGCTGCTGAAGGTAAAGGATTCGGACAACGTGAA
GCTCAAATGAAAGATTCAGCTGTAGAAGATTAA
intervening sequence
(SEQ ID NO: 71)
AATAAAAACCTGGAAGCAATGCTCATGTACGGTAAAGAAGCAAGCAACCGAAAACAATAGATAGA
CCGCCAGCACTACACTATTCCGAACCAAAGACCAAAAGATAAGCATTTTGAGAAAATCTAAACTT
TTGGATTTTCCTACAATGCCGACTACGGCGGAATCCCTCCCACTCCTTATATATTTCTTTCTGTA
TACATTAAATTTGTATTTAGTAAAATGCAGACAACACCACGGATCGGCTTTTGGCTGGACAATTC
CAACCAAACACCGCAGCAGACAGTAGAAACCATTCTGAACGTTAGGAAGCCGGTATGATTGTTAC
ATATAAGGGGAAGAAAAATTTCTTTTAGATACTTGTTTTCCTAAAACTGATGTGATATAATAATT
CAATTCCAGAAAAGGAGTAAAAAATATGCGGCAAGTTATTCTTAAATAAAACTATAATCAAATAG
TGGGAACAAAGGATTATGATAGTCCCTTTTGTAGGGGCTTAGTTTTTTGTACCCAATTTAAGAAT
ACTTTTGCCTTATCAATTTTGACATATCCCCAAAAACAGCACTCACAAACAGGTGTATGCTGTAT
ATGTGTATGTCCGCAAATTATCATCCCCAGTGGTAAAAGTATTTTACTGCTGGGGATTTTTATGC
CCTTCGGGGCAGTAAAGGGAGGACAATCAC
tetW CDS
(SEQ ID NO: 72)
ATGAAAATAATCAATATTGGAATTCTTGCCCATGTAGACGCTGGAAAGACGACCTTGACGGAGAG
CCTGCTATATGCCAGCGGAGCCATTTCAGAACCGGGGAGCGTCAAAAAAGGGACAACGAGGACGG
ACACCATGTTTTTGGAGCGGCAGCGTGGGATTACCATTCAAGCGGCAGTCACTTCCTTCCAGTGG
CACAGATGTAAAGTCAACATTGTGGATACGCCCGGCCACATGGATTTTTTGGCGGAGGTGTACCG
CTCTTTGGCTGTTTTAGATGGGGCCATCTTGGTGATCTCCGCTAAAGATGGCGTGCAGGCCCAGA
CCCGTATTCTGTTCCATGCCCTGCGGAAAATGAACATTCCCACCGTTATCTTTATCAACAAGATC
GACCAGGCTGGCGTTGATTTGCAGAGCGTGGTTCAGTCTGTTCGGGATAAGCTCTCCGCCGATAT
TATCATCAAGCAGACGGTGTCGCTGTCCCCGGAAATAGTCCTGGAGGAAAATACCGACATAGAAG
CATGGGATGCGGTCATCGAAAATAACGATAAATTATTGGAAAAGTATATCGCAGGAGAACCAATC
AGCCGGGAAAAACTTGTGCGGGAGGAACAGCGGCGGGTTCAAGACGCCTCCCTGTTCCCGGTCTA
TTATGGCAGCGCCAAAAAGGGCCTTGGCATTCAACCGTTGATGGATGCGGTGACAGGGCTGTTCC
AACCGATTGGGGAACAGGGGAGCGCCGCCCTATGCGGCAGCGTTTTCAAGGTGGAGTATACAGAT
TGCGGCCAGCGGCGTGTCTATCTACGGCTATACAGCGGAACGCTGCGCCTGCGGGATACGGTGGC
CCTGGCCGGGAGAGAAAAGCTGAAAATCACAGAGATGCGTATTCCATCCAAAGGGGAAATTGTTC
GGACAGACACCGCTTATCCGGGTGAAATTGTTATCCTTCCCAGCGACAGCGTGAGGTTAAACGAT
GTATTAGGGGACCCAACCCGGCTCCCTCGTAAAAGGTGGCGTGAGGACCCCCTCCCCATGCTGCG
GACGTCGATTGCGCCGAAAACGGCAGCGCAAAGAGAACGGCTGCTGGACGCTCTTACGCAACTTG
CGGATACTGACCCGCTTTTGCGCTGCGAGGTGGATTCCATCACCCATGAGATCATTCTTTCTTTT
TTGGGCCGGGTGCAGTTGGAGGTTGTTTCCGCTTTGCTGTCGGAAAAATACAAGCTTGAAACAGT
GGTAAAGGAACCCACCGTCATTTATAAGGAGCGGCCGCTCAAAGCAGCCAGCCACACCATCCATA
TCGAGGTGCCGCCCAACCCGTTTTGGGCATCCATCGGACTGTCTGTTACACCACTCCCGCTTGGC
TCCGGTGTACAATACGAGAGCCGGGTTTCGCTGGGATACTTGAACCAGAGTTTTCAAAACGCTGT
CAGGGATGGTATCCGTTACGGGCTGGAGCAGGGCTTGTTCGGCTGGAACGTAACGGACTGTAAGA
TTTGCTTTGAATACGGGCTTTATTACAGTCCGGTCAGCACGCCGGCGGACTTCCGCTCATTGGCC
CCGATTGTATTGGAACAGGCATTGAAGGAATCAGGGACGCAACTGCTGGAACCTTATCTCTCCTT
CACCCTCTATGCGCCCCGGGAATATCTTTCCAGGGCTTATCATGATGCACCGAAATACTGTGCCA
CCATCGAAACGGTCCAGGTAAAAAAGGATGAAGTTGTCTTTACTGGCGAGATTCCCGCCCGCTGT
ATACAGGCATACCGTACTGATCTGGCCTTTTACACCAACGGGCAGAGCGTATGCCTTACAGAACT
GAAAGGGTATCAGGCCGCTGACGGCAAGCCAGTCATCCAGCCCCGCCGTCCAAACAGCCGCCTGG
ACAAGGTGCGCTATATGTTTCAGAAGATAATGTAA
intervening sequence
(SEQ ID NO: 73)
CTGCTTCCAGTTTTTTATTTTTGTAACGATTCTTTCACAAAATGTTCATACTTGAACGGTATAGT
ATAGACAATCCCTAATATAAATAAACCCCTATTTTTCTTCTATACTATCCCCCTCAAAATAGCTA
TGAAGAAATACGAAAAGTGGTCGGCCCATAATGAGTCGGCCATTTTTTAATACATCTAGGCTCAC
CATGGTATAATAAGGAACTAGATATATTACTCATTAAGTATGAGGTGAAATCA
hypothetical protein CDS
(SEQ ID NO: 74)
TTGAATAATAAAGATAATAATCAAAATAATAATAAGCGGCGACCTACTCGGGTTGAGCTTTATGA
CACTCCTAAGCCAGGAAAAGAAAAGAAAAAACGTTCTTTCTTTAATCGTCAGCCTAAACAGACTA
CCCCTAACCATAAACATAAGACAGCATCCCCTAACAAGAAAAAGAATCCATATGGAATTTCACGT
CGGATGTTCAAGGTCATTTTAGGCTGCCTATTAGTTATCTTAATTGCAACCATTGTTATTTTCTT
AGTTAAACAACAGGACCCTGTCAATGTTGATAGCAATGACACTACTGTCTCCACAAGCACTTCAT
CACGCAAGAAGCATAAAAAGAGCAGCAGTAGCAGTCATAAGCATCGTTCAAGTTCCAATGACAGT
GATGACAATGATACTCAAGATACTTATAGTATTTCAACTCCTAGTCGTTCGGATAACACGCCTCA
ATCAAGTCATCCAACTAACCAACAACCAACTACTAACTCTTCTTCAACTCCACAACAAAACCAAC
CATCGCAGGAAGAACACCATGATCAAGGCAACAGCCAACAACAATCTACTTCACAACCAGCAAGC
AGCAATTCTGGGTCACAGCCTAATCAAAGTTCGCAAAGTACTAGTTCCCAGTCAAACCCGCAACC
AGTTCAACCTCATTCTCAAAATAATCAGCATTAA
intervening sequence
(SEQ ID NO: 75)
TTTGGAATAAAGGGGTTGTCAATTAAAGTGGGCGTGCTATAATCAAGACAATCGATTGAAGGATA
TGCACCTTCAAAGTCCTCTACAGAGAGTAAGTGCCTTGAGTGGAAGCACTTGTGAGTGACAATGA
TGGTTGTACCACCTTCCCTTATTATTAGCCGAGATAAGGCTAGTCGGATCGTGTCCGTTACCGCA
CCTTAGAGAGGACATTACTAGTGTGTCCTAAAGATGGGTGGAACCACGCTAATTTTTGTTAATTG
ACGTCCCTGATACTTTCAACATTGAAAGTATCAGGGACGTTTTTATTTATATTAAAGGAGAGATT
TCT
thrS CDS
(SEQ ID NO: 76)
ATGGCTCAGGTTGCTGTTATGTCCCCAGATGGATCAGTTAAGAAGATCGATCGGGATTCACAAGA
AAGTTTAGAAGCATTACGTAAGCTTTCTGCATTAATGTTAAAGGCTGCATTAAAACAAGAATTTA
AGGGTATTCGACTTGGTGAAGCTGTCGCTGACGAAGATGGTTTCCACGTTGATTCTGATAAAGAT
AATCAACAAGTTTCTGCCGACGAATTACCAGCTCTTGAAGACGTAATCAAGGGAATGGCAAAGAA
CGATGTTAAGGTAGAATTTGTTGAAGTACCTGTAGAGGAAGCACTTGCCGAAGTAAAAGATGATC
GTTTCTCAACTGAATTGATCAATGAAAATGCTAAAGACGGCAAAGTAGCAATGTACCAACTTGGT
GATGTCAAAGCTGTTGCAGATGATGACATTCTTTTATATGGTAACGTTGTTAAAAACTTACGCCT
TCTTTCTGTTGCTGGTGCTTACTGGAAAGGTATGTCTTCAAATCCAATGCTTCAACGGATTTACG
GAACTGTCTTCTACAAGAAGGACGCATTAGAAGAAGACTTAAAGAAACGTCAAGAAGCTAAGGAA
CGTGACCACCGTGTTATCGGTAACCAACTCGACCTCTTCTTTGTTGATCCTAAGGTTGGTGCCGG
TTTACCATACTGGTTACCAAAAGGTGCTACTATTCGCCGGACAATCGAACGTTACATCATTGACC
GGGAAGTTGCCGATGGTTACCAACACGTTTATACTCCAGTCCTAATGAATCTTGATGCCTACAAG
ACTTCTGGTCACTGGGAACACTACCGCGACGATATGTTCCCACCAATGGACATGGGTGATGGCGA
AATGCTTGAATTACGGCCAATGAACTGCCCAAGCCATATTCAAGTTTACAAGCACCATATTCGTT
CATACCGTGATCTCCCATTACGAATTGCTGAACTTGGTATGATGCACCGTTATGAAAAATCAGGT
GCTCTTTCTGGTCTTCAACGTGTTCGTGAAATGACTTTGAACGATGGCCACACTTTCGTTACCCT
TGACCAAATTCGTTCTGAATTCGCAAAGATTTTGAAGTTGATCATGAGCGTTTACGAAGACTTTG
ATATTACTGACTACAGCTTCCGTCTTTCTCTTCGTGACCCTAAGAACGTTAAGAAGTACTATGCT
AATGACGAAATGTGGGAAAAATCCCAATCAATGTTGAAATCAGCAATGGACGATCTTAACCTTGA
TTATTATGAAGCTGAAGGTGAAGCTGCCTTCTATGGTCCAAAACTTGATATTCAAACTAAGACTG
CTCTTGGGAATGACGAAACAATGTCAACTATTCAACTTGACTTTATGCTTCCAGAACGATTCGGA
CTTTCCTACGTTGGTCAAGACGGTAAAGAACATCAACCAGTTATGATTCACCGTGGTGTTGTTGG
AACAATGGAACGGTTCATGGCTTACTTAACAGAAATTTACAAGGGTGCATTCCCAACTTGGTTAG
CCCCAGAACAAGTCCACATTATCCCTGTTAACGAAGAAGCCCATGGTGAATACGCAGATAACCTC
GCTAAGAAGATGAAGGCTGCTAACATTCGGGTTAATGTTGACCACCGAAATGAAAAGATGGGCTA
CAAGATTCGTGAAGCTCAAACACAAAAGGTTCCATACACTCTTGTTGTTGGAGACGATGAAAAGA
ATAACAATGGTGTATCTGTTCGTAAGTACGGTGAAAAGGAACAAAATGAAATGAGTCAAGAAGCA
TTTATGAATGAAATTCTTGAAGATATTGCTTCTTACTCCCGTGAAAAGTAA

During carbohydrate fermentation, Lactobacillus species are known to produce either exclusively L-lactic acid, exclusively D-lactic acid or a racemic mix of L- and D-lactic acid (80). Many commercially used Lactobacillus species produce a racemic mix of L- and D-lactic acid, including the most widely used L. reuteri probiotic strains DSM 17938 and NCIMB 30242 (81-84). Screening for D-lactic acid has gained much attention due to D-lactic acidosis and encephalopathy reported in individuals with short bowel syndrome and intestinal failure (85-87). However, such illnesses have not yet been reported in healthy individuals. Quantification of L- and D-lactic acid showed that our strains produce a racemic mix of L- and D-lactic acid with predominance of L-lactic acid. Consistent with the previous reports, DSM 17938 also produced a racemic mix of D- and L-lactic acid (81). NCIMB 30242, another widely used L. reuteri probiotic strain, also produces a racemic mix of D- and L-lactic acid in a ratio of 9:11 (55). Many clinical studies conducted on DSM 17938 and NCIMB 30242 in infants, children and adults showed no evidence of adverse effects from D-lactic acidosis (26).

Lactobacillus species also possess amino acid decarboxylase activity, which results in production of toxic metabolites such as histamine, tyramine, cadaverine and putrescine. Toxicity from biogenic amines are rare but when reported is mostly associated with histamine and less commonly with tyramine (88-90). Genome analysis showed that our strains do not encode for any known genes encoding for histamine or tyramine production. Analysis of the strains for their ability to produce biogenic amines using decarboxylase media developed by Bover-Cid and Holzapfei (56) showed that our strains are not capable of producing histamine or tyramine. The data clearly suggest that our strains do not produce the two major biogenic amines associated with toxicity in humans—histamine, and tyramine.

Our bioinformatic search identified a CDS predicted to encode arginine deiminase in both L. reuteri PTA-126787 and PTA-126788. Arginine deiminase is a common enzyme present in most lactic acid bacteria and is used to convert arginine into ornithine via citrulline and allows bacteria to adapt to non-optimal stress conditions such as acid, osmotic and temperature stresses (91). Expectedly, a gene encoding arginine deiminase was also present in the genome of the commercially marketed L. reuteri strain DSM 17938 (Accession no. WP_003670382.1). Our bioinformatics analysis showed that the downstream gene ornithine decarboxylase required for putrescine production is absent in the genomes of L. reuteri PTA-126787 and PTA-126788. Consistent with this, in vitro analysis of biogenic amines using decarboxylase media showed that our strains are not capable of producing putrescine using L-ornithine as a substrate. Thus, the presence of arginine deiminase may not result in production of the harmful biogenic amine putrescine.

Studies on the survival properties of probiotic candidates in simulated gastrointestinal conditions are key to our understanding of their safety and efficacy. Probiotic candidates are exposed to a variety of harsh extremes in the gastrointestinal tract, but acidic pH of the stomach and bile salts appear to be the dominant factors determining the survival and growth of probiotic candidates in the gastrointestinal tract. The chicken duodenum has a typical bile salt concentration of 0.175% and likewise, the human duodenum has a bile salt concentration of around 0.3% (92, 93). Both of our L. reuteri strains showed a similar survival profile to that of commercially marketed probiotic L. reuteri DSM 17938 in the presence of 0.3% bile salts. The human and chicken (proventriculus) stomachs have a pH of around 1.5-4.0 (94, 95). Both of our strains showed similar survival at pH 2.5, similar to DSM 17938. Together, these data suggest that the two probiotic candidates possess desirable survival properties in a simulated gastrointestinal environment.

In the present study, broiler chickens (SPF White Leghorn chickens) were used as a model for preliminary screening of L. reuteri PTA-126787 and PTA-126788 strains for gross safety parameters. Our data showed that daily administration of the two L. reuteri strains for 26 days to chickens was safe and did not induce any adverse events. The data provided here serves as a preliminary safety evidence of the two probiotic candidates for potential animal health applications. Future studies will focus on further safety evaluation of the two strains in a rat toxicity model for potential human health applications.

In conclusion, we provide comprehensive genomic, in vitro, and in vivo evidence to support the safety of two novel L. reuteri candidates, PTA-126787 and PTA-126788. These findings would serve as the basis for designing future studies to establish efficacy in humans as well as animals.

### REFERENCES

1. FAO/WHO. Guidelines for the evaluation of probiotics in food report of a joint FAO/WHO working group on drafting guidelines for the evaluation of probiotics in food. London Ontario, Canada: World Health Organization. 2002.
2. Hill C, Guarner F, Reid G, Gibson G R, Merenstein D J, Pot B, et al. Expert consensus document. The International Scientific Association for Probiotics and Prebiotics consensus statement on the scope and appropriate use of the term probiotic. Nat Rev Gastroenterol Hepatol. 2014; 11(8):506-14.
3. Vandenplas Y, Huys G, Daube G. Probiotics: An update. J Pediatr (Rio J). 2015; 91(1):6-21.
4. Aggarwal J, Swami G, Kumar M. Probiotics and their effects on metabolic diseases: An update. J Clin Diagn Res. 2013; 7(1):173-7.
5. Zoumpopoulou G, Tzouvanou A, Mavrogonatou E, Alexandraki V, Georgalaki M, Anastasiou R, et al. Probiotic features of lactic acid bacteria isolated from a diverse pool of traditional Greek dairy products regarding specific strain-host interactions. Probiotics Antimicrob Proteins. 2018; 10(2):313-22.
6. Beijerinck M W. Sur les ferments lactiques de l'industrie. Archives Neerlandaises des Sciences Exactes et Naturelles. 1901(6):212-43.
7. Bourdichon F, Casaregola S, Farrokh C, Frisvad J C, Gerds M L, Hammes W P, et al. Food fermentations: microorganisms with technological beneficial use. Int J Food Microbiol. 2012; 154(3):87-97.
8. Fuller R. Probiotics in man and animals. J Appl Bacteriol. 1989; 66(5):365-78.
9. Reuter G. Das vorkommen von laktobazillen in lebensmitteln und ihr verhalten im menschlichen intestinaltrakt. ZBL Bak Parasit Infec Hyg I Orig. 1965; 197:468-87.
10. Kandler O, Stetter K-O, Kohl R. Lactobacillus reuteri sp. nov., a new species of heterofermentative lactobacilli. Zentralblatt für Bakteriologie: I Abt Originale C: Allgemeine, angewandte und ökologische Mikrobiologie. 1980; 1(3):264-9.
11. Casas I A, Dobrogosz W J. Validation of the probiotic concept: Lactobacillus reuteri confers broad-spectrum protection against disease in humans and animals. Microbial Ecology in Health and Disease. 2000; 12(4):247-85.
12. Mitsuoka T. The Human Gastrointestinal Tract. In: B. J. B. W, editor. The lactic acid bacteria. Boston, MA: Springer; 1992.
13. Valeur N, Engel P, Carbajal N, Connolly E, Ladefoged K. Colonization and immunomodulation by Lactobacillus reuteri ATCC 55730 in the human gastrointestinal tract. Appl Environ Microbiol. 2004; 70(2):1176-81.
14. Talarico T L, Dobrogosz W J. Chemical characterization of an antimicrobial substance produced by Lactobacillus reuteri. Antimicrob Agents Chemother. 1989; 33(5):674-9.
15. Weizman Z, Ash G, Alsheikh A. Effect of a probiotic infant formula on infections in child care centers: comparison of two probiotic agents. Pediatrics. 2005; 115(1):5-9.
16. Kolodziej M, Szajewska H. Lactobacillus reuteri DSM 17938 in the prevention of antibiotic-associated diarrhoea in children: protocol of a randomised controlled trial. BMJ Open. 2017; 7(1):e013928.
17. Mu Q, Tavella V J, Luo X M. Role of Lactobacillus reuteri in human health and diseases. Front Microbiol. 2018; 9:757.
18. Indrio F, Riezzo G, Raimondi F, Bisceglia M, Cavallo L, Francavilla R. The effects of probiotics on feeding tolerance, bowel habits, and gastrointestinal motility in preterm newborns. J Pediatr. 2008; 152(6):801-6.
19. Jones M L, Martoni C J, Parent M, Prakash S. Cholesterol-lowering efficacy of a microencapsulated bile salt hydrolase-active Lactobacillus reuteri NCIMB 30242 yoghurt formulation in hypercholesterolaemic adults. Br J Nutr. 2012; 107(10):1505-13.
20. Shornikova A V, Casas I A, Mykkanen H, Salo E, Vesikari T. Bacteriotherapy with Lactobacillus reuteri in rotavirus gastroenteritis. Pediatr Infect Dis J. 1997; 16(12):1103-7.
21. Abrahamsson T R, Sinkiewicz G, Jakobsson T, Fredrikson M, Bjorksten B. Probiotic lactobacilli in breast milk and infant stool in relation to oral intake during the first year of life. J Pediatr Gastroenterol Nutr. 2009; 49(3):349-54.
22. Schreiber O, Petersson J, Phillipson M, Perry M, Roos S, Holm L. Lactobacillus reuteri prevents colitis by reducing P-selectin-associated leukocyte- and platelet-endothelial cell interactions. Am J Physiol Gastrointest Liver Physiol. 2009; 296(3):G534-42.
23. Klantschitsch T, Spillmann H, Puhan Z, editors. Lactobacillus reuteri: A Newcomer in Dairy Technology; 1996.
24. US-FDA. GRAS exemption claim and exemption notification for Lactobacillus reuteri DSM 17938. In: FDA U, editor: GRAS Notice (GRN) No. 254; 2008.
25. US-FDA. GRAS exemption claim and exemption notification for Lactobacillus reuteri DSM 17938. GRAS Notice (GRN) No. 410; 2012.
26. US-FDA. GRAS exemption claim and exemption notification for Lactobacillus reuteri NCIMB 30242. GRAS Notice (GRN) No. 440; 2013.
27. US-NDIN. NDI 78 —Lactobacillus reuteri—Original NDI Notification. In: 78 NN, editor. 2000.
28. US-NDIN. NDI Notification 460. RepHresh Pro-B (Lactobacillus reuteri RC-14™ and Lactobacillus rhamnosus GR-1™). NDI Notification 4602000.
29. EFSA panel on Biological Hazards, Ricci A, Allende A, Bolton D, Chemaly M, Davies R et al. Scientific opinion on the update of the list of QPS-recommended biological agents intentionally added to food or feed as notified to EFSA. EFSA Journal. 2017; 15(3):e04664.
30. US-FDA. GRAS exemption claim and exemption notification for Lactobacillus reuteri NCIMB 30242. GRAS Notice (GRN) No. 4092012.
31. Wolf B W, Garleb K A, Ataya D G, Casas I A. Safety and tolerance of Lactobacillus reuteri in healthy adult male subjects. Microbial Ecology in Health and Disease. 1995; 8(2):41-50.
32. Wolf B W, Wheeler K B, Ataya D G, Garleb K A. Safety and tolerance of Lactobacillus reuteri supplementation to a population infected with the human immunodeficiency virus. Food Chem Toxicol. 1998; 36(12):1085-94.
33. Pradhan D, Mallappa R H, Grover S. Comprehensive approaches for assessing the safety of probiotic bacteria. Food Control. 2020; 108:106872.
34. Pariza M W, Gillies K O, Kraak-Ripple S F, Leyer G, Smith A B. Determining the safety of microbial cultures for consumption by humans and animals. Regul Toxicol Pharmacol. 2015; 73(1):164-71.
35. Rychen G, Aquilina G, Azimonti G, Bampidis V, Bastos MdL, Bories G, et al. Guidance on the characterisation of microorganisms used as feed additives or as production organisms. EFSA Journal. 2018; 16(3):e05206.
36. Seemann T. Prokka: rapid prokaryotic genome annotation. Bioinformatics. 2014; 30(14):2068-9.
37. Hyatt D, Chen G-L, LoCascio P F, Land M L, Larimer F W, Hauser L J. Prodigal: prokaryotic gene recognition and translation initiation site identification. BMC Bioinformatics. 2010; 11(1):119.
38. Laslett D, Canback B. ARAGORN, a program to detect tRNA genes and tmRNA genes in nucleotide sequences. Nucleic Acids Res. 2004; 32(1):11-6.
39. Suzek B E, Ermolaeva M D, Schreiber M, Salzberg S L. A probabilistic method for identifying start codons in bacterial genomes. Bioinformatics. 2001; 17(12):1123-30.
40. Kingsford C L, Ayanbule K, Salzberg S L. Rapid, accurate, computational discovery of Rho-independent transcription terminators illuminates their relationship to DNA uptake. Genome Biol. 2007; 8(2):R22.
41. Nawrocki E P, Kolbe D L, Eddy S R. Infernal 1.0: inference of RNA alignments. Bioinformatics. 2009; 25(10):1335-7.
42. Tjaden B. A computational system for identifying operons based on RNA-seq data. Methods. 2020; 176:62-70.
43. Na S I, Kim Y O, Yoon S H, Ha S M, Baek I, Chun J. UBCG: Up-to-date bacterial core gene set and pipeline for phylogenomic tree reconstruction. J Microbiol. 2018; 56(4):280-5.
44. Price M N, Dehal P S, Arkin A P. FastTree 2—approximately maximum-likelihood trees for large alignments. PLoS One. 2010; 5(3):e9490.
45. Emms D M, Kelly S. OrthoFinder: phylogenetic orthology inference for comparative genomics. Genome Biology. 2019; 20(1):238.
46. Sayers E W, Beck J, Bolton E E, Bourexis D, Brister J R, Canese K, et al. Database resources of the National Center for Biotechnology Information. Nucleic Acids Res. 2021; 49(D1):D10-d7.
47. Jain C, Rodriguez-R L M, Phillippy A M, Konstantinidis K T, Aluru S. High throughput ANI analysis of 90K prokaryotic genomes reveals clear species boundaries. Nature Communications. 2018; 9(1):5114.
48. Xie Z, Tang H. ISEScan: automated identification of insertion sequence elements in prokaryotic genomes. Bioinformatics. 2017; 33(21):3340-7.
49. Akhter S, Aziz R K, Edwards R A. PhiSpy: a novel algorithm for finding prophages in bacterial genomes that combines similarity- and composition-based strategies. Nucleic Acids Res. 2012; 40(16):e126.
50. Biswas A, Staals R H, Morales S E, Fineran P C, Brown C M. CRISPRDetect: A flexible algorithm to define CRISPR arrays. BMC Genomics. 2016; 17:356.
51. Liu B, Zheng D, Jin Q, Chen L, Yang J. VFDB 2019: a comparative pathogenomic platform with an interactive web interface. Nucleic Acids Res. 2019; 47(D1):D687-D92.
52. Gupta S K, Padmanabhan B R, Diene S M, Lopez-Rojas R, Kempf M, Landraud L, et al. ARG-ANNOT, a new bioinformatic tool to discover antibiotic resistance genes in bacterial genomes. Antimicrob Agents Chemother. 2014; 58(1):212-20.
53. Bortolaia V, Kaas R S, Ruppe E, Roberts M C, Schwarz S, Cattoir V, et al. ResFinder 4.0 for predictions of phenotypes from genotypes. J Antimicrob Chemother. 2020; 75(12):3491-500.
54. Seemann T. Abricate: mass screening of contigs for antimicrobial and virulence genes. [cited 2018. Available from: https://github.com/tseemann/abricate].
55. Branton W B, Jones M L, Tomaro-Duchesneau C, Martoni C J, Prakash S. In vitro characterization and safety of the probiotic strain Lactobacillus reuteri cardioviva NCIMB 30242. International Journal of Probiotics & Prebiotics. 2011; 6(1):1-12.
56. Bover-Cid S, Holzapfel W H. Improved screening procedure for biogenic amine production by lactic acid bacteria. Int J Food Microbiol. 1999; 53(1):33-41.
57. Ferreira C L, Grzeskowiak L, Collado M C, Salminen S. In vitro evaluation of Lactobacillus gasseri strains of infant origin on adhesion and aggregation of specific pathogens. J Food Prot. 2011; 74(9):1482-7.
58. Eschenbach D A, Davick P R, Williams B L, Klebanoff S J, Young-Smith K, Critchlow C M, et al. Prevalence of hydrogen peroxide-producing Lactobacillus species in normal women and women with bacterial vaginosis. J Clin Microbiol. 1989; 27(2):251-6.
59. NCCLS. Development 578 of in vitro susceptibility testing criteria and quality control parameters; approved 579 guideline 2nd ed. Wayne, PA, USA: NCCLS documents M23-A2 NCCLS; 2001.
60. Singh A K, Hertzberger R Y, Knaus U G. Hydrogen peroxide production by lactobacilli promotes epithelial restitution during colitis. Redox Biol. 2018; 16:11-20.
61. Szkaradkiewicz A K, Stopa J, Karpinski™. Effect of oral administration involving a probiotic strain of Lactobacillus reuteri on pro-inflammatory cytokine response in patients with chronic periodontitis. Arch Immunol Ther Exp (Warsz). 2014; 62(6):495-500.
62. FAO/WHO. Guidelines for the evaluation of probiotics in food report of a joint FAO/WHO working group on drafting guidelines for the evaluation of probiotics in food. London Ontario, Canada: World Health Organization; May 1, 2002.
63. Cadieux P, Wind A, Sommer P, Schaefer L, Crowley K, Britton R A, et al. Evaluation of reuterin production in urogenital probiotic Lactobacillus reuteri RC-14. Appl Environ Microbiol. 2008; 74(15):4645-9.
64. Greppi A, Asare P T, Schwab C, Zemp N, Stephan R, Lacroix C. Isolation and comparative genomic analysis of reuterin-producing Lactobacillus reuteri from the chicken gastrointestinal tract. Front Microbiol. 2020; 11:1166.
65. Lee J Y, Han G G, Choi J, Jin G D, Kang S K, Chase B J et al. Pan-genomic approaches in Lactobacillus reuteri as a porcine probiotic: Investigation of host adaptation and antipathogenic activity. Microb Ecol. 2017; 74(3):709-21.
66. Duar R M, Frese S A, Lin X B, Fernando S C, Burkey T E, Tasseva G, et al. Experimental evaluation of host adaptation of Lactobacillus reuteri to different vertebrate species. Appl Environ Microbiol. 2017; 83(12).
67. Oh P L, Benson A K, Peterson D A, Patil P B, Moriyama E N, Roos S, et al. Diversification of the gut symbiont Lactobacillus reuteri as a result of host-driven evolution. The ISME Journal. 2010; 4(3):377-87.
68. Choi H-J, Lee N-K, Paik H-D. Health benefits of lactic acid bacteria isolated from kimchi, with respect to immunomodulatory effects. Food Science and Biotechnology. 2015; 24(3):783-9.
69. Oliveira L C, Saraiva T D L, Silva W M, Pereira U P, Campos B C, Benevides L J, et al. Analyses of the probiotic property and stress resistance-related genes of Lactococcus lactis subsp. lactis NCDO 2118 through comparative genomics and in vitro assays. PLoS ONE. 2017; 12(4):e0175116.
70. Sandes S, Alvim L, Silva B, Acurcio L, Santos C, Campos M, et al. Selection of new lactic acid bacteria strains bearing probiotic features from mucosal microbiota of healthy calves: Looking for immunobiotics through in vitro and in vivo approaches for immunoprophylaxis applications. Microbiological Research. 2017; 200:1-13.
71. Dlamini Z C, Langa R L S, Aiyegoro O A, Okoh A I. Safety evaluation and colonisation abilities of four lactic acid bacteria as future probiotics. Probiotics Antimicrob Proteins. 2019; 11(2):397-402.
72. Nordeste R, Tessema A, Sharma S, Kovač Z, Wang C, Morales R, et al. Molecules produced by probiotics prevent enteric colibacillosis in pigs. BMC Vet Res. 2017; 13(1):335.
73. Salas-Jara M J, Ilabaca A, Vega M, Garcia A. Biofilm forming Lactobacillus: New challenges for the development of probiotics. Microorganisms. 2016; 4(3):35.
74. Granato D, Bergonzelli G E, Pridmore R D, Marvin L, Rouvet M, Corthésy-Theulaz I E. Cell surface-associated elongation factor Tu mediates the attachment of Lactobacillus johnsonii NCC533 (Lal) to human intestinal cells and mucins. Infect Immun. 2004; 72(4):2160-9.
75. Boekhorst J, de Been M W, Kleerebezem M, Siezen R J. Genome-wide detection and analysis of cell wall-bound proteins with LPxTG-like sorting motifs. J Bacteriol. 2005; 187(14):4928-34.
76. Antelmann H, Scharf C, Hecker M. Phosphate starvation-inducible proteins of Bacillus subtilis: Proteomics and transcriptional analysis. Journal of Bacteriology. 2000; 182(16):4478.
77. Oh J-H, Lin X B, Zhang S, Tollenaar S L, Özçam M, Dunphy C, et al. Prophages in Lactobacillus reuteri are associated with fitness trade-offs but can increase competitiveness in the gut ecosystem. Applied and Environmental Microbiology. 2019; 86(1):e01922-19.
78. Lennon J T, Khatana S A, Marston M F, Martiny J B. Is there a cost of virus resistance in marine cyanobacteria?Isme j. 2007; 1(4):300-12.
79. US-FDA. GRAS Exemption Claim and Exemption Notification for Lactobacillus paracasei ssp. paracasei strain FI 9: GRAS Notice (GRN) No. 840; 2018.
80. Axelsson L. Lactic acid bacteria: Classification and physiology. In: Salminen S, Wright, A. V. and Ouwehand, A., editor. Lactic Acid Bacteria: Microbiological and Functional Aspects. 3 ed. New York: Marcel Dekker; 2004. p. 1-67.
81. Papagaroufalis K, Fotiou A, Egli, Tran L A, Steenhout P. A Randomized double blind controlled safety trial evaluating d-lactic acid production in healthy infants fed a Lactobacillus reuteri-containing formula. Nutr Metab Insights. 2014; 7:19-27.
82. Iino T, Manome A, Okada S, Uchimura T, Komagata K. Effects of sodium acetate on the production of stereoisomers of lactic acid by Lactobacillus sakei and other lactic acid bacteria. J Gen Appl Microbiol. 2001; 47(5):223-39.
83. Saulnier D M, Santos F, Roos S, Mistretta T A, Spinler J K, Molenaar D, et al. Exploring metabolic pathway reconstruction and genome-wide expression profiling in Lactobacillus reuteri to define functional probiotic features. PLoS One. 2011; 6(4):e18783.
84. Kyla-Nikkila K, Hujanen M, Leisola M, Palva A. Metabolic engineering of Lactobacillus helveticus CNRZ32 for production of pure L-(+)-lactic acid. Appl Environ Microbiol. 2000; 66(9):3835-41.
85. Karton M, Rettmer R L, Lipkin E W. Effect of parenteral nutrition and enteral feeding on D-lactic acidosis in a patient with short bowel. JPEN J Parenter Enteral Nutr. 1987; 11(6):586-9.
86. Scully T B, Kraft S C, Carr W C, Harig J M. D-lactate-associated encephalopathy after massive small-bowel resection. J Clin Gastroenterol. 1989; 11(4):448-51.
87. Hudson M, Pocknee R, Mowat N A. D-lactic acidosis in short bowel syndrome—an examination of possible mechanisms. Q J Med. 1990; 74(274):157-63.
88. Becker K, Southwick K, Reardon J, Berg R, MacCormack J N. Histamine poisoning associated with eating tuna burgers. JAMA. 2001; 285(10):1327-30.
89. Ohnuma S, Higa M, Hamanaka S, Matsushima K, Yamamuro W. An outbreak of allergy-like food poisoning. Intern Med. 2001; 40(8):833-5.
90. Miki M, Ishikawa T, Okayama H. An outbreak of histamine poisoning after ingestion of the ground saury paste in eight patients taking isoniazid in tuberculous ward. Intern Med. 2005; 44(11):1133-6.
91. Vrancken G, Rimaux T, Wouters D, Leroy F, De Vuyst L. The arginine deiminase pathway of Lactobacillus fermentum IMDO 130101 responds to growth under stress conditions of both temperature and salt. Food Microbiol. 2009; 26(7):720-7.
92. Van Deest B W, Fordtran J S, Morawski S G, Wilson J D. Bile salt and micellar fat concentration in proximal small bowel contents of ileectomy patients. J Clin Invest. 1968; 47(6):1314-24.
93. Lin J, Sahin O, Michel L O, Zhang Q. Critical role of multidrug efflux pump CmeABC in bile resistance and in vivo colonization of Campylobacter jejuni. Infect Immun. 2003; 71(8):4250-9.
94. Musikasang H, Tani A, H-kittikun A, Maneerat S. Probiotic potential of lactic acid bacteria isolated from chicken gastrointestinal digestive tract. World Journal of Microbiology and Biotechnology. 2009; 25(8):1337-45.
95. Kararli T T. Comparison of the gastrointestinal anatomy, physiology, and biochemistry of humans and commonly used laboratory animals. Biopharm Drug Dispos. 1995; 16(5):351-80.

### Example 4

### Evaluation of L. reuteri Strains Evaluated in a Chronic Alcohol Consumption Model

Chronic alcohol consumption is a leading cause of chronic liver disease worldwide, leading to cirrhosis and hepatocellular carcinoma. Alcoholic liver disease (ALD) is a major cause of morbidity and mortality worldwide and includes a broad spectrum of disorders, ranging from steatosis to severe forms of liver injury such as steatohepatitis, cirrhosis and hepatocellular carcinoma (Gao, B. & Bataller, R. (2011) Gastroenterology 141, 1572-1585; Tsukamoto, H. & Lu, S. C. (2001) FASEB J.

15, 1335-1349; O'Shea, R. S., Dasarathy, S. & McCullough, A. J. (2010) Hepatology 51, 307-328; Stickel, F. & Seitz, H. K. (2010) Best Pract. Res. Clin. Gastroenterol. 24, 683-693; Beer, J I, Arteel, G. E. & McClain, C. J. (2011) Curr. Gastroenterol. Rep. 13, 56-64). There is also alcohol-related damage to other organs, such as the pancreas, heart, kidney, lung and CNS. Further, chronic alcohol consumption is associated with compromised innate and adaptive immune responses to infectious disease.

Chronic alcohol abuse is associated with gut barrier alteration, dysbiosis and immune activation. Alcohol-dependent subjects present with increased intestinal permeability (leaky gut) and altered gut microbiota composition and functionality. This favors the translocation of gut-derived bacterial components, such as lipopolysaccharides (LPS) and peptidoglycan (PGN), from the gut lumen to the systemic circulation and other organs. Bacterial ligands are recognized by Toll-like receptors (TLRs) expressed by immune cells and induce an inflammatory response.

Mucosa-associated invariant T (MAIT) cells play a critical role in antibacterial host defense. Binge-on-chronic alcohol feeding has been shown to lead to a significant reduction in the abundance

of MAIT cells in the barrier tissues (Gu M et al (2021) Alcohol Clin Exp Res 00:1-4; doi:10.1111/acer.14589). Transplantation of fecal microbiota from alcohol fed (AF) mice resulted in a MAIT cell profile aligned to that of AF mouse donor. Antibiotic treatment abolished the MAIT cell differences between AF and PF animals. MAIT cells in the intestine, liver, and lung are perturbed by alcohol use and these changes are partially attributable to alcohol-associated dysbiosis. The changes to the
MAIT cell profile in AF animals are mediated by the gut microbiota, as alcohol dysbiosis transplantation replicated many of the effects of alcohol on MAIT cells in the absence of alcohol exposure.

The previously widely used model for alcoholic liver injury was ad libitum feeding with the Lieber-DeCarli liquid diet containing ethanol for 4-6 weeks. This model, without the addition of a secondary insult, only induces mild steatosis, slight elevation of serum alanine transaminase (ALT) and little or no inflammation (Cohen, J. I L et al (2010) Gastroenterology 139, 664-674; Mandrekar, P. et al (2011) Hepatology 54, 2185-2197; Nath, B, et a. (2011) Hepatology 53, 1526-1537; Hu, M. et al. (2012) Hepatology 55, 437-446; Liangpunsakul, S. et al. (2012) Am. J. Physiol. Gastrointest. Liver Physiol. 302, G515-523; Leung, T. M. et al. (2012) Hepatology 55, 1596-1609). Bertola et al have described a simple mouse model of alcoholic liver injury by chronic ethanol feeding (10-d ad libitum oral feeding with the Lieber-DeCarli ethanol liquid diet) plus a single binge ethanol feeding (Bertola A et al (2013) Nature Protocols 8(3):627-637). This chronic-binge ethanol feeding model achieves high blood alcohol levels, elevation of serum ALT and AST and infiltration of neutrophils. This model can be carried out in a shorter time period compared with previous rodent models of ALD (15 d in this chronic-plus-single-binge model versus 5-7 weeks in previous chronic models), and is easily applicable to multiple research areas including ALD and other organ damage caused by alcohol consumption.

Binge-on-chronic alcohol feeding in animal models shifts gut microbiota diversity and functional capacity. Human and rodent studies have reported that excessive alcohol consumption changes gut microbiota composition, structure, and metabolic function (Leclercq S et al. (2014) Proceedings of the National Academy of Sciences of the United States of America, 111, E4485-E4493, 2014a; Mutlu, E A et al. (2012) American Journal of Physiology-Gastrointestinal and Liver Physiology, 302, G966-G978). Recent studies have shown that alterations in the intestinal immune response as a consequence of alcohol-induced dysbiosis contribute to increased susceptibility to infections including bacterial infections such as Klebsiella pneumonia, and Streptococcus pneumonia.

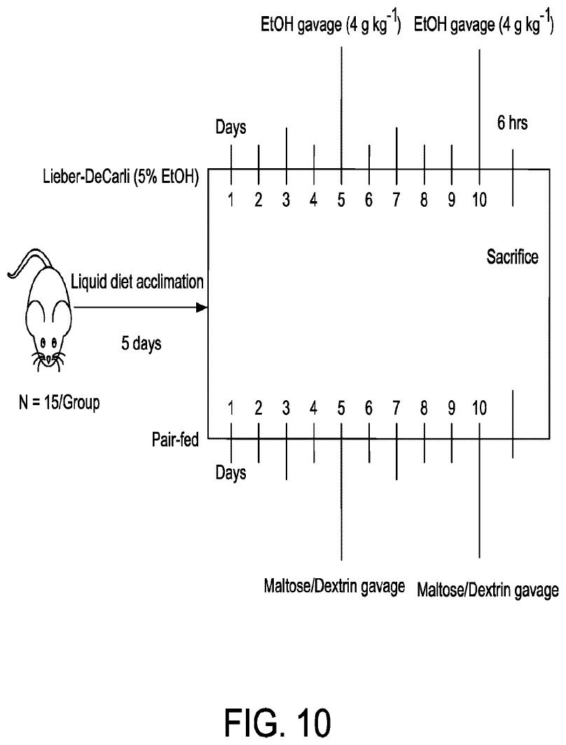

The Lactobacillus reuteri strains strain 3632 (PTA-126788) and strain 3830 (PTA-126787) were evaluated in a chronic-binge ethanol feeding model (Bertola A et al (2013) Nature Protocols 8(3):627-637) for alleviation of alcohol effects. The model-based study is depicted in FIG. 10. Mice were acclimated to Lieber-DeCarli liquid control diet (Bioserv, Flemington, NJ) for 5 days. Afterward, animals were randomized into alcohol-fed (AF; Lieber-DeCarli ethanol [EtOH] liquid diet) and pair-fed (PF) groups (control diet) (n=10/group). (PF: Pair-fed; AF: Alcohol-fed; AFEP: alcohol-fed+ strain 3632 and 3630 probiotic; AFBB: Alcohol-fed+ Blueberry; AFBC: Alcohol-fed+ Broccoli). AF mice were fed with EtOH liquid diet (5.0%, vol/vol) for 10 days and received 4 g/kg body weight EtOH by gavage at day 5 and day 10. Blood alcohol concentrations averaged 200 mg/dl before the binge and reached ˜400 mg/dl post binge (Samuelson D. R. et al (2017) PLOS Pathogens 13(6): e1006426; doi.org/10.1371/journal.ppat.1006426). The amount of control liquid diet for PF mice was adjusted daily according to the food intake of AF mice during the experiment. Mice were sacrificed 24 h following the last binge EtOH administration. The AFEP group was fed 3×10⁹ CFUs/mice/day of L. reuteri 3630 and 3632 (1:1 ratio) for 14 days. AFBC and AFBB groups were fed 2% (w/v) of Broccoli and Blueberry, respectively.

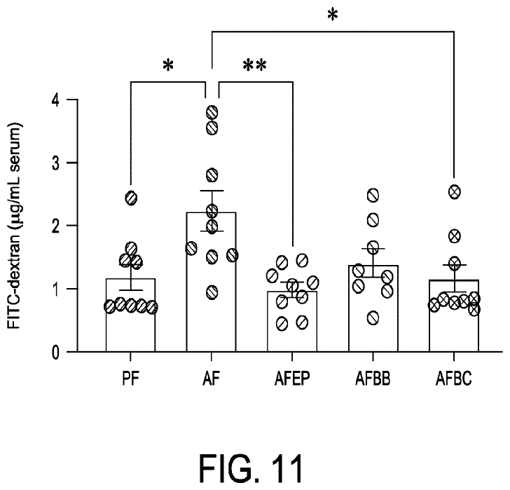

FIG. 10 depicts the protocol for the binge on chronic alcohol murine model utilized in these studies. FIG. 11 provides the effect of various treatments/feed conditions on gut leak in the binge on chronic alcohol murine model. FITC-dextran was gavaged and fluorescence measured in the serum to determine gut leak. Pair fed animals were used as controls and were compared to alcohol fed animals and alcohol fed animals supplemented with L reuteri 3632 and 3630 in their feed. The results clearly demonstrate that a combination of strains 3632 and 3630 led to significantly decreased gut leak, near to or below the levels of control animals.

Administration of a combination of L. reuteri strains strain 3632 (PTA-126788) and strain 3830 (PTA-126787) (in a 1:1 ratio) alleviated the negative effects of alcohol consumption in animals in the chronic alcohol model. Oral administration of L. reuteri 3630 and 3632 protected animals against gut leak as measured by translocation of orally gavaged FITC-dextran into systemic circulation in a binge-on-chronic alcohol model of leaky gut. Intestinal microbial products from alcohol-fed mice have been shown to contribute to intestinal permeability and peripheral immune activation (Samuelson D R et al (2019) Alcohol Clin Exp Res. 43(10):2122-33). Also, alcohol use is associated with intestinal dysbiosis and dysfunctional CD8+ T-cell phenotypes in humans with human immunodeficiency virus (Maffei V J et al J Infect Dis. 2021; 223(6):1029-39).

### Example 5

### Evaluation of L. reuteri Strains on Cytokine Expression in a Chronic-Binge-on Alcohol Model

Chronic inflammatory states, such as IBD and celiac disease and chronic overuse of alcohol have been shown to result in altered or increased intestinal permeability. Intestinal permeability is a recognized feature of several inflammatory and autoimmune diseases affecting the digestive system, including inflammatory bowel disease, Crohn's disease and celiac disease (Bjarnason I et al (1983) Lancet 1:323-5; Hollander D et al (1986) Ann Intern Med 1986; 105:883-5; Vazquez-Roque M I et al (2013) Gastroenterology 144:903-11, e3; D'Incà R et al (1999) Am J Gastroenterol 94:2956-60).

Inflammatory states or conditions can be marked by increased levels of proinflammatory cytokines and/or reduced levels of anti-inflammatory cytokines. The effects of treatment with L. reuteri strains 3630 and 3632 was evaluated in the leaky gut model on the expression of expression of various cytokines.

Proinflammatory cytokines such as IL-6, TNF-α, IFN-γ and IL-1β were evaluated. IL-22 was also assessed as IL-22 is known to regulate intestinal epithelial cell function via promoting epithelial cell regeneration, innate defense and membrane mucus production (Patnaude L et al (2021) Life Sci 271:119195). IL-10 was also determined. IL-22 and IL-10 are anti-inflammatory cytokines.

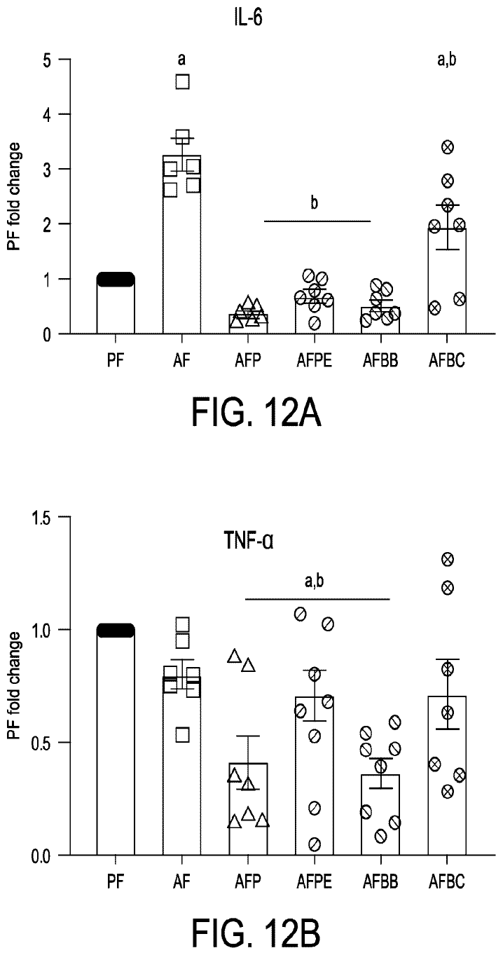

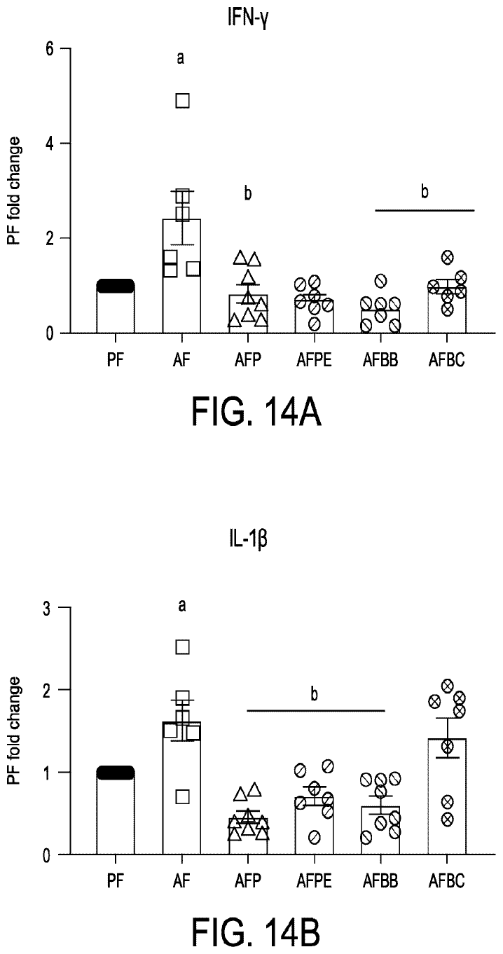

Using animals in the above example binge on chronic alcohol model, the expression of cytokines IL-6, TNF-α, IL-22, IL-10, IFN-γ and IL-1β were determined. The fold change in each of these cytokines in the intestines of animals fed Lieber-DeCarli ethanol (EtOH) liquid diet (denoted AF), pair fed control diet (denoted PF) and alcohol fed mice treated with L reuteri 3632 and 3630 in 1:1 ratio (denoted AFPE) for 10 days is depicted in FIGS. 12, 13 and 14. Animals fed a combination of L. reuteri 3632 and 3630 demonstrated reduced levels of each and all of proinflammatory cytokines IL-6, TNF-α, IFN-γ and IL-1β. Animals fed L. reuteri 3632 and 3630 showed increased levels of IL-22 and of IL-10.

Feeding or administering L. reuteri strains 3630 and 3632 altered inflammation and the inflammatory response in alcohol fed animals, animals with leaky gut or increased intestinal permeability. The strains reduced pro-inflammatory cytokines demonstrating a direct anti-inflammatory effect by the L. reuteri strains. The strains blocked and/or reduced inflammatory responses and protected animals from inflammation and the pro-inflammatory cascade and response. The strains also increased the amount or expression of anti-inflammatory cytokines, particularly including Il-10 and IL-22.

### Example 6

### Lactobacillus reuteri AhR-Activating Metabolite and Polyketide Synthase (PKS) Biosynthetic Cluster

The anti-inflammatory, anti-bacterial and probiotic effects and capabilities of the L reuteri strains 3630 and 3632 are described above. The L. reuteri strains 3630 and 3632 are described and detailed as probiotic strains in Probiotic Compositions Comprising Lactobacillus reuteri Strains and Methods of Use PCT/US2020/016668 filed Feb. 4, 2020, published as WO 2020/163398 Aug. 13, 2020, and corresponding US publications US 2022/0088094 published Mar. 24, 2022 and US 2022/0125860 published Apr. 28, 2022. In particular, as described and demonstrated above, a combination of L. reuteri strains 3630 and 3632 provides significant anti-inflammatory and proinflammatory blocking capability upon administration to animals, including oral administration such as administering in feeds. There are also unique aspects of L. reuteri strain 3632 in terms of anti-inflammatory activity and capability.

The growth kinetics, ability to produce hydrogen peroxide and other properties of the L. reuteri strains, including strains 3632 and 3630 are described above, including in Example 1. Generally, the strains behave similarly in terms of some probiotic properties, including growth kinetics and ability to produce hydrogen peroxide. L. reuteri strain 3632, however, shows some unique properties, including the ability to autoaggregate in liquid media (comparable to that of the well-characterized human probiotic strain L. reuteri ATCC 23272) (FIG. 5). In addition to autoaggregation, L. reuteri 3632 also appears to produce an orange pigment, which resembles beta carotene in color. The other L. reuteri strains evaluated above particularly including L. reuteri strain 3630 (PTA-126787) and the human L. reuteri strain ATCC 23272 and L. acidophilus, produced orange colored pigment. None of the strains is found to be hemolytic on blood agar plates, suggesting that these isolates are less likely to be pathogenic to humans.

Polyketide synthases (PKS) and nonribosomal peptide synthases (NRPS) are secondary metabolites produced by biosynthetic gene clusters (BCGs) that assemble simple molecules, such as acetyl-CoA, into complex metabolites, some of which (i.e., erythromycin) are important to the pharmaceutical industries. A biosynthetic gene cluster (BGC) is a group of genes in bacteria that work together to produce or generate one or more molecules or proteins, or in some instances a protein complex, that provide one or more activity or related activities and/or serve a related or final function. Clustering of a group of genes can permit or enable timed and coordinated synthesis, for example of proteins involved in a pathway. The proteins can be under the control of multiple promoters or transcribed by a single promoter or group of promoters.

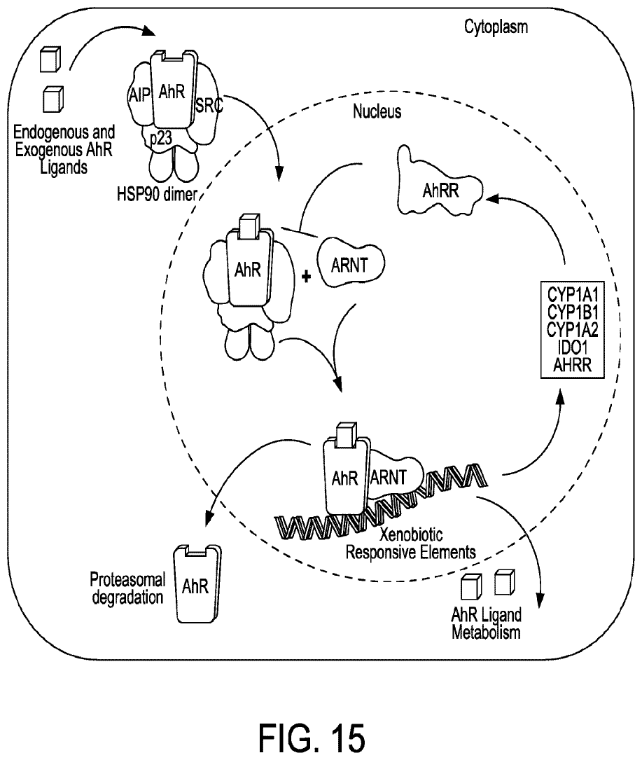

The gut microbiome encodes for several BGCs that produce secondary metabolites that directly interact with the host immune system. Of particular importance is the BGCs that encode for aryl hydrocarbon receptor (AhR)-activating metabolites. AhR is a ligand-activated transcription factor activated by various environmental ligands of plant or microbial origin. AhR recognizes environmental pollutants, dietary compounds (i.e., glucobrassicin and flavonoids), and microbial-derived secondary metabolites (i.e., indole-3-carbinol). Ahr sensing of environmental pollutants and dietary-derived compounds contributes to the intestinal homeostasis between the host and gut microbiota. Upon ligand binding, AhR translocates into the nucleus to induce target gene expressions (FIG. 15; Barrosa A et al (2021) Cellular Mol Immunol 18:259-268). AhR activation by appropriate ligands up-regulates expression of various gene products that either play an essential role in strengthening the epidermal barrier function (such as FLG, LOR and IVL) or help neutralize disease-associated ROS response (NRF2).

The host can recognize microbial metabolites via different pathways, including via pattern recognition receptors and the aryl hydrocarbon receptor (AhR) (Takeuchi O, Akira S (2010) Cell 140:805-820; doi.org/10.1016/j.cell.2010.01.022; Moura-Alves P et al (2014) Nature 512:387-392; doi.org/10.1038/nature13684). The role of AhR has been extensively studied in relation to metabolism of environmental toxins, but the focus has recently shifted to its role in modulation of the adaptive and innate immune system. AhR is a ligand activated transcription factor that plays a key role in a variety of diseases, including amelioration of intestinal inflammation. Understanding the mechanism by which a bacterium modulates the immune system is critical for applying rational selection strategies for probiotic supplementation.

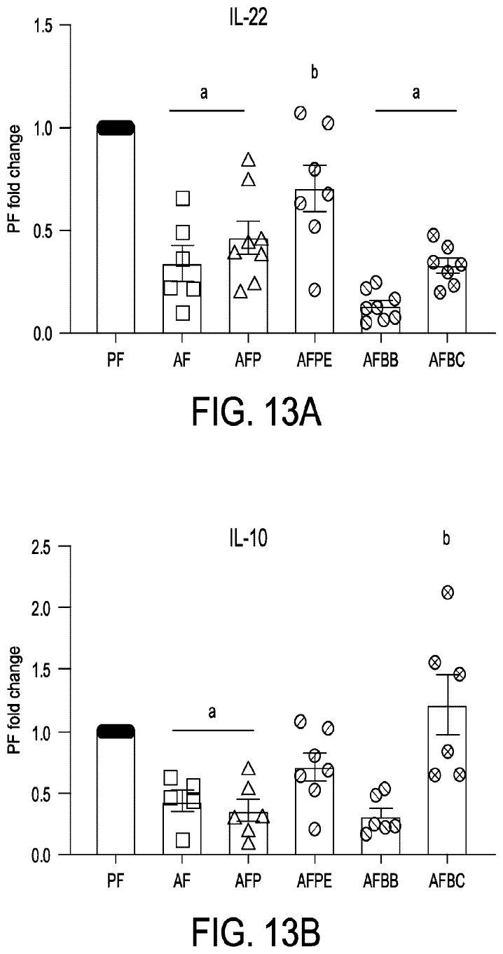

Heterologous and/or optimized expression of PKS provides a step toward the development of next-generation probiotics to prevent and treat disease. The activation of AhR, for example, is important for the production of interleukin-22 (IL-22), which enhances the innate immune response by inducing the production of antimicrobial peptides (Reg3 lectins) to fight off intestinal pathogens and to protect intestinal tissues from inflammation damage by inducing tight junction proteins (Lamas B, Natividad J M, Sokol H. (2018) Mucosal Immunol 11:1024-1038; Monteleone I. et al (2011) Gastroenterology 141:237.e1-248.e1). L. reuteri strains 3630 and 3632 administered in combination are shown above to increase IL-22 expression (Example 5, FIG. 13).

In mammals, 99% of dietary tryptophan is taken up by the host, thereby limiting tryptophan availability to the microbiota to produce AhR ligands. Therefore, identification of specific gut microbes, including L. reuteri strains, that activate AhR independent of tryptophan metabolism is important for developing AhR-mediated biotherapeutic strategies to target intestinal diseases.

The role of AhR has been extensively studied in relation to metabolism of environmental toxins, but the focus has recently shifted to its role in modulation of the adaptive and innate immune system. AhR is a ligand activated transcription factor that plays a key role in a variety of diseases including amelioration of intestinal inflammation. Although initial studies focused on ligands such as polycyclic aromatic hydrocarbons (PAHs), polychlorinated biphenyls (PCBs) and halogenated aromatic hydrocarbons (HAHs), it is now clear that a broad range of dietary, commensal and endogenous, ligands activate AHR (Denison, M. S. et al (2011) Toxicol Sci 124, 1-22; Gutierrez-Vazquez, C. & Quintana, F. J. (2018) Immunity 48, 19-33; Quintana, F. J. & Sherr, D. H. (2013) Pharmacol. Rev. 65, 1148-1161; Lee, H. U. et al (2017) J. Mol. Med. 95, 29-39). Multiple physiological and dietary AHR ligands have been identified, including tryptophan metabolites such as 6-formylindolo[3,2-b]carbazole (FICZ), kynurenine, indigo, indirubin, the pigment curcumin, carotenoids, flavonoids, bilirubin and biliverdin, 2-(1′H-indole-3′-carbonyl)-thiazole-4-carboxylic acid methyl ester (ITE), indoxyl-3-sulfate (I3S), indole-3-carbinol (I3C), gallic acid, prostaglandins and eicosanoids (Barrosa A et al (2021) Cellular Mol Immunol 18:259-268). Additional AHR agonists are produced by the metabolism of commensal microorganisms (Roager, H. M. & Licht, T. R. (2018) Nat Commun 9, 3294; Rothhammer, V. et al. (2018) Nature 557, 724-728).

Ozcam et al have shown that some L. reuteri strains can activate the aryl hydrogen receptor (AhR) and that this activation is associated and correlated with the presence of PKS gene cluster and its metabolite(s) (Ozcam M et al (2019) Appl Environ Microbiology 85(10):e01661-18). Strains that have the PKS biosynthetic gene cluster activate AhR and produce a bright orange pigment. Deletion of the PKS gene cluster results in loss of the ability to activate the AhR receptor. AhR activation by L. reuteri has been shown to alleviate E. coli-induced mastitis in mice (Zhao C et al (2021) PLOS Pathogens 17(17):e1009774), and could be an effective approach against mastitis in other animals, particularly lactating animals, such as cattle for example. AhR activity and AhR-expressing micriobiota communications have been multi-factorially implicated, including in modulation of immune tolerance and response, intestinal homeostasis, carcinogenesis and intestinal barrier integrity (Dong F and Perdew G H (2020) Gut Microbes doi.org/10.1080/19490976.202.1859812). AhR has been implicated in various inflammatory- and immune-mediated conditions, such as atopic dermatitis.

Lactobacillus reuteri strain 3632 (ATCC PTA-126788) produces an orange pigment and includes a biosynthetic gene cluster (BGC) that encodes for a polyketide synthase which can act as an AhR-activating metabolite. The L. reuteri metabolite appears to give an orange pigmentation to the strain and is primarily associated with cell wall or cell envelope. Lactobacillus reuteri strain 3632 (ATCC PTA-126788) is described as having a characteristic orange pigment, including in Kumar et al, WO 2020/163398A1, published Aug. 13, 2020, the entire contents of which is incorporated herein by reference. Lactobacillus reuteri strain 3632 is detailed and described, including nucleic acid sequence in Kumar et al, WO 2020/163398A1, published Aug. 13, 2020, the entire contents of which is incorporated herein by reference. The genomic contigs and genome nucleic acid sequence of L. reuteri strain 3632 is also set out herein and provided in SEQ ID NOs: 49-55.

The stimulation of aryl hydrocarbon receptor (AhR) was evaluated with L. reuteri strain 3632-derived materials and with standard AhR ligands. 6-Formylindolo[3,2-b]carbazole (FICZ) was used as a standard AhR ligand compound, and its structure is depicted below:

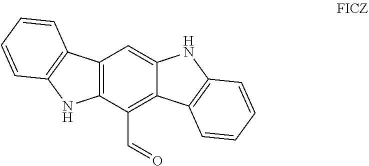 [SMILES: O=Cc1c2[nH]c3ccccc3c2cc2[nH]c3ccccc3c12]

Methods utilized to assess AhR activation in vitro were as follows: HepG2-Lucia™ AhR cells (Invivogen, hpgl-ahr) were grown at 37° C., 5% CO₂ in MEM (Thermo Fisher, 616965-026), 10% iFCS. For selection purposes, culture medium was supplemented with 100 μg/ml zeocin (Invivogen, ant-zn-5).

### Ligand Test Items:

FICZ: 6-Formylindolo[3,2-b]carbazole; AhR Agonist (Invivogen, tlrl-ficz)
L-Kynurenine: β-Anthraniloyl-L-Alanine; AhR Agonist (Invivogen, tlrl-kyn)
Purified L. reuteri PKS metabolite in a glass vial 2.5 mg/ml in DMSO was prepared as follows:
   (a) L. reuteri 3632 cell pellet was resuspended in 45 ml PBS (same volume as the corresponding culture supernatant), (for some samples: filtration via 0.2 μM)
   (b) L. reuteri 3632 supernatant, sterile filtered via 0.2 μM Media control, sterile filtered via 0.2 μM
The dilution of ligands was performed at 2× of the intended final concentration in 40 ml complete growth medium. +40 μl 2.2×10⁵ cells/ml
Culture supernatants were harvested at different time points (e. g. 48 h) and subjected to Luciferase Assay enzyme assays.

### Measurement of Luciferase in 384 Well, White Plate:

10 μl culture supernatant/well sample
+15 μl/well Quanti-Luc/Coelenterazine-utilizing luciferase detection medium (Invivogen rep-qlc) in 50 ml ddH2O (instead 25 ml). The reaction is monitored at room temperature (RT) with BioTek plate Reader.

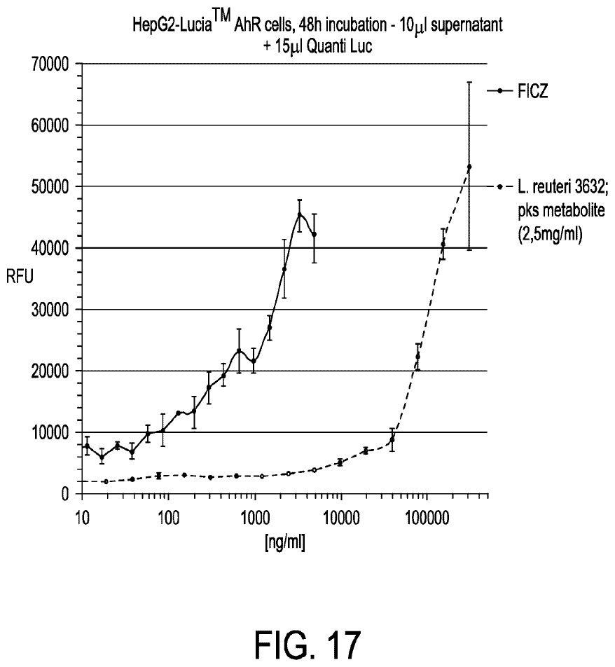

The first in vitro data from HepG2-Lucia cells demonstrated dose-dependent AHR activation by fractions from L. reuteri LR3632 extract enriched for pks metabolite. Initial data results are depicted in FIG. 17, which compares AhR activator activity of control FICZ versus L reuteri 3632 pks metabolite (2.5 mg/ml).

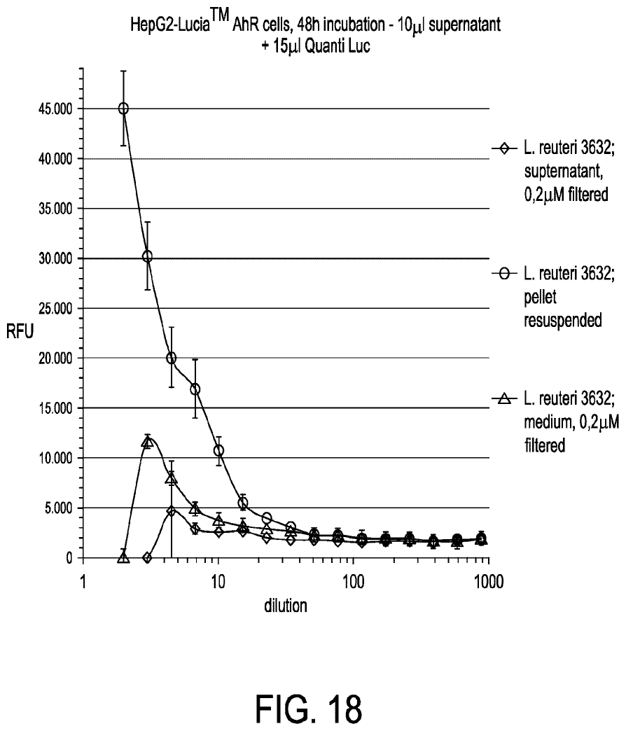

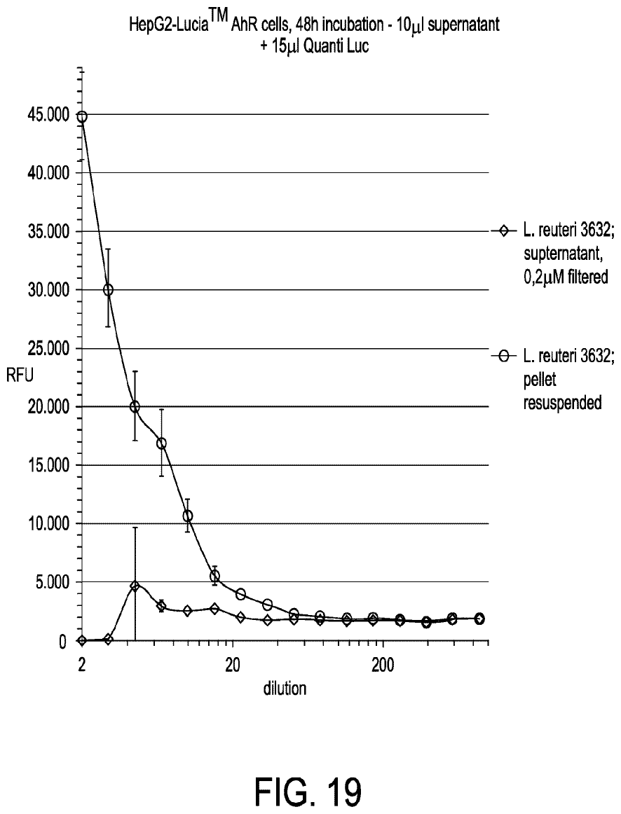

Additional AHR activation studies were conducted in accordance with the methods described above wherein AHR activator activity was assessed in L. reuteri 3632 supernatant, cell pellet resuspended, and medium (FIG. 18). Significant AhR activator activity was identified in the L reuteri 3632 pellet resuspended. The medium and supernatant had limited to no significant activity. To volume match the samples evaluated, the cell pellet was resuspended in the same volume as the culture supernatant and a direct comparison of ligand presence in the supernatant versus the pellet resusupended was conducted (FIG. 19). Again, significant Ahr activity is demonstrated in the pellet resuspended with little activity in the L reuteri strain 3632 0.2 μM filtered supernatant.

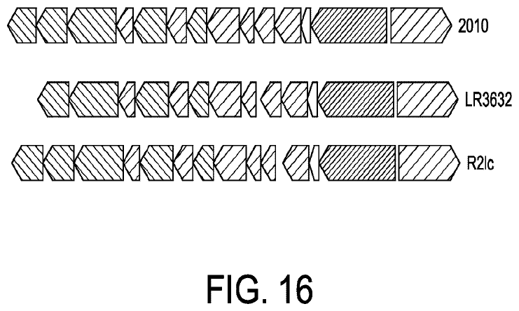

The PKS cluster from Lactobacillus reuteri which can produce the candidate AhR-activating metabolite has been assessed and determined from L. reuteri 3632. In particular, the Lactobacillus reuteri strain 3632 (ATCC PTA-126788) contains a BGC that encodes for a full suite of proteins required for synthesis and production of the AhR-activating metabolite. This L. reuteri 3632 cluster was compared to previously described PKS gene clusters encoding and producing Ahr metabolite from gut symbiont L reuteri strains R21c and 2010 (FIG. 16). Ozcam et al describes and assesses strains R2Ic and 2010 as encoding a polyketide synthase cluster that activates the mammaliam aryl hydrocarbon receptor (Ozcam M et al (2019) Applied and Environ Microbiology 85(1):e01661-18).

The PKS gene cluster from Lactobacillus reuteri strain 3632 is diagrammed and shown in FIG. 16. The BGC is encoded on a conjugation plasmid of 165 kb. The BGC contains 15 genes that encode for a full suite of proteins needed for the synthesis of AhR-activating metabolite (TABLE 16). Within the cluster, typical PKS modules are encoded for chain extension, reduction, and dehydration. A multi-subunit biotin-dependent acetyl-CoA carboxylase enzyme converts acetyl-CoA to malonate, which acts as the primary building block for the metabolite (pksK-N). An unusual feature of the BGC is the lack of a polyketide thioesterase enzyme that would release the full-length natural product from its acyl-carrier protein. Instead, a glyoxylase-like enzyme, pksG, likely serves this purpose. The gene cluster enables synthesis and production of active and effective AhR-activating metabolite by L. reuteri strain 3632. Cloning an additional copy of the cluster or introducing highly expressed constitutive or inducible promoters in the gene cluster can be utilized to increase the Ahr metabolite expression and production or to control or regulate Ahr activation by the L. reuteri strain.

TABLE 16
Genes and encoded proteins involved in the synthesis of Ahr-activating metabolite

| | | | Length | | |
|---|---|---|---|---|---|
| | | | (amino | | Amino acid |
| Locus tag | Gene | Description | acids) | Remarks | SEQ ID NO: |
| LREU3632_{—} | emrY/ | putative multidrug | 561 | Transporter | SEQ ID |
| 02405 | pksA | resistance protein EmrY | | | NO: 78 |
| LREU3632_{—} | fabF_{—} | 3-oxoacyl-[acyl-carrier- | 699 | | SEQ ID |
| 02406 | 2/pksB | protein] synthase 2 | | | NO: 79 |
| LREU3632_{—} | pksC | acyl carrier protein | 90 | | SEQ ID |
| 02407 | | | | | NO: 80 |
| LREU3632_{—} | fabG_{—} | 3-oxoacyl-[acyl-carrier- | 239 | reductase | SEQ ID |
| 02408 | 2/pksD | protein] reductase FabG | | | NO: 81 |
| LREU3632_{—} | pksE | [acyl-carrier-protein] | 175 | reductase | SEQ ID |
| 02409 | | reductase | | | NO: 82 |
| LREU3632_{—} | fabZ | 3-hydroxyacyl-[acyl- | 112 | | SEQ ID |
| 02410 | 3/pksF | carrier-protein] | | | NO: 83 |
| | | dehydratase FabZ | | | |
| LREU3632_{—} | pksG | Glyoxylase/metallo- | 298 | thioesterase | SEQ ID |
| 02411 | | beta-lactamase | | | NO: 84 |
| | | superfamily protein | | | |
| LREU3632_{—} | pksH | Transferase protein | 184 | transferase | SEQ ID |
| 02412 | | | | | NO: 85 |
| LREU3632_{—} | pksI | Transcriptional regulator | 177 | May not be | SEQ ID |
| 02413 | | PadR-like family protein | | required | NO: 86 |
| LREU3632_{—} | baeC/ | Polyketide biosynthesis | 317 | | SEQ ID |
| 02414 | pksJ | malonyl CoA-acyl | | | NO: 87 |
| | | carrierprotein | | | |
| | | transacylase BaeC | | | |
| LREU3632_{—} | accB/ | Biotin carboxyl carrier | 142 | | SEQ ID |
| 02415 | pksK | protein of acetyl-CoA | | | NO: 88 |
| | | carboxylase | | | |
| LREU3632_{—} | cfiB/ | 2-oxoglutarate | 459 | | SEQ ID |
| 02416 | pksL | carboxylase small | | | NO: 89 |
| | | subunit | | | |
| LREU3632_{—} | accD | Acetyl-coenzyme A | 279 | | SEQ ID |
| 02417 | 2/ | carboxylase carboxyl | | | NO: 90 |
| | pksM | transferase | | | |
| | | subunit beta | | | |
| LREU3632_{—} | accA | Acetyl-coenzyme A | 252 | | SEQ ID |
| 02261 | 2/ | carboxylase carboxyl | | | NO: 91 |
| | pksN | transferase subunit | | | |
| | | alpha | | | |
| LREU3632_{—} | pksO | bifunctional biotin-- | 144 | ligase | SEQ ID |
| 02262 | | [acetyl-CoA-carboxylase] | | | NO: 92 |
| | | synthetase/biotin operon | | | |
| | | repressor | | | |

Note that LREU3632_02265 indicated in Table 16 may not be required.

Strains are evaluated for AhR activity as follows. The strains are grown overnight in LB media. The cell pellet and the filter sterilized culture supernatant is evaluated for AhR activity using HepG2-Lucia™ AhR cells (Invivogen, hpgl-ahr). HepG2-Lucia™ AhR cells will be grown at 37° C., 5% CO₂ in MEM (Thermo Fisher, 616965-026), with 10% iFCS. For selection purposes, culture medium is supplemented with 100 μg/ml zeocin (Invivogen, ant-zn-5). FICZ (6-Formylindolo[3,2-b]carbazole; AhR Agonist and L-Kynurenine will be used as positive controls. Media control and strain controls are also included. The dilution of test material is performed at 2× of the intended final concentration in 40 ml complete growth medium and added with 40 μl of 2.2×10⁵ cells/ml. Culture supernatants are harvested at different time points (over 48 h) and subjected to Luciferase Assay. For measuring luciferase activity in 384 well, white plate, 10 μl culture supernatant/well sample is added with 15 μl/well of Quanti-Luc/Coelenterazine-utilizing luciferase detection medium (Invivogen rep-qlc) in 50 ml ddH2O (instead 25 ml). The reaction is monitored at room temperature with BioTek plate Reader.

Efficacy of the AhR metabolite in vivo is further assessed using a mouse atopic dermatitis (AD) animal model (Martel B C et al (2017) Yale J Biol and Med 90:389-402). The AhR activator tapinarof is in clinical development for treatment of psoriasis and atopic dermatitis (AD) in humans (Bissonnette R et al (2021) J Am Acad Dermatol 84:1059-1067; Lebwohl M et al (2020) Skin J Cutane Med 4(6):s75). Tapinarof is a secondary metabolite from Photorhabdus luminescens.

The full sequence of the L. reuteri strain 3632 PKS cluster along with 5 kb flanking regions on both sides of the BGC, corresponding in total to 12,969 bp is provided below (SEQ ID NO: 77).

```
        1 gcaagaattt atcccatagt ttgcgagaaa tccttttaga agagcttgcc taataatagc
      61 ggctcaatat aatttaaatt taaaaccacc taattagcaa tcgtaaaatt gttaattagg
    121 tggttttagt atagaaataa cccctaaatt cagaatacta gaaaatctag tactattatt
    181 ttttcctttt cgggaataat agacatgaaa gacaagaaaa gataagaaat ggtaaggaaa
    241 ctttgtacag tttaataaaa gcagttgagt attgactatt ggcgtaagtt ttgatttttt
    301 taattgtaat attaatagaa ttatttaatc tatgaaatac taatttcaca tttgcttttg
    361 tatgtgaata aatctctttt tcttggatct tagagggttc gtaaggtaag gaattaattt
    421 gtttagtata ttctttatcc gtagtttctt taattaattt cttcgaaaaa tgctttgatg
    481 gtgttcgaga gtttgaattg ttgtttaagc ttttaataga gttatctctt attttatttt
    541 tttgatattt aggaatattt aagttattga cctgctgatt gatgtaacta atagaattat
    601 gctttgcggt atttaaattc ccatatagtc cagttagaaa tattgctaca gctaaggaaa
    661 cccctacttg tcttaaaaca cctgcaacac tttgtgaagc ggttaagaga taaccttcaa
    721 agtttgatgc tgctaaaact gttattggac ctgcaataat cccataccca gttcctaaaa
    781 taatacaagt aaaaataatt aaggtaatat tatccatatc aatatgtgta aataaatagt
    841 aagagatacc cattaataga aaaccagata agataatggc tcgagatcct aatttgtcta
    901 agagaattgc agaaagtggt gacatgataa aaatcattcc tgtaataggc gtgataagaa
    961 gtgcagcttc taattcggtt ctatgttgta tatgggtaaa atatgttggt aaaattactg
  1021 taacagccac cagaaataaa ttacttaaaa ttatagaaac ggcagagccg gtaaattctc
  1081 tattcttaaa taaaacaaga ggaatcattg gtgctttagc ataatgctca gtaattaaga
  1141 atagtatgaa agttaaaatg aaaatgaaaa acagaaggtt gattgcagga cttttccacc
  1201 cccagcttct tccttgagtc aatattagag ttaaagctga aagtaaaatt atacctaaaa
  1261 gggaacctaa ataatcattt ccttcatttg actttggttc gtgaaagttg aaagtagata
  1321 aacaaataac tagtgaaagt atagtaaaag gtatattaac gctaaatata gaatgccagc
  1381 ccataaattg agttagaata cctccgattg aaggaccaag agcagcagct aaaccttgag
  1441 tgattcctaa tgcggcaata actttttttc ttgtctttac tgtaacgata tttattccaa
  1501 tagtcattga aagtggaaaa agaattgcgg ctcctaatga ttgaatgcca cggccaagaa
  1561 ttaaaatagc taggttagga cttataccag aaatacttga tccaataaga aacataatta
  1621 acccaattat atataagaaa ttcataccta atttttctgc aagtttagat agtgggatcg
  1681 ttaaactagc aaaaagtata gtatatatgt ttaatgccca agacaaatta gttagagtga
  1741 cgtgaagttc gttttgaata gctggcaatg caatattcat aacagttgta tctaacatac
  1801 ataagaaaat actaatacac attgcaatta ttattagaaa tttgtttttt cttttggtca
  1861 tacttgtttc ctcactttgt ttatattaac aatgttaata taaacaaaag aaaaataaat
  1921 gtcaacaata ttatcattgt aaatatgaac aatatgtatt aatataaata aaaattagtg
  1981 aggtgatttt atgacagctg taatagttgg aattggtatt actagttcct gtggagagtc
  2041 ctttacagaa attgaacaaa atgtaggtaa aggaaaaaca ggtatttcta atatcgatta
  2101 ttttgataca tctgagttaa catgtggaat tgctggtaac ttatcaaaga aaatatggaa
  2161 agaggttttg caaattgctg ataaaaatga attagattgg agtagtagtc tttcaattta
  2221 tactattcag cgattacttg aatcgtataa tatttcaaaa aaacaaagaa ttggtttatc
  2281 tcttggaaca tgtaatgggg gaattcattc tttagcagaa tatcttgata cttcaaatga
  2341 taaattttta aaaaattatc ctccgtacat tcagagtaaa gatatcgcac attatttcaa
  2401 ctttaacggg ccaaagtatt cttttaattc agcttgtgcg gctagtgcta atgcaatagc
  2461 ttatggagca gaaatgatta ataatagcga tgcagatctt gttgtgacgg gtggttgcga
  2521 tccaatgtca gaatgggttt ttgctgggtt taattcatta agaaccttta atagtaaaaa
  2581 ttgtatgcca tacggtgagg aatatggact taacctagga gaagctgcta catacttctt
  2641 attagaagat aaagataagg caattaaaaa agggcatagg atatatgcgg aaattttagg
  2701 gcacggctta tcaaatgatg cttatcatcc tacagcgcct gataaggatg gttcagggat
  2761 atcctatgca ataaaaatgg ctttaaaaaa ctcgggtctg aagcctgaag atattttata
  2821 tattaactct cacggaacag gaactaaagc caatgatagt gcagaataca gaggatttaa
  2881 aactgttttt agaaatgaga tgccttttat tagctcaatg aaaggatacg taggccataa
  2941 tctgggagca gctgctagta cagaattagc tataagttta atcggtatga atagtcagaa
  3001 agttttgtat cctaacttta atttaaccaa gtatagagaa gattgtaacg atgagcatat
  3061 cttaaaaaag ccttattcat tagatggata tgaagatatc aactttataa acaataatgc
  3121 tgcgtttggt ggacaaaatg ttgctgtaat tttccatgtt aatttagaag gaaaatatgg
  3181 tcatagtgaa aagaaactta aaacacagca acctatatat attaataatt ttggcgttgc
  3241 aagtgataaa gcttatatga caaagcatgg tattggtatt cttgacgatt tacgcccctt
  3301 aaagaaaaaa tatccaaaat tatataagcg acggatgaat atgttaactc aagtaagtat
  3361 tatcgcggct aaacaaacat tgcaggatca gtatagtaat tgtgggcttg tgtatggtac
  3421 gccttttggt agtctttcat caacactaaa atatgttgat tctattcaaa aatatgggtt
  3481 taaaaatgct agtggggcat attttccaga tttagttatt aattcaacga cggggcatat
  3541 ttgtcaagcg ctttcattga agagttatag ttcatctata agttcaggag gtgatgaaga
  3601 cttaagagcc ttaattattg cacataatgc gcttaataaa gggtacgctt caactatgtt
  3661 agttggagca ggccaagaag agacagaact gggcaataaa gtgttgaagc gtgaagttaa
  3721 taatcatgca acatttttaa gccttagtaa caaaaaaatg caagaaacaa tagcagaggt
  3781 tctatcttcc ggtgcaatgg gatttaaaaa taaaaaagag cttttgacca ttatacgatc
  3841 gaaaatagat gagaagttgg ctaatgatgc agatctaaaa gtgataattc aaaataatag
  3901 tgaaattacg aacgacgaat taataagtta tttcaagaat gataacaata tcagtataaa
  3961 ttctgataat tttgcggata gtaattttaa atcatttgtt aatcatagaa atgaaaatag
  4021 gttgttgctt gttggaattt cacaagtcaa tgatgtttcg tttgcagata ttaaaaaaat
  4081 aaaatagaga aagaggtaat actgatgaag catagtattg aagaaattaa ggatatttta
  4141 aaagaaaaag tattaattga acgattagag ttagatgatg tagaacccaa tgatatttca
  4201 gataatgaaa atttatttga tgaagaagga ctagcgttag attccgtgga agcattagat
  4261 atcatgacag gaattagtga agaatttggc attgacacat caatgttagg gcaggaagat
  4321 ataaaccatt ttcaaagcgt taatgatatg gctaaatata tctcagagaa tgaatagagg
  4381 atagttatga aatcagtact tatcacggga ataactggag gaataggaag aaaattaact
  4441 gaagcttatt cttcaaaggg atatcatatt tatggtacgt gtagtagaaa ttcagactct
  4501 ttacaacaat ttaaagagaa atggcctagt gttgaaataa ttcaaataaa tcatgatgac
  4561 ttgatagatg taagtacaga atattccttt ttttttagaa aagtgcaacc agatattgtt
  4621 attaacaatg ctggaatagt gaaagataat tttttagttc aaatgtcagt gaatgatttt
  4681 caagaagtgt tgactactaa tttgatttcg gcttgggtaa tagtcaaaga aatgctttta
  4741 catttaaatg ataataaaat tcataaaatc attaatgtag cttcaatatc tggaattata
  4801 gggcgtgaag gacagtgtaa ctatgcagca actaagggag gattagtagg cttatgtcaa
  4861 ttgatagaac atttagctcc gaaaggcagt aatgttattt cattttcagt tgcaccagga
  4921 ttaattgata cagatataaa gggaaaaatg ccaaagaaaa aaatcgataa tttaaagaag
  4981 gccacattgg caaataggct aggaacacca gaggaagtat caaaatttat ttttaaacta
  5041 tctgaagagg atatttcgta tagtgatgga actttatata ggattgatgg tggagtttta
  5101 aaatgaagat aataaataat acgattcaag taaccgactt gcttcaatca tatggaaaag
  5161 atgaaaaaga catagtaata attggtccca gcccatttaa tgaattagat tgccttaaag
  5221 aaacaaaaat aatcgataaa gttcagctta atttagagga agttttttca tttgtaaaaa
  5281 ataatagcgt tgctttaatg aaaaaaagaa gaggaaccat tgccttttta ttaaatccac
  5341 aaagttttga gggaggcaat aatatctatt ctccaatcta taattcagca attaagagct
  5401 ttttgaaatc cttatcgaag gaaatgaatc cattcagagt taaagttatg gggataatct
  5461 tacctttaac acaagataca aaatcgactc ggaaatatga tttagttaca ttaaagtaca
  5521 aaggaattaa taatgaaaag caggtacaag atattctaag cttacttaag ctttcagaaa
  5581 tactaaacgg acaaattgtc tcgctaggcg ctgaattgaa tctttagtaa aggagaggga
  5641 aatcatttta tttctagatc aaaatgcagt tataaaaatg ttgccgcaga aggagccatt
  5701 caggttctta gatacggttg aatgctttga tagagaaaaa cgtatgatta ctgcattgca
  5761 acaatttggt aatgaggaat tcttctttaa gggtcatttt ccaaataatc caatagttcc
  5821 aggggtactt ctaacagaat cgattgctca agcaggtttg atattgattt ctttattaga
  5881 aggccaaaaa gtgaaaattg gatatctagc ccaaattgaa aagacaaaat ttttcaaaga
  5941 ggtctatccc gacgagcaag taaaggttaa atgttcatta aagaaaaaaa taggtaaata
  6001 ctattacatt gcaggagaag tctactcgca gcaattaaat aaaagatgta tgagagcaac
  6061 agtaatagta tgtatttgat atggtgatgg ttcatgataa aaattactga aaatatagtt
  6121 caaataaaac taaagcaagg caaaaattat ccagacgtta atgtttatgt acttctaaaa
  6181 gaaaaagtgt taattgatat aggccccaaa tcaattaata cacttaacct tttaaaaaaa
  6241 gaattagcta ggttaggatt aagttttgaa acacttaatt taattattct aactcaccat
  6301 catgttgacc atgtaggact tttagagtac cttccctctg ggttacgtat cgttggacct
  6361 gatcatttag acttttatag ttcagatatt tataaaaaaa gtattcaaaa attgttagtt
  6421 gacgataatc tctctattga atttaaaaat gatatagaaa aacaacttac tactgaaata
  6481 attccaagta ttaatagaga aaactacgtt ccttttagtg agtcaaagaa aattttgcaa
  6541 caatttggtc ttacagctgt agagctatca ggtcattcta gtgaagatat tgtaattact
  6601 gattcagaaa ataactgttt tactggtgat attattattc ctaaaatttt ttttaactgt
  6661 atatatgagg ttgataaagt aaggcccaaa catcaacgtt ggtcatatta tcatgagctt
  6721 aattttttag acaggttagt aaacctagta ttgccaggac atggagatat tctaaagtta
  6781 gaagaattaa aaaaggcagt tttggttaat agaaaaagaa tgagacggac agagaaaaaa
  6841 ataataagag agttaaataa agaaacagtt aatggagtgg agaatgtttg tcgaagtgta
  6901 ttccaaagtt ttttgcctta tagcaaattc ttaccgtttt cagaagtagt tagtgttatc
  6961 gagagtaatg atgagagaat taactattga tattatttat ataaatacac taaataataa
  7021 cagctttgca agtttagatt ctagaaaaag aatcaaaaag ttacagcatc agctaggaca
  7081 atatatgctt tctcaaatat cttataaaaa aggttattca atttcccata gtcatatgtg
  7141 tgtagcctta gcaagttatg ttaatagggt tggtattgat attgaactta taaataaaac
  7201 aaagaaggca aggatacagt tcctttcgaa aagcgaaaaa caattagtaa atagatatgg
  7261 ttttacaaga atatggacac taaaggaagc tatagctaag tatcataccg ttgggttacc
  7321 tcggcttaat acagttgaga ttaaagaaat aaatgcttcg aacgttatct attttgttaa
  7381 taaagcacca agaaaattgc agtataaatt tttagatata atcccttctt ataggttgag
  7441 tgtagtggca aaaaaggtat caagtttttg cattagaatc acacaagaag aagatttgaa
  7501 ggggctaata aggagaatgt aattaatgca aggaagagac ataattctag gaatacttga
  7561 acgaaataat agaacaggat atgaaattaa tgatattttg aaaaatcaat tatcttattt
  7621 ctatgacgga acatatggaa tgatatatcc aacattacga aagcttgaaa aggaaggaaa
  7681 aataaaaaag gaaaaaattg tgcaaaatga taagccaaat aagaatgttt attctattac
  7741 agatacggga atagaagaat ttaaagaata cttggattct tctattcaag atgatattta
  7801 taaatcggac tttttaatgc gtctcttttt tggaaactca ttaccaaatc aagaaattat
  7861 tagagctatt aaacaagaga tacaacgtaa aaatgaaaag atagatcaat taactgtaaa
  7921 ttataaaaaa tggaaaaaaa atggaatgag taaaacacaa gaaataaccg tcaaatatgg
  7981 tattgcgcag tataccgcaa ttgttcagat gctgacaaaa gagcttgata cgttacgaag
  8041 aaatgagatg gatgattaat gtcattagga attatattta gtgggcaagg agcacaaaaa
  8101 tctaaaatgg gtcttgattt ttatgaagat ccactatttg ctgaattact taaccacgca
  8161 agtaatatat ctggtctaaa catgttaaaa atcttggaaa acaaaaataa tgagcttaca
  8221 gagactgtta atttacaacc aacattaaca acattgaatt atggcatata tcgaatgctg
  8281 aaaagggata tttttgatat gaaagtaagc tgtatggcag ggctttcttt aggagaatat
  8341 tctgcactga ttgcttctaa tgctttaact tttgaacagg gaatacaatt actagtagac
  8401 cgcggaaaat atatgcagga agcttcgaat agtaatgcag gaaaaatgtt agcacttata
  8461 aaacctaaac taaaagagat aactcaaatt tgtgctttgt gtaaggttga aattgctaat
  8521 tataattctc caaagcagat agtaataggc ggacaaaatt tgcaaattga atttgcaaag
  8581 aaaatgatta tggagcgtaa agctgcatta agaataattg agttagaagt aagtggcgcc
  8641 tttcatactt cgttattttc aaatgttcaa aaacaattgg aaaagcgatt aaaagatgtt
  8701 aaatttgaga atccacaaat tccggtagtt agtaatacta cagttgagga atttcaaaag
  8761 gaaagtctta cagccgtatt atcaaaacaa gttgctaatc ctacatattt tgaaaaagat
  8821 attaagttaa tgaaaaatac ttatggattg acacatatag ttcaaattgg tcctggtaag
  8881 gcattaagta attttgtgaa gcaaatgtca ttaggaatta agacatataa tatttctaat
  8941 ataaaagact atagaaaatt tctgaatagt tatagagata ttaatttgaa aggaaagaaa
  9001 aatggatttt gagaaaattc agcaactaat tcaaatgttt gaaagctcta atacaagaga
  9061 actaaaaata gatgataata actttcacat ttatcttaca aaaaacgtaa gtaaagaacc
  9121 aattcacgat ataaaatttg aatcgaataa aattcagcaa gcctcagagg caaaagcaaa
  9181 caggaaaacg ataacagccc cattggttgg aacagtttat ttagcctctt ctcctacgtc
  9241 taaaccgtat gttcaagtag ggagccatat tgataaagga gatacagtgt gtgtaataga
  9301 ggctatgaag ctaatgacgg aaataaaaag tgaggttacc ggaacaattg aaaaagttaa
  9361 tgtagagaat ggagaattag ttgaagttgg gcaaccgtta ttttcagttt ctggagaaaa
  9421 agaaagttaa tgaaagagtg aggtgtagtg atgttttcta aagtactagt tgctaatcgt
  9481 ggagaaatag ctgttcgtat tattagaact cttcatgaat tgggcattaa agcggtagct
  9541 atttattcga ttgttgatca agaaagtctt catgttcaat tggctgatga agcagtttgt
  9601 gtaggtggag cacgaccaca ggattcgtat ttaaatacta ctaatatttt aacagcagca
  9661 attggaacag gagcacaagc gatacaccct ggctttggat tcctttcaga aaacgctgag
  9721 tttgcaagaa tgtgtgaaaa atgcggaata gtatttatag gtccgcgtgc tgctacgatt
  9781 gatttaatgg gaaataaaga aaatgcgcgc gagactatgc aaaagagagg gatacctgtt
  9841 attcctggaa gtagttccta cataacaaac agttatgatg caaaaagagt tgcagataaa
  9901 ataggatatc ctattttaat aaaggcggca gctgggggtg gaggtaaagg tattagaaga
  9961 gttgtaacgc ctgagcaaat gaaacaagaa tttaataatg cgcaaagaga agctcgtata
10021 tcatttggtg atgatcgtat gtaccttgaa aaaataatgt gcaatgttaa acatattgaa
10081 gtacaggtag ttcgtgatag attcggaaat agtgtctatt ttccagaacg cgattgttca
10141 ctccaacggg ataagcaaaa aattatagaa gagagtccat gctcggttat caattcagaa
10201 caaagaaaaa ttttaggaag gtatgctatt aaagcaattg aagcagttga ttatcttaat
10261 acaggaacta ttgaattttt gatggataaa aataataaat tttattttat ggaaatgaat
10321 actcgtattc aagttgaaca tactgtaact gagatggtaa cgggaataga tctagtaaag
10381 gtacaactga tgatcgcttc aggtgaggaa cttccttttt cacaaaataa tattaagtta
10441 aatggggtag ctattgagtg tagaattaat gcagaagatc ctaaaaataa ctttgttcct
10501 tcaactggaa aaataaatta tttatattta cctgtaggta acttgggtat gagaattgat
10561 actgcacttt actctggaga aaagataaca ccattttatg attcaatgat tgcaaaagta
10621 atctcgcatg gccatactcg cgtagaagcg atcaatagaa tgaagaggtt aatgcaagaa
10681 ctggttataa aaggtgttaa aacaaataaa gatcttcact tatcaatttt gggagattca
10741 agttttttga aagatacagt aacaactgag tatttagaga aaaacttttt accaatctgg
10801 aaagagaggg agaaaaatgc agctgtatga aagtgcaacg ttaactaagc aacatgtaaa
10861 ggcgaatatt aaagcaaatc aaaaagtacc tgatgggatg ttaaaaaaat gtcctaagtg
10921 tgggaaaatc tttctgtcta cagaatttga taagtatttt tcgtgtcctg gttgtaatta
10981 tggatttagg attggttcat ggcaaagggt agcctgggcg gttgatgaat tttatgaaga
11041 ggatggttca cgagaactaa taacctcaga tcctttacat tttcctaatt atgtaaaaaa
11101 gattcaagga cttcaaaaga ctactaaggt aaatgaagca gttttaacgg gtaaagctaa
11161 aataaaagac caaatttttg agtgtggcat tatggatcct cactttataa tgggatcatt
11221 aggaacaatt actggcgaaa agattacgag attatttgaa cgtgctacaa aggagaggtt
11281 gcctgttgtc ctatggactg cttcaggggg agctagaatg caagaaggaa ttatgtcatt
11341 aatgcagatg gcaaaaataa gtcaagctat agcacaacat gcatcgaaag gacttttata
11401 cattgttatt ttaactgatc caacaacagg tggagtaaca gcaagttttg caatgcaagg
11461 agacattatt ttagcagagc cacatacttt agtaggattt gcagggcggc gagttattga
11521 gcaaactatt catgagagaa tcccagatac gttacaggat gcggagaatg taataaaaca
11581 tggattcata gatttaatag tatctcggag tgaagaaaaa tctttacttt ataaattgtt
11641 aaaatatggt aggtgataat aatggcaact gagaatgcaa tggcaattgt aaaagctgct
11701 cgaagtgata ataaaataac tgctaaagaa attatagaag aagtattttc agattttatt
11761 gaattccatg gtgatcgtaa aggaacagat gattcagcaa ttcttggagg attggcaata
11821 ctttcatcta caccagtaac agtaattgcg actaataggg gggaaacagt tggtgaacat
11881 ttgagtacac attttgggtg tccaactcca ggtggctatc gtaaagcttt gcgtttagca
11941 aagcaggctg caaagtttaa tagaccaatt atatttttag taaatactcc tggagcttat
12001 ccagggaaaa cagctgaaga acaagggcaa ggatctgcga ttgcgcaaaa tattatccaa
12061 ataagtcaac ttccagttcc aattattact attatttatg gtgaaggtgg cagtggaggt
12121 gcattagcat tggcttgcgg tgatcaggtg tggatgctag agaatagtac atattcaatt
12181 ttatcacctg agggttttgc ctctatttta tggaaagatg gatcacgtac agaagaagcc
12241 gcagaattaa tgcaaatgac cccaaaagcc ttgctaagac ataaggttat tgaaggaata
12301 attccagaaa gtaaagatca caaagtaaca tgtcgagcaa ttgctaatat tttagataaa
12361 cagttacaag aattaggaaa actttcagcg gtagatttat tgattcaacg aaaaaaaga
12421 tatagaaaat tttagttagt atagtaagag gagagaatac attacggtgc acgacttatt
12481 atcgcttaat agaattaaag aattatataa agagaaagca atgacaaatt gcaacttttt
12541 ttactttaat aaagtagatt ctactcaaaa aatagcaaaa tattttttag accatttaaa
12601 tatcaataaa ccatgtacta tttttgctgg aagtcaaacg agaggctatg gtaaaagaaa
12661 taggctattt tattcaccac aaaattcggg gatttatatg agtataatta ttccacaata
12721 cgaaatgcaa aatgaggata ttggtttatt aacaatttca ttagggagtg gaatactaga
12781 agttttaaag aagtattatc ctaaaaaaat tttttattta aaatgggtaa atgatattct
12841 attaggcaag gtgaactact cggccataaa tgaccgagct tctgggaaca cgaagtagta
12901 tttaggatat tacacaagta ttcttgctac ctaacttaca gaactccgtt cttttaccaa
12961 ggctagttc
```

The amino acid sequences for the PKS gene cluster proteins noted in Table 16 and encoded by the gene cluster are provided below. Annotations providing their locations in the 12,969 bp cluster sequence referenced above and corresponding to genome map locations are also indicated.

| LOCUS | PKS_encoding_region 12969 bp DNA |
|---|---|
| DEFINITION | Lactobacillus reuteri strain 3632. |
| SOURCE | Lactobacillus reuteri |
| ORGANISM | Lactobacillus reuteri |
| FEATURES | Location/Qualifiers |
| source | join(11662 . . . >12969, <1 . . . 11661) |
| | /organism = “Lactobacillus reuteri” |
| | /mol_type = “genomic DNA” |
| | /strain = 3632 |
| CDS | complement(176 . . . 1861) |
| | /gene = “emrY” |
| | /locus_tag = “LREU3632_02405” |
| | /inference = “ab initio prediction:Prodigal:2.6” |
| | /inference = “similar to AA sequence:UniProtKB:P52600” |
| | /product = “putative multidrug resistance protein EmrY” |
| | /translation = “MTKRKNKFLIIIAMCISIFLCMLDTTVMNIALPAIQNELHVTLT |
| | NLSWALNIYTILFASLTIPLSKLAEKLGMNFLYIIGLIMFLIGSSISGISPNLAILIL |
| | GRGIQSLGAAILFPLSMTIGINIVTVKTRKKVIAALGITQGLAAALGPSIGGILTQFM |
| | GWHSIFSVNIPFTILSLVICLSTFNFHEPKSNEGNDYLGSLLGIILLSALTLILTQGR |
| | SWGWKSPAINLLFFIFILTFILFLITEHYAKAPMIPLVLFKNREFTGSAVSIILSNLF |
| | LVAVTVILPTYFTHIQHRTELEAALLITPITGMIFIMSPLSAILLDKLGSRAIILSGF |
| | LLMGISYYLFTHIDMDNITLIIFTCIILGTGYGIIAGPITVLAASNFEGYLLTASQSV |
| | AGVLRQVGVSLAVAIFLTGLYGNLNTAKHNSISYINQQVNNLNIPKYQKNKIRDNSIK |
| | SLNNNSNSRTPSKHFSKKLIKETTDKEYTKQINSLPYEPSKIQEKEIYSHTKANVKLV |
| | FHRLNNSINITIKKIKTYANSQYSTAFIKLYKVSLPFLIFSCLSCLLFPKRKK” (SEQ ID NO: 78) |
| | /label = “emrY CDS” |
| CDS | 1991 . . . 4087 |
| | /gene = “fabF_2” |
| | /locus_tag = “LREU3632_02406” |
| | /EC_number = “2.3.1.179” |
| | /inference = “ab initio prediction:Prodigal:2.6” |
| | /inference = “similar to AA sequence:UniProtKB:Q9KQH9” |
| | /product = “3-oxoacyl-[acyl-carrier-protein] synthase 2” |
| | /translation = “MTAVIVGIGITSSCGESFTEIEQNVGKGKTGISNIDYFDTSELT |
| | CGIAGNLSKKIWKEVLQIADKNELDWSSSLSIYTIQRLLESYNISKKQRIGLSLGTCN |
| | GGIHSLAEYLDTSNDKFLKNYPPYIQSKDIAHYFNFNGPKYSFNSACAASANAIAYGA |
| | EMINNSDADLVVTGGCDPMSEWVFAGFNSLRTFNSKNCMPYGEEYGLNLGEAATYFLL |
| | EDKDKAIKKGHRIYAEILGHGLSNDAYHPTAPDKDGSGISYAIKMALKNSGLKPEDIL |
| | YINSHGTGTKANDSAEYRGFKTVFRNEMPFISSMKGYVGHNLGAAASTELAISLIGMN |
| | SQKVLYPNFNLTKYREDCNDEHILKKPYSLDGYEDINFINNNAAFGGQNVAVIFHVNL |
| | EGKYGHSEKKLKTQQPIYINNFGVASDKAYMTKHGIGILDDLRPLKKKYPKLYKRRMN |
| | MLTQVSIIAAKQTLQDQYSNCGLVYGTPFGSLSSTLKYVDSIQKYGFKNASGAYFPDL |
| | VINSTTGHICQALSLKSYSSSISSGGDEDLRALIIAHNALNKGYASTMLVGAGQEETE |
| | LGNKVLKREVNNHATFLSLSNKKMQETIAEVLSSGAMGFKNKKELLTIIRSKIDEKLA |
| | NDADLKVIIQNNSEITNDELISYFKNDNNISINSDNFADSNFKSFVNHRNENRLLLVG |
| | ISQVNDVSFADIKKIK” (SEQ ID NO: 79) |
| | /label = “fabF 2 CDS” |
| CDS | 4105 . . . 4377 |
| | /locus_tag = “LREU3632_02407” |
| | /inference = “ab initio prediction:Prodigal:2.6” |
| | /inference = “protein motif:CLUSTERS:PRK09184” |
| | /product = “acyl carrier protein” |
| | /translation = “MKHSIEEIKDILKEKVLIERLELDDVEPNDISDNENLFDEEGLA |
| | LDSVEALDIMTGISEEFGIDTSMLGQEDINHFQSVNDMAKYISENE” (SEQ ID NO: 80) |
| | /label = “acyl carrier protein CDS” |
| CDS | 4387 . . . 5106 |
| | /gene = “fabG_2” |
| | /locus_tag = “LREU3632_02408” |
| | /EC_number = “1.1.1.100” |
| | /inference = “ab initio prediction:Prodigal:2.6” |
| | /inference = “similar to AA sequence:UniProtKB:067610” |
| | /product = “3-oxoacyl-[acyl-carrier-protein] reductase FabG” |
| | /translation = “MKSVLITGITGGIGRKLTEAYSSKGYHIYGTCSRNSDSLQQFKE |
| | KWPSVEIIQINHDDLIDVSTEYSFFFRKVQPDIVINNAGIVKDNFLVQMSVNDFQEVL |
| | TTNLISAWVIVKEMLLHLNDNKIHKIINVASISGIIGREGQCNYAATKGGLVGLCQLI |
| | EHLAPKGSNVISFSVAPGLIDTDIKGKMPKKKIDNLKKATLANRLGTPEEVSKFIFKL |
| | SEEDISYSDGTLYRIDGGVLK” (SEQ ID NO: 81) |
| | /label = “fabG 2 CDS” |
| CDS | 5103 . . . 5627 |
| | /locus_tag = “LREU3632_02409” |
| | /inference = “ab initio prediction:Prodigal:2.6” |
| | /product = “hypothetical protein” |
| | /translation = “MKIINNTIQVTDLLQSYGKDEKDIVIIGPSPFNELDCLKETKII |
| | DKVQLNLEEVFSFVKNNSVALMKKRRGTIAFLLNPQSFEGGNNIYSPIYNSAIKSFLK |
| | SLSKEMNPFRVKVMGIILPLTQDTKSTRKYDLVTLKYKGINNEKQVQDILSLLKLSEI |
| | LNGQIVSLGAELNL” (SEQ ID NO: 82) |
| | /label = “hypothetical protein CDS” |
| CDS | 5744 . . . 6079 |
| | /gene = “fabZ_3” |
| | /locus_tag = “LREU3632_02410” |
| | /EC_number = “4.2.1.59” |
| | /inference = “ab initio prediction:Prodigal:2.6” |
| | /inference = “similar to AA sequence:UniProtKB:P64107” |
| | /product = “3-hydroxyacyl-[acyl-carrier-protein] dehydratase FabZ” |
| | /translation = “MITALQQFGNEEFFFKGHFPNNPIVPGVLLTESIAQAGLILISL |
| | LEGQKVKIGYLAQIEKTKFFKEVYPDEQVKVKCSLKKKIGKYYYIAGEVYSQQLNKRC |
| | MRATVIVCI” (SEQ ID NO: 83) |
| | /label = “fabZ 3 CDS” |
| CDS | 6094 . . . 6990 |
| | /locus_tag = “LREU3632_02411” |
| | /inference = “ab initio prediction:Prodigal:2.6” |
| | /inference = “protein motif:Pfam:PF00753.21” |
| | /product = “Metallo-beta-lactamase superfamily protein” |
| | /translation = “MIKITENIVQIKLKQGKNYPDVNVYVLLKEKVLIDIGPKSINTL |
| | NLLKKELARLGLSFETLNLIILTHHHVDHVGLLEYLPSGLRIVGPDHLDFYSSDIYKK |
| | SIQKLLVDDNLSIEFKNDIEKQLTTEIIPSINRENYVPFSESKKILQQFGLTAVELSG |
| | HSSEDIVITDSENNCFTGDIIIPKIFFNCIYEVDKVRPKHQRWSYYHELNFLDRLVNL |
| | VLPGHGDILKLEELKKAVLVNRKRMRRTEKKIIRELNKETVNGVENVCRSVFQSFLPY |
| | SKFLPFSEVVSVIESNDERINY” (SEQ ID NO: 84) |
| | /label = “Metallo-beta-lactamase superfamily protein CDS” |
| CDS | 6971 . . . 7522 |
| | /locus_tag = “LREU3632_02412” |
| | /inference = “ab initio prediction:Prodigal:2.6” |
| | /product = “hypothetical protein” |
| | /translation = “MRELTIDIIYINTLNNNSFASLDSRKRIKKLQHQLGQYMLSQIS |
| | YKKGYSISHSHMCVALASYVNRVGIDIELINKTKKARIQFLSKSEKQLVNRYGFTRIW |
| | TLKEAIAKYHTVGLPRLNTVEIKEINASNVIYFVNKAPRKLQYKFLDIIPSYRLSVVA |
| | KKVSSFCIRITQEEDLKGLIRRM” (SEQ ID NO: 85) |
| | /label = “hypothetical protein CDS” |
| CDS | 7526 . . . 8059 |
| | /locus_tag = “LREU3632_02413” |
| | /inference = “ab initio prediction:Prodigal:2.6” |
| | /inference = “protein motif:Pfam:PF03551.8” |
| | /product = “Transcriptional regulator PadR-like family protein” |
| | /translation = “MQGRDIILGILERNNRTGYEINDILKNQLSYFYDGTYGMIYPTL |
| | RKLEKEGKIKKEKIVQNDKPNKNVYSITDTGIEEFKEYLDSSIQDDIYKSDFLMRLFF |
| | GNSLPNQEIIRAIKQEIQRKNEKIDQLTVNYKKWKKNGMSKTQEITVKYGIAQYTAIV |
| | QMLTKELDTLRRNEMDD” (SEQ ID NO: 86) |
| | /label = “Transcriptional regulator PadR-like family protein |
| | CDS” |
| CDS | 8059 . . . 9012 |
| | /gene = “baeC” |
| | /locus_tag = “LREU3632_02414” |
| | /EC_number = “2.3.1.39” |
| | /inference = “ab initio prediction:Prodigal:2.6” |
| | /inference = “similar to AA sequence:UniProtKB:A7Z4X8” |
| | /product = “Polyketide biosynthesis malonyl CoA-acyl carrier protein |
| | transacylase BaeC” |
| | /translation = “MSLGIIFSGQGAQKSKMGLDFYEDPLFAELLNHASNISGLNMLK |
| | ILENKNNELTETVNLQPTLTTLNYGIYRMLKRDIFDMKVSCMAGLSLGEYSALIASNA |
| | LTFEQGIQLLVDRGKYMQEASNSNAGKMLALIKPKLKEITQICALCKVEIANYNSPKQ |
| | IVIGGQNLQIEFAKKMIMERKAALRIIELEVSGAFHTSLFSNVQKQLEKRLKDVKFEN |
| | PQIPVVSNTTVEEFQKESLTAVLSKQVANPTYFEKDIKLMKNTYGLTHIVQIGPGKAL |
| | SNFVKQMSLGIKTYNISNIKDYRKFLNSYRDINLKGKKNGF” (SEQ ID NO: 87) |
| | /label = “baeC CDS” |
| CDS | 9002 . . . 9430 |
| | /gene = “accB_2” |
| | /locus_tag = “LREU3632_02415” |
| | /inference = “ab initio prediction:Prodigal:2.6” |
| | /inference = “similar to AA sequence:UniProtKB:P0ABD8” |
| | /product = “Biotin carboxyl carrier protein of acetyl-CoA carboxylase” |
| | /translation = “MDFEKIQQLIQMFESSNTRELKIDDNNFHIYLTKNVSKEPIHDI |
| | KFESNKIQQASEAKANRKTITAPLVGTVYLASSPTSKPYVQVGSHIDKGDTVCVIEAM |
| | KLMTEIKSEVTGTIEKVNVENGELVEVGQPLFSVSGEKES” (SEQ ID NO: 88) |
| | /label = “accB 2 CDS” |
| CDS | 9451 . . . 10830 |
| | /gene = “cfiB_2” |
| | /locus_tag = “LREU3632_02416” |
| | /EC_number = “6.4.1.7” |
| | /inference = “ab initio prediction:Prodigal:2.6” |
| | /inference = “similar to AA sequence:UniProtKB:D3DJ42” |
| | /product = “2-oxoglutarate carboxylase small subunit” |
| | /translation = “MFSKVLVANRGEIAVRIIRTLHELGIKAVAIYSIVDQESLHVQL |
| | ADEAVCVGGARPQDSYLNTTNILTAAIGTGAQAIHPGFGFLSENAEFARMCEKCGIVF |
| | IGPRAATIDLMGNKENARETMQKRGIPVIPGSSSYITNSYDAKRVADKIGYPILIKAA |
| | AGGGGKGIRRVVTPEQMKQEFNNAQREARISFGDDRMYLEKIMCNVKHIEVQVVRDRF |
| | GNSVYFPERDCSLQRDKQKIIEESPCSVINSEQRKILGRYAIKAIEAVDYLNTGTIEF |
| | LMDKNNKFYFMEMNTRIQVEHTVTEMVTGIDLVKVQLMIASGEELPFSQNNIKLNGVA |
| | IECRINAEDPKNNFVPSTGKINYLYLPVGNLGMRIDTALYSGEKITPFYDSMIAKVIS |
| | HGHTRVEAINRMKRLMQELVIKGVKTNKDLHLSILGDSSFLKDTVTTEYLEKNFLPIW |
| | KEREKNAAV” (SEQ ID NO: 89) |
| | /label = “cfiB 2 CDS” |
| CDS | 10817 . . . 11656 |
| | /gene = “accD_2” |
| | /locus_tag = “LREU3632_02417” |
| | /EC_number = “6.4.1.2” |
| | /inference = “ab initio prediction:Prodigal:2.6” |
| | /inference = “similar to AA sequence:UniProtKB:P0CC08” |
| | /product = “Acetyl-coenzyme A carboxylase carboxyl transferase subunit beta” |
| | /translation = “MQLYESATLTKQHVKANIKANQKVPDGMLKKCPKCGKIFLSTEF |
| | DKYFSCPGCNYGFRIGSWQRVAWAVDEFYEEDGSRELITSDPLHFPNYVKKIQGLQKT |
| | TKVNEAVLTGKAKIKDQIFECGIMDPHFIMGSLGTITGEKITRLFERATKERLPVVLW |
| | TASGGARMQEGIMSLMQMAKISQAIAQHASKGLLYIVILTDPTTGGVTASFAMQGDII |
| | LAEPHTLVGFAGRRVIEQTIHERIPDTLQDAENVIKHGFIDLIVSRSEEKSLLYKLLK |
| | YGR” (SEQ ID NO: 90) |
| | /label = “accD 2 CDS” |
| CDS | 11680 . . . 12435 |
| | /gene = “accA_2” |
| | /locus_tag = “LREU3632_02261” |
| | /EC_number = “6.4.1.2” |
| | /inference = “ab initio prediction:Prodigal:2.6” |
| | /inference = “similar to AA sequence:UniProtKB:Q9FBB7” |
| | /product = “Acetyl-coenzyme A carboxylase carboxyl transferase subunit alpha” |
| | /translation = “MAIVKAARSDNKITAKEIIEEVFSDFIEFHGDRKGTDDSAILGG |
| | LAILSSTPVTVIATNRGETVGEHLSTHFGCPTPGGYRKALRLAKQAAKFNRPIIFLVN |
| | TPGAYPGKTAEEQGQGSAIAQNIIQISQLPVPIITIIYGEGGSGGALALACGDQVWML |
| | ENSTYSILSPEGFASILWKDGSRTEEAAELMQMTPKALLRHKVIEGIIPESKDHKVTC |
| | RAIANILDKQLQELGKLSAVDLLIQRKERYRKF” (SEQ ID NO: 91) |
| | /label = “accA 2 CDS” |
| CDS | 12467 . . . 12898 |
| | /locus_tag = “LREU3632_02262” |
| | /inference = “ab initio prediction:Prodigal:2.6” |
| | /inference = “protein motif:CLUSTERS:PRK11886” |
| | /product = “bifunctional biotin--[acetyl-CoA-carboxylase] |
| | synthetase/biotin operon repressor” |
| | /translation = “MHDLLSLNRIKELYKEKAMTNCNFFYFNKVDSTQKIAKYFLDHL |
| | NINKPCTIFAGSQTRGYGKRNRLFYSPQNSGIYMSIIIPQYEMQNEDIGLLTISLGSG |
| | ILEVLKKYYPKKIFYLKWVNDILLGKVNYSAINDRASGNTK” (SEQ ID NO: 92) |
| | /label = “bifunctional biotin--[acetyl-CoA-carboxylase] |
| | synthetase/biotin operon repressor CDS” |

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. (canceled)

2. (canceled)

3. (canceled)

4. (canceled)

5. (canceled)

6. (canceled)

7. (canceled)

8. (canceled)

9. (canceled)

10. A method for reducing intestinal permeability, alleviating alcohol induced or disease related leaky gut syndrome, alleviating the intestinal and systemic effects of chronic alcohol consumption, including intestinal dysbiosis, and/or reducing inflammation, including intestinal-derived or intestinal-associated inflammation, comprising administering an effective amount of an immunogenic probiotic composition comprising at least one of an isolated first Lactobacillus reuteri strain and an isolated second Lactobacillus reuteri strain wherein:
(a) the isolated first Lactobacillus reuteri strain has a genomic nucleic acid sequence including at least one of SEQ ID NOs: 49-55, sequences having one or more nucleic acid sequence difference from the sequence of at least one of SEQ ID NOs: 49-55, sequences having at least one nucleic acid sequence difference from the sequence of at least one of SEQ ID NOs: 49-55 and further having at least 97%, at least 98%, at least 99% or at least 99.5% sequence identity with one or more of SEQ ID NOs: 49-55, sequences having at least 98%, at least 98.5%, at least 99%, or at least 99.5% sequence identity to SEQ ID NOs: 49-55, and/or
the isolated second Lactobacillus reuteri strain has a genomic nucleic acid sequence including at least one of SEQ ID NOs: 44-48, sequences having one or more nucleic acid sequence difference from the sequence of at least one of SEQ ID NOs: 44-48, sequences having at least one nucleic acid sequence difference from the sequence of at least one of SEQ ID NOs: 44-48 and further having at least 97%, at least 98%, at least 99% or at least 99.5% sequence identity with one or more of SEQ ID NOs: 44-48, sequences having at least 98%, at least 98.5%, at least 99%, or at least 99.5% sequence identity to SEQ ID NOs: 44-48; or
(b) the isolated first Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788, or is Lactobacillus reuteri strain 3632, which corresponds to ATCC Patent Deposit Number PTA-126788; and/or
the isolated second Lactobacillus reuteri strain comprises or has a genomic nucleic acid sequence corresponding to the genomic nucleic acid sequence of ATCC strain PTA-126787, or a variant thereof comprising or having a nucleic acid sequence at least 98%, at least 98.5%, at least 99%, or at least 99.5% sequence identity to the genomic nucleic acid sequence of ATCC strain PTA-126787, or is Lactobacillus reuteri strain 3630, which corresponds to ATCC Patent Deposit Number PTA-126787.

11. The A method of claim 10 wherein the production or expression of one or more proinflammatory cytokine and/or for increasing the production or expression of one or more anti-inflammatory cytokine in the intestine of an animal.

12. The method of claim 10, wherein an isolated first Lactobacillus reuteri strain and an isolated second Lactobacillus reuteri strain are administered.

13. The method of claim 11, wherein the production or expression of one or more proinflammatory cytokine selected from IL-6, TNF-α, IFN-γ and IL-1, including IL-1β, is decreased in the intestine or gastrointestinal system of an animal, and/or wherein the production or expression of one or more anti-inflammatory cytokine selected from IL-10 and IL-22 is increased in the intestine or gastrointestinal system of an animal.

14. (canceled)

15. (canceled)

16. (canceled)

17. (canceled)

18. The method of claim 10, wherein the intestinal-derived or intestinal-associated inflammation is associated with gastrointestinal disease selected from inflammatory bowel disease (IBD), Crohn's disease, and celiac disease.

19. The method of claim 10, comprising further administering one or more anti-inflammatory agent, molecule or cytokine to a subject or animal.

20. (canceled)

21. The method of claim 192, wherein the one or more anti-inflammatory agent, molecule or cytokine is selected from one or more anti-inflammatory, nonsteroidal anti-inflammatory drug (NSAID), steroid, biologic, antibiotic, or anti-diarrheal agent.

22. (canceled)

23. The method of claim 10, further comprising administering an effective amount of a prebiotic, wherein the prebiotic is selected from mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

24. (canceled)

25. The method of claim 10, wherein the composition or strains comprises a ratio of isolated first Lactobacillus reuteri strain to isolated second Lactobacillus reuteri strain of 0.75-1.5:1, or wherein the composition or strains comprises about equal amounts of the isolated first Lactobacillus reuteri strain and the isolated second Lactobacillus reuteri strain.

26. (canceled)

27. The method of claim 10, wherein the composition or strains are formulated as animal feed, feed additive, food ingredient, water additive, water-mixed additive, consumable solution, consumable spray additive, consumable solid, consumable gel, injection, or combinations thereof.

28. (canceled)

29. The method of claim 10, wherein the composition comprises the isolated first Lactobacillus reuteri strain in an amount of about 10²-10⁸ CFU/kg of the composition, about 10⁴-10⁷ CFU/kg of the composition, or about 10³-10⁵ CFU/kg of the composition, and/or comprises the isolated second Lactobacillus reuteri strain in an amount of about 10²-10⁸ CFU/kg of the composition, about 10⁴-10⁷ CFU/kg of the composition, or about 10³-10⁵ CFU/kg of the composition.

30. (canceled)

31. The method of claim 10, wherein the composition comprises the isolated first Lactobacillus reuteri strain in an amount of about 10⁷ CFU/kg of the composition, and/or the isolated second Lactobacillus reuteri strain in an amount of about 10⁷ CFU/kg of the composition.

32. (canceled)

33. The composition method of claim 10, wherein the animal is a bird, a human, or a non-human mammal.

34. The method of claim 33, wherein the animal is a farm animal or food production animal, a domestic animal, or wherein the bird is poultry.

35. (canceled)

36. (canceled)

37. The method of claim 10, wherein the composition or strains are administered orally or by injection, optionally by food administration or as a food or feed additive, or by direct ingestion in a powder, liquid, emulsion or pill.

38. (canceled)

39. (canceled)

40. The method of claim 10, wherein the composition or strains are administered by in ovo administration, or by spray administration.

41. (canceled)

42. (canceled)

43. An isolated biosynthetic gene cluster (BGC), particularly a polyketide synthase (PKS) BGC, capable of producing an AhR-activating metabolite.

44. The biosynthetic gene cluster (BGC) of claim 43, wherein the PKS BGC comprises the nucleic acid set out in SEQ ID NO: 77, wherein the PKS BGC comprises nucleic acid encoding one or more polypeptide selected from SEQ ID NOs: 78-85 and 87-92, or wherein the PKS BGC comprises nucleic acid encoding the polypeptides SEQ ID NOs: 78-85 and 87-92.

45. (canceled)

46. (canceled)

47. The biosynthetic gene cluster (BGC) of claim 43 comprising an Ahr-metabolite synthesizing cluster of genes encoding proteins SEQ ID NO: 78-85 and SEQ ID NO: 86-92.

48. An isolated plasmid comprising nucleic acid encoding one or more polypeptide selected from SEQ ID NOs: 78-92.

49. (canceled)

50. The plasmid of claim 48, wherein the encoded polypeptides are capable of synthesizing an Ahr-metabolite.

51. The biosynthetic gene cluster (BGC) of claim 47, wherein the Ahr-metabolite is capable of increasing IL-22 levels in the intestine in an animal, wherein the Ahr-metabolite is capable of ameliorating or reducing intestinal inflammation, or wherein the Ahr-metabolite is capable of reducing or alleviating increased intestinal permeability.

52. (canceled)

53. (canceled)

54. A method for expressing or producing an Ahr-metabolite via controlled expression or inducible expression of the PKS gene cluster of claim 43.

55. (canceled)
